(19) United States
(12) Patent Application Publication
(10) Pub. No.: US 2008/0311126 A1
(43) Pub. Date: Dec. 18, 2008

(54) Antisense Oligonucleotides Directed to Ribonucleotide Reducatase R2 and Uses Thereof in Combination Therapies for the Treatment of Cancer

(75) Inventors: Aiping H. Young, Toronto (CA); Jim A. Wright, Oakville (CA); Yoon Lee, Mississauga (CA)

Correspondence Address: CLARK & ELBING LLP, 101 FEDERAL STREET, BOSTON, MA, 02110, US

(73) Assignee: Genesense Technologies, Inc, Toronto (CA)

(21) Appl. No.: 10/585,772

(22) PCT Filed: Jan. 12, 2005
(86) PCT No.: PCT/CA2005/000040

§ 371 Date: Nov. 13, 2007

Related U.S. Application Data
(60) Provisional application No. 60/535,496, filed on Jan. 12, 2004.
(60) Provisional application No. 60/602,817, filed on Aug. 18, 2004.

(51) Int. Cl. A61K 39/395; A61K 31/7088; A61K 39/00; A61P 31/00

(52) U.S. Cl. 424/155.1; 514/44; 424/277.1

(57) ABSTRACT

Combination products comprising an antisense oligonucleotide against the gene encoding a mammalian ribonucleotide reductase R2 protein and one or more immunotherapeutic agents, such as cytokines, non-cytokine adjuvants, monoclonal antibodies and cancer vaccines. The combinations can further comprise one or more chemotherapeutic agents. Methods of treating cancer in a mammal using the combinations are also provided.

### FIELD OF THE INVENTION

The present invention pertains to the field of cancer therapeutics and in particular to combinations of an antisense oligonucleotide and one or more immunotherapeutic agents for the treatment of cancer.

### BACKGROUND OF TEE INVENTION

The first unique step leading to DNA synthesis is the conversion of ribonucleotides to their corresponding deoxyribonucleotides, a reaction that is catalyzed in a cell cycle specific manner by the housekeeping gene ribonucleotide reductase [Lewis et al. J. Cell Physiol. 94:287-2981978; Reichard, Science 60:1773-1777, 1993; Wright, Encyl. Pharmacol. Therapeut. 128:89-111, 19.89; Wright et al., Biochem. Cell Biol. 68:1364-1371 1990; Stubbe, Ann. Rev. Biochem. 58:257-285, 1989]. The mammalian enzyme is composed of two dissimilar dimeric protein subunits often called R1 and R2, both of which are required for enzymatic activity, and which are encoded by two different genes located on different chromosomes [Bjorklund et al., Proc. Natl. Acad. Sci. USA 90:11322-11326, 1993; Tonin et al., Cytogenet Cell Genet. 45:102-108, 1987].

The expression of ribonucleotide reductase, and in particular the R2 subunit, is elevated in transformed cells exposed to tumour promoters, or to transforming growth factors in growth factor mediated mechanisms of tumour progression [Amara et al., J. Biol. Chem. 271:20126-20131, 1996; Chen et al., EMBO J. 12:3977-3986, 1993; Amara et al., Nucleic Acids Res. 23:1461-1467, 1995]. These studies are in tumour cells obtained from rodent and human tissues [Weber, Cancer Res. 43:3466-3492, 1983; Wright et al., Encyl. Pharmacol. Therapeut. 128:89-111, 1989; Saeki, et al., Int. J. Oncol. 6:523-529, 1995; Jenson et al., Proc. Nat. Acad. Sci. USA 91:9257-9261, 1994], and in cultured cells selected for resistance to anti-tumour agents such as hydroxyurea [Lewis et al., J Cell Physiol. 97:87-97, 1978; Wright et al., Drug Resistance in Mammalian Cells, Boca Raton, Fla.; CRC Press, Inc; 15-27, 1989]. These findings suggest that interference with the expression of ribonucleotide reductase may be a useful approach to inhibit the proliferation of tumour cells.

In the last few years, advances in nucleic acid chemistry and gene transfer have inspired new approaches to engineer specific interference of gene expression. Antisense technology has been the most commonly described approach in protocols designed to achieve gene-specific interference and many antisense compounds have now entered clinical trials [see review in Holmlund, Ann N Y Acad Sci. 1002:244-51, 2003].

Antisense oligonucleotides specifically targeted against ribonucleotide reductase have been described, for example in, International Patent Application Nos. PCT/CA97/00454 and PCT/CA00/00120.

Immunotherapy is a fairly new approach to cancer therapy and involves directly or indirectly stimulating or enhancing the immune system's responses to cancer cells. Immunotherapy is also referred to as immunologic therapy, biological therapy, biological response modifier therapy and biotherapy and includes such diverse strategies as therapeutic vaccines (so-called “active immunotherapy”) and administration of biological agents such as cytokines and monoclonal antibodies (“passive immunotherapy”).

A number of immunotherapeutic approaches have obtained regulatory approval and are currently in use in clinical settings including the monoclonal antibodies trastuzumab (Herceptin), which was approved for the treatment of metastatic breast cancer, and rituximab (Rituxan), for the treatment of follicular B-cell lymphomas, as well as the non-cytokine adjuvant Levamisole, used in the treatment of colorectal cancer, and the cytokines interferon alpha, approved for use against chronic myelogenous leukemia (CML), multiple myeloma, non-Hodgkin's lymphoma, malignant melanoma, AIDS-Related Kaposi's sarcoma, hairy cell leukemia and basal cell carcinoma, and interleukin-2 (IL-2), approved for use in the treatment of metastatic renal cell carcinoma.

A study conducted by the Medical Research Council compared the efficacy of medroxyprogesterone acetate (MPA) and interferon in renal cell carcinoma. The study showed that interferon therapy gave an improvement in 1-year survival of 12% (MPA 31% survival, interferon 43% survival). Most patients tolerated the drug's side effects with continued therapy (Medical Research Council Renal Cancer Collaborators, Lancet (1999) 353(9146):14-17).

Combination of a cytokine with a standard chemotherapeutic may also provide benefits. A recent study has shown that IL-2 in combination with 13-cis retinoic acid prolonged the disease-free and overall survival in patients with recurrent ovarian cancer (Recchia, et al., Proc. 2004 European Society of Medical Oncology Congress, Vienna, Abst. # 491P). Other studies involving combinations of a cytokine with a chemotherapeutic have been conducted in renal cell carcinoma (see review by Bleumer et al. Eur Urol. 2003 44(1):65-75). Interferon alpha in combination combination with vinblastine was shown to be superior to vinblastine alone, providing a median survival of 67.6 weeks for the combination treated patients and 37.8 weeks for the patients receiving vinblastine alone (Pyrhonen et al., J Clin Oncol. 1999 17(9):2859-67). The combinations of interleukin plus interferon alpha (Negrier et al., Ann Oncol. 2002 13(9):1460-8; Tourani et al., J Clin Oncol. 2003 21(21):3987-94), interferon plus CCI-779 (Dutcher et al., Proc Am Soc Clin Oncol 2003. 22: 213 (Abstr 854)), interferon plus all-trans retinoic acid (Goldberg et al., Cancer 2002 95(6):1220-7), interferon alpha plus levamisole (Aksoy, Int Urol Nephrol. 2001 33(3):457-9), and interleukin plus interferon alpha in combination with 5-FU (Atzpodien et al., Cancer. 2002 95(5):1045-50; Van Herpen et al Br J Cancer. 2000 February; 82(4):772-6; Negrier et al., J Clin Oncol. 2000 18(24):4009-15) have also demonstrated efficacy against renal cancer. Recently Motzer et al. published a retrospective analysis on four hundred sixty-three previously untreated patients with advanced renal cell carcinoma who were treated with interferon alone or as part of combination therapy. Analysis showed that the 4-month and 6-month progression-free survival rates associated with interferon therapy were 55% and 42%, respectively (Motzer, J Clin Oncol. 2002 20(1):289-96).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide antisense oligonucleotides directed to ribonucleotide reductase R2 and uses thereof in combination therapies for the treatment of cancer. In accordance with one aspect of the present invention, there is provided a combination product for use in the treatment of cancer in a mammal, said combination product comprising: an antisense oligonucleotide of between 7 and 100 nucleotides in length comprising at least 7 consecutive nucleotides complementary to a mammalian ribonucleotide reductase R2 subunit mRNA and one or more immunotherapeutic agents.

In accordance with another aspect of the present invention, there is provided a method of treating cancer in a mammal comprising administering to said mammal a combination product comprising (a) an antisense oligonucleotide of between 7 and 100 nucleotides in length comprising at least 7 consecutive nucleotides complementary to a mammalian ribonucleotide reductase R2 subunit mRNA, and (b) one or more immunotherapeutic agents.

In accordance with another aspect of the present invention, there is provided a use of an antisense oligonucleotide of between 7 and 100 nucleotides in length comprising at least 7 consecutive nucleotides complementary to a mammalian ribonucleotide reductase R2 subunit mRNA and one or more immunotherapeutic agents in the manufacture of a medicament for the treatment of cancer in a mammal.

In accordance with another aspect of the present invention, there is provided a pharmaceutical kit comprising a combination product for the treatment of cancer, said combination product comprising (a) an antisense oligonucleotide of between 7 and 100 nucleotides in length comprising at least 7 consecutive nucleotides complementary to a mammalian ribonucleotide reductase R2 subunit mRNA, and (b) one or more immunotherapeutic agents.

In accordance with another aspect of the present invention, there is provided a combination product for use in the treatment of renal cancer in a subject, said combination product comprising: an antisense oligonucleotide of between 7 and 100 nucleotides in length comprising at least 7 consecutive nucleotides complementary to SEQ ID NO:1 and one or more cytokines.

### BRIEF DESCRIPTION OF THE FIGURES

FIG. 1 depicts the anti-proliferative effects of interferon alpha in vitro in human renal carcinoma cell lines (Caki-1 and A498);

FIG. 2 depicts the effects of SEQ ID NO:1 alone and in combination with interferon alpha on Caki-1 renal tumour growth in SCID mice;

FIG. 3 depicts the effects of SEQ ID NO:1 alone and in combination with interferon alpha on Caki-1 renal tumour growth in SCID mice;

FIG. 4 depicts the effects of SEQ ID NO:1 alone and in combination with interferon alpha on Caki-1 renal tumour growth in SCID mice;

FIG. 5 depicts the effects of SEQ ID NO:1 alone and in combination with interferon alpha on Cali-1 renal carcinoma growth in SCID mice;

FIG. 6 depicts the effects of SEQ ID NO:1 alone and in combination with interferon alpha on A498 renal carcinoma growth in SCID mice;

FIG. 7 depicts the effects of SEQ ID NO:1 alone and in combination with interferon alpha on A498 renal tumour growth in SCID mice; and

FIG. 8 depicts the effects of SEQ ID NO:1 alone and in combination with interleukin-2 on Caki-1 renal tumour growth in SCID mice.

FIG. 9 depicts effects of SEQ ID NO:1 in combination with a chemotherapeutic on HT-29 colon tumour growth in nude mice;

FIG. 10 depicts effects of SEQ ID NO:1 in combination with a chemotherapeutic on HT-29 colon tumour growth in nude mice;

FIG. 11 depicts effects of SEQ ID NO:1 in combination with a chemotherapeutic on HT-29 colon tumour growth in nude mice;

FIG. 12 depicts effects of SEQ ID NO:1 in combination with a chemotherapeutic on HT-29 colon tumour growth in nude mice;

FIG. 13 depicts effects of SEQ ID NO:1 in combination with a chemotherapeutic on Caki-1 renal tumour growth in SCID mice;

FIG. 14 depicts effects of SEQ ID NO:1 in combination with a chemotherapeutic on prostatic tumour growth in SCID mice;

FIG. 15 depicts effects of SEQ ID NO:1 in combination with a chemotherapeutic on prostatic tumour growth in SCID mice;

FIG. 16 depicts effects of SEQ ID NO:1 in combination with a chemotherapeutic on A2058 melanoma growth in CD-1 nude mice;

FIG. 17 depicts effects of SEQ ID NO:1 in combination with a chemotherapeutic on breast tumour growth in CD-1 nude mice;

FIG. 18 depicts effects of SEQ ID NO:1 in combination with a chemotherapeutic on ovary tumour growth in CD-1 nude mice;

FIG. 19 depicts effects of SEQ ID NO:1 in the treatment of human pancreatic carcinoma in CD-1 nude mice;

FIG. 20 depicts effects of SEQ ID NO:1 in the treatment of human cervix epitheloid carcinoma resistant to hydroxyurea (HU) in SCID mice;

FIG. 21 depicts effects of SEQ ID NO:1 in the treatment of human breast adenocarcinoma resistant to cisplatin in SCID mice;

FIG. 22 depicts effects of SEQ ID NO:1 in the treatment of human breast adenocarcinoma resistant to cisplatin in SCID mice;

FIG. 23 depicts effects of SEQ ID NO:1 in the treatment of human breast adenocarcinoma resistant to taxol in SCID mice;

FIG. 24 depicts effects of SEQ ID NO:1 in the treatment of human breast adenocarcinoma resistant to taxol in SCID mice;

FIG. 25 depicts effects of SEQ ID NO:1 in the treatment of human promyelocytic leukaemia resistant to taxol in SCID mice;

FIG. 26 depicts effects of SEQ ID NO:1 in the treatment of LS513, human multi-drug resistant colon adenocarcinoma in SCID mice; and

FIG. 27 depicts the sequence of the human ribonucleotide reductase R2 mRNA [SEQ ID NO:105].

### DETAILED DESCRIPTION OF THE INVENTION

The present invention provides for combination products comprising one or more antisense oligonucleotides against the gene encoding the R2 subunit of a mammalian ribonucleotide reductase protein and one or more immunotherapeutic agents for the treatment of cancer. Combination therapy with an antisense oligonucleotides targeted to the ribonucleotide reductase R2 gene and an immunotherapeutic agent have been found to be more effective in decreasing the growth of neoplastic cells than either the antisense oligonucleotide or the immunotherapeutic agent(s) alone. The combination products of the present invention can further comprise one or more chemotherapeutic agents.

In the context of the present invention, a “combination product” or “combination” comprises an antisense oligonucleotide targeted against a mammalian ribonucleotide reductase R2 gene and one or more immunotherapeutic agents. The antisense oligonucleotide and the immunotherapeutic agent(s) can be administered separately, sequentially, simultaneously or in a mixture to the subject undergoing treatment. Thus, a combination product can contain, for example, multiple, separate dosage units, with each active ingredient of the combination being provided in an individual dosage unit, or multiple dosage units, with each unit comprising one or more active ingredients, or single dosage units, which contain a fixed ratio of all the active ingredients of the combination.

The present invention further provides for the use of combinations comprising an antisense oligonucleotide targeted to the ribonucleotide reductase R2 gene and one or more immunotherapeutic agents in combination therapies for the treatment of various cancers. The invention further provides for methods of treating cancer in a mammal comprising administering an effective amount of a combination an antisense oligonucleotide targeted to the ribonucleotide reductase R2 gene and one or more immunotherapeutic agent. The combination therapy can be a first-line treatment, or it can be a part of an adjuvant therapy for a cancer patient who has already undergone a primary therapy.

### DEFINITIONS

Unless defined otherwise, all technical and scientific terms used herein have the same meaning as commonly understood by one of ordinary skill in the art to which this invention pertains.

The term “antisense oligonucleotide,” as used herein, refers to an oligonucleotide comprising a sequence that is complementary to the mRNA transcribed from a target gene. In the context of the present invention, the target gene is the gene encoding a mammalian ribonucleotide reductase R2 protein.

The term “oligonucleotide,” as used herein, means a polymeric form of nucleotides of at least 7 nucleotides in length comprising either ribonucleotides or deoxynucleotides or modified forms of either type of nucleotide. The term includes single and double stranded forms of DNA or RNA.

The term “immunotherapeutic agent,” as used herein, refers to a compound, composition or treatment that indirectly or directly enhances, stimulates or augments the body's immune response against cancer cells and/or that lessens the side effects of other anticancer therapies. Examples of common immunotherapeutic agents known in the art include, but are not limited to, cytokines, cancer vaccines, monoclonal antibodies and non-cytokine adjuvants.

The term “selectively hybridise” as used herein refers to the ability of a nucleic acid molecule to bind detectably and specifically to a second nucleic acid molecule. Oligonucleotides selectively hybridise to target nucleic acid strands under hybridisation and wash conditions that minimise appreciable amounts of detectable binding to non-specific nucleic acid molecules. High stingency conditions can be used to achieve selective hybridisation conditions as known in the art and discussed herein.

Typically, hybridisation and washing conditions are performed at high stringency according to conventional hybridisation procedures. Washing conditions are typically 1-3×SSC, 0.1-1% SDS, 50-70° C. with a change of wash solution after about 5-30 minutes.

The term “corresponds to” as used herein with reference to nucleic acid sequences means a polynucleotide sequence that is identical to all or a portion of a reference polynucleotide sequence. In contradistinction, the term “complementary to” is used herein to mean that the polynucleotide sequence is identical to all or a portion of the complement of a reference polynucleotide sequence. For illustration, the nucleotide sequence “TATAC” corresponds to a reference sequence “TATAC” and is complementary to a reference sequence “GTATA”.

The following terms are used herein to describe the sequence relationships between two or more polynucleotides: “reference sequence,” “window of comparison,” “sequence identity,” “percent (%) sequence identity” and “substantial identity.” A “reference sequence” is a defined sequence used as a basis for a sequence comparison; a reference sequence may be a subset of a larger sequence, for example, as a segment of a full-length cDNA, mRNA or gene sequence, or may comprise a complete cDNA, mRNA or gene sequence. Generally, a reference polynucleotide sequence is at least 20 nucleotides in length, and often at least 50 nucleotides in length.

A “window of comparison”, as used herein, refers to a conceptual segment of the reference sequence of at least 15 contiguous nucleotide positions over which a candidate sequence may be compared to the reference sequence and wherein the portion of the candidate sequence in the window of comparison may comprise additions or deletions (i.e. gaps) of 20 percent or less as compared to the reference sequence (which does not comprise additions or deletions) for optimal alignment of the two sequences. The present invention contemplates various lengths for the window of comparison, up to and including the full length of either the reference or candidate sequence. In one embodiment, the window of comparison is the full length of the candidate sequence. Optimal alignment of sequences for aligning a comparison window may be conducted using the local homology algorithm of Smith and Waterman (Adv. Appl. Math. (1981) 2:482), the homology alignment algorithm of Needleman and Wunsch (J. Mol. Biol. (1970) 48:443), the search for similarity method of Pearson and Lipman (Proc. Natl. Acad. Sci. (U.S.A) (1988) 85:2444), using computerised implementations of these algorithms (such as GAP, BESTFIT, FASTA, and TFASTA in the Wisconsin Genetics Software Package Release 7.0, Genetics Computer Group, 573 Science Dr., Madison, Wis.), using publicly available computer software such as ALIGN or Megalign (DNASTAR), or by inspection. The best alignment (i.e. resulting in the highest percentage of identity over the comparison window) is then selected.

The term “sequence identity” means that two polynucleotide sequences are identical (i.e. on a nucleotide-by-nucleotide basis) over the window of comparison.

The term “percent (%) sequence identity,” as used herein with respect to a reference sequence is defined as the percentage of nucleotide residues in a candidate sequence that are identical with the residues in the reference polynucleotide sequence over the window of comparison after optimal alignment of the sequences and introducing gaps, if necessary, to achieve the maximum percent sequence identity, without considering any conservative substitutions as part of the sequence identity.

The term “substantial identity” as used herein denotes a characteristic of a polynucleotide sequence, wherein the polynucleotide comprises a sequence that has at least 5.0% sequence identity as compared to a reference sequence over the window of comparison. In various embodiments of the invention, polynucleotide sequences having at least 60% sequence identity, at least 70% sequence identity, at least 80% sequence identity, and at least 90% sequence identity as compared to a reference sequence over the window of comparison are considered to have substantial identity with the reference sequence.

The terms “therapy” and “treatment,” as used interchangeably herein, refer to an intervention performed with the intention of improving a recipient's status. The improvement can be subjective or objective and is related to the amelioration of the symptoms associated with, preventing the development of, or altering the pathology of a disease, disorder or condition being treated. Thus, the terms therapy and treatment are used in the broadest sense, and include the prevention (prophylaxis), moderation, reduction, and curing of a disease, disorder or condition at various stages. Prevention of deterioration of a recipient's status is also encompassed by the term. Those in need of therapy/treatment include those already having the disease, disorder or condition as well as those prone to, or at risk of developing, the disease, disorder or condition and those in whom the disease, disorder or condition is to be prevented.

The term “ameliorate” or “amelioration” includes the arrest, prevention, decrease, or improvement in one or more the symptoms, signs, and features of the disease being treated, both temporary and longterm.

The term “subject” or “patient” as used herein refers to a mammal in need of treatment.

Administration of the compounds of the invention “in combination with.” one or more further therapeutic agents, is intended to include simultaneous (concurrent) administration and consecutive administration. Consecutive administration is intended to encompass administration of the therapeutic agent(s) and the compound(s) of the invention to the subject in various orders and via various routes.

As used herein, the term “about” refers to a +/−10% variation from the nominal value. It is to be understood that such a variation is always included in any given value provided herein, whether or not it is specifically referred to.

### Antisense Oligonucleotides

### Selection and Characteristics

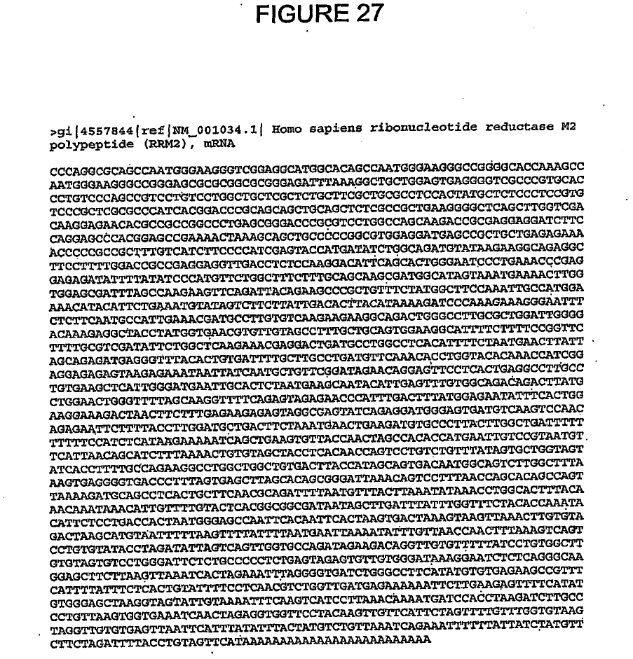

The antisense oligonucleotides of the present invention are targeted to a gene encoding the R2 subunit of a mammalian ribonucleotide reductase protein. The antisense oligonucleotides are thus complementary to a portion of a mRNA transcribed from a mammalian ribonucleotide reductase R2 subunit gene. The sequences of various mammalian ribonucleotide reductase mRNAs are known in the art. For example, the mRNA sequences for the human ribonucleotide reductase R2 subunit (GenBank Accession No. NM_{—}001034) is available from the GenBank database maintained by the NCBI and are provided herein as SEQ ID NO:105 (FIG. 27). The sequences of other mammalian ribonucleotide reductase mRNAs are also available from this database, for example, NM_{—}009104 Mouse (Mus musculus) R2 subunit and X68127 Golden hamster (Mesocricetus auratus) R2 subunit.

In one embodiment of the present invention, the antisense oligonucleotides are targeted to a human ribonucleotide reductase R2 subunit gene. In another embodiment, the antisense oligonucleotides comprise a sequence that is complementary to a portion of a human ribonucleotide reductase R2 subunit mRNA. In a further embodiment, the antisense oligonucleotides comprise a sequence that is complementary to a portion of the sequence as set forth in SEQ ID NO:105.

The antisense oligonucleotides of the present invention comprise a sequence of at least 7 contiguous nucleotides that are complementary to a portion of the selected mammalian ribonucleotide reductase R2 mRNA. In one embodiment, the antisense oligonucleotides comprise a sequence of at least 7 contiguous nucleotides that are complementary to a portion of the human ribonucleotide reductase R2 mRNA.

Examples of suitable antisense oligonucleotides for inclusion in the combinations of the present invention include those disclosed in U.S. Pat. Nos. 5,998,383 and 6,121,000 (herein incorporated by reference) which are targeted to the ribonucleotide reductase R2 gene. Exemplary sequences are provided in Table 1. In one embodiment of the present invention, the antisense-oligonucleotide comprises at least 7 consecutive nucleotides of any one of the antisense oligonucleotide sequences set forth in Table 1. In another embodiment, the antisense oligonucleotides comprise a sequence of at least 7 contiguous nucleotides that are complementary to portion of the coding region of a mammalian ribonucleotide reductase R2 gene or mRNA. In a further embodiment, the antisense oligonucleotide comprises at least 7 consecutive nucleotides of the antisense oligonucleotide represented by the sequence:

| | |
|---|---|
| 5′-GGCTAAATCGCTCCACCAAG-3′ | [SEQ ID NO: 1] |

TABLE 1

Exemplary Antisense Oligonucleotides Targeted
to the Human Ribonucleotide Reductase R2 mRNA

| | | | | |
|---|---|---|---|---|
| SEQ | | | | dG |
| ID | | | Tm | kDa/ |
| NO | Name | Sequence 5′ - 3′ | ° C. | mol |

| | | | | |
|---|---|---|---|---|
| 1 | AS-II-626-20 | GGCTAAATCGCTCCACCAAG | 53.9 | −40.3 |
| 4 | AS-II-6-20 | ACCCTTCCCATTGGCTGCGC | 62.8 | −45.5 |
| 5 | AS-II-13-20 | G*CC*TCCG*ACC*CTTC*CC*ATT*G | 60.1 | −43.7 |
| 6 | AS-II-14-20 | TGCCTCCGACCCTTCCCATT | 60.1 | −43.7 |
| 7 | AS-II-16-18 | TGCCTCCGACCCTTCCCA | 58.4 | −40.3 |
| 8 | AS-II-75-20 | C*GCG*CGC*TCC*CGG*CCC*TTC*C | 72.7 | −53.7 |
| | (partial) | | | |
| 9 | AS-II-75-20 | CGCGCGCTCCCGGCCCTTCC | 72.7 | −53.7 |
| 10 | AS-II-79-14 | CGCGCTCCCGGCCC | 59.1 | −38.8 |
| 11 | AS-II-109-20 | C*CCC*TCAC*TCC*AGC*AGC*CT*T | 57.9 | −41.8 |
| 12 | AS-II-110-20 | ACCCCTCACTCCAGCAGCCT | 57.3 | −41.2 |
| 13 | AS-II-114-20 | GGCGACCCCTCACTCCAGCA | 61.8 | −43.2 |
| 14 | AS-II-127-12 | GCACGGGCGACC | 41.7 | −28.8 |
| 15 | AS-II-130-20 | TGGGACAGGGTGCACGGGCG | 67.6 | −46.7 |
| 16 | AS-II-134-20 | GACGGCTGGGACAGGGTGCA | 62.6 | −43.2 |
| 17 | AS-II-151-20 | GAGCAGCCAGGACAGGACGG | 59.3 | −41.7 |
| 18 | AS-II-163-20 | G*CG*AAG*CAG*AGC*GAG*CAGC*C | 62.1 | −44.3 |
| 19 | AS-II-166-20 | GCAGCGAAGCAGAGCGAGCA | 61.4 | −43.1 |
| 20 | AS-II-185-20 | GGGAGAGCATAGTGGAGGCG | 56.0 | −40.9 |
| 21 | AS-II-189-20 | CGGAGGGAGAGCATAGTGGA | 54.1 | −39.4 |
| 22 | AS-II-201-20 | GCGAGCGGGACACGGAGGGA | 63.5 | −45.1 |
| 23 | AS-II-217-20 | CGGGTCCGTGATGGGCGCGA | 69.5 | −48.8 |
| 24 | AS-II-225-20 | AGCTGCTGCGGGTCCGTGAT | 61.4 | −43.6 |
| 25 | AS-II-253-14 | CCCCTTCAGCGGCG | 50.8 | −34.4 |
| 26 | AS-II-280-20 | CGGCGGCGTGTTCTCCTTGT | 61.8 | −44.2 |
| 27 | AS-II-288-12 | CGGCGGCGTGTT | 43.2 | −29.6 |
| 28 | AS-II-323-20 | TCCTCGCGGTCTTGCTGGCC | 64.1 | −45.5 |
| 29 | AS-II-344-20 | CCGTGGGCTCCTGGAAGATC | 58.0 | −41.9 |
| 30 | AS-II-362-20 | CTGCTTTAGTTTTCGGCTCC | 51.2 | −39.2 |
| 31 | AS-II-391-17 | CGGCTCATCCTCCACGC | 54.5 | −37.3 |
| 32 | AS-II-404-20 | GGTTTTCTCTCAGCAGCGGC | 56.4 | −41.4 |
| 33 | AS-II-412-20 | GCGGCGGGGGTTTTCTCTCA | 62.8 | −45.8 |
| 34 | AS-II-414-20 | AAGCGGCGGGGGTTTTCTCT | 60.7 | −45.8 |
| 35 | AS-II-425-20 | GGAAGATGACAAAGCGGCGG | 59.1 | −43.0 |
| 36 | AS-II-439-20 | ATGGTACTCGATGGGGAAGA | 50.8 | −37.8 |
| 37 | AS-II-472-20 | AGCCTCTGCCTTCTTATACA | 46.1 | −35.8 |
| 38 | AS-II-494-20 | CCTCCTCGGCGGTCCAAAAG | 60.4 | −44.3 |
| 39 | AS-II-496-16 | TCCTCGGCGGTCCAAA | 54.8 | −37.0 |
| 40 | AS-II-549-20 | TATCTCTCCTCGGGTTTCAG | 48.4 | −36.7 |
| 41 | AS-II-579-20 | GCAAAGAAAGCCAGAACATG | 50.0 | −37.2 |
| 42 | AS-II-619-20 | TCGCTCCACCAAGTTTTCAT | 52.1 | −38.3 |
| 43 | AS-II-634-20 | AACTTCTTGGCTAAATCGCT | 48.0 | −37.6 |
| 44 | AS-II-667-20 | GAAGCCATAGAAACAGCGGG | 53.9 | −40.3 |
| 45 | AS-II-784-20 | GACACAAGGCATCGTTTCAA | 50.9 | −36.8 |
| 46 | AS-II-798-20 | TCTGCCTTCTTCTTGACACA | 48.0 | −34.9 |
| 47 | AS-II-816-20 | ATCCAGCGCAAGGCCCAGTC | 60.9 | −43.7 |
| 48 | AS-II-861-20 | GCAAAGGCTACAACACGTTC | 50.0 | −37.1 |
| 49 | AS-II-890-20 | AACCGGAAAAGAAAATGCCT | 52.2 | −40.4 |
| 50 | AS-II-909-20 | CAGAATATCGACGCAAAAGA | 48.2 | −36.5 |
| 51 | AS-II-933-20 | GGCATCAGTCCTCGTTTCTT | 50.8 | −37.7 |
| 52 | AS-II-981-20 | TGTAAACCCTCATCTCTGCT | 46.2 | −35.0 |
| 53 | AS-II-1001-20 | TCAGGCAAGCAAAATCACAG | 51.3 | −37.2 |
| 54 | AS-II-1006-20 | GAACATCAGGCAAGCAAAAT | 49.4 | −37.1 |
| 55 | AS-II-1023-20 | TTGTGTACCAGGTGTTTGAA | 45.9 | −33.9 |
| 56 | AS-II-1040-20 | GTCTCTCCTCCGATGGTTTG | 51.1 | −37.7 |
| 57 | AS-II-1048-20 | TTCTCTTACTCTCTCCTCCG | 45.2 | −35.0 |
| 58 | AS-II-1144-20 | GTATTGCTTCATTAGAGTGC | 41.6 | −33.0 |
| 59 | AS-II-1182-20 | CCCAGTTCCAGCATAAGTCT | 48.4 | −36.5 |
| 60 | AS-II-1197-20 | AAAACCTTGCTAAAACCCAG | 48.3 | −37.8 |
| 61 | AS-II-1217-20 | CAAATGGGTTCTCTACTCTG | 43.7 | −33.8 |
| 62 | AS-II-1224-20 | ATAAAGTCAAATGGGTTCTC | 42.6 | −34.0 |
| 63 | AS-II-1254-20 | TTAGTCTTTCCTTCCAGTGA | 43.8 | −33.9 |
| 64 | AS-II-1278-20 | TCGCCTACTCTCTTCTCAAA | 46.8 | −35.6 |
| 65 | AS-II-1288-20 | CCTCTGATACTCGCCTACTC | 45.6 | −35.1 |
| 66 | AS-II-1302-20 | GACATCACTCCCATCCTCTG | 48.7 | −35.3 |
| 67 | AS-II-1335-20 | GCATCCAAGGTAAAAGAATT | 45.6 | −36.1 |
| 68 | AS-II-1338-20 | TCAGCATCCAAGGTAAAAGA | 47.4 | −35.9 |
| 69 | AS-II-1342-20 | GAAGTCAGCATCCAAGGTAA | 46.7 | −35.3 |
| 70 | AS-II-1345-20 | TTAGAAGTCAGCATCCAAGG | 47.0 | −35.6 |
| 71 | AS-II-1362-20 | GCACATCTTCAGTTCATTTA | 42.4 | −32.8 |
| 72 | AS-II-1364-20 | GGGCACATCTTCAGTTCATT | 48.9 | −36.2 |
| 73 | AS-II-1381-20 | AAAAATCAGCCAAGTAAGGG | 48.1 | −38.0 |
| 74 | AS-II-1390-20 | ATGGAAAAAAAAAATCAGCC | 48.1 | −38.0 |
| 75 | AS-II-1438-20 | TTCATGGTGTGGCTAGTTGG | 50.8 | −36.8 |
| 76 | AS-II-1499-20 | AGGACTGGTTGTGAGGTAGC | 48.1 | −35.7 |
| 77 | AS-II-1517-20 | CCAGCACTATAAACAGACAG | 42.2 | −32.8 |
| 78 | AS-II-1538-20 | TTCTGGCAAAAGGTGATACT | 46.5 | −35.6 |
| 79 | AS-II-1560-20 | GTAAGTCACAGCCAGCCAGG | 52.2 | −37.8 |
| 80 | AS-II-1581-20 | ACTGCCATTGTCACTGCTAT | 47.0 | −34.9 |
| 81 | AS-II-1659-20 | TGGCTGTGCTGGTTAAAGGA | 53.2 | −38.7 |
| 82 | AS-II-1666-20 | TTTTAACTGGCTGTGCTGGT | 50.0 | −37.2 |
| 83 | AS-II-1700-20 | ATTAAAATCTGCGTTGAAGC | 46.8 | −36.6 |
| 84 | AS-II-1768-20 | TATCGCCGCCGTGAGTACAA | 56.5 | −40.9 |
| 85 | AS-II-1773-20 | GCTATTATCGCCGCCGTGAG | 57.1 | −42.6 |
| 86 | AS-II-1775-12 | ATCGCCGCCGTG | 42.9 | −29.5 |
| 87 | AS-II-1790-20 | GAAACCAAATAAATCAAGCT | 43.4 | −34.9 |
| 88 | AS-II-1819-20 | TTAGTGGTCAGGAGAATGTA | 41.7 | −32.5 |
| 89 | AS-II-1976-20 | TGGCACCAACTGACTAATAT | 44.5 | −34.2 |
| 90 | AS-II-1989-20 | CCTGTCTTCTATCTGGCACC | 48.6 | −36.2 |
| 91 | AS-II-2009-20 | GCCACAGGATAAAAACACAA | 47.7 | −35.9 |
| 92 | AS-II-2026-20 | CCCAGGACACTACACAAGCC | 51.8 | −37.5 |
| 93 | AS-II-2044-20 | TCAGAGGGGGCAGAGAATCC | 55.4 | −40.2 |
| 94 | AS-II-2067-20 | TCCTTTATCCCACAACACTC | 46.3 | −35.0 |
| 95 | AS-II-2083-20 | CCTTGCCCTGAGAGATTCCT | 52.3 | −39.0 |
| 96 | AS-II-2083-20 | C*CT*TG*CC*CT*GA*GA*GA*TT*CC*T | 52.3 | −39.0 |
| | (Partial) | | | |
| 97 | AS-II-2128-20 | GGCCCAGATCACCCCTAAAT | 54.3 | −40.9 |
| 98 | AS-II-2151-20 | AAACGGCTTCTCACACATAT | 46.3 | −35.4 |
| 99 | AS-II-2164-20 | GAGAAATAAAATGAAACGGC | 46.2 | −36.6 |
| 100 | AS-II-2182-20 | CGTTGAGGAAAATACAGTGA | 45.1 | −34.3 |
| 101 | AS-II-2229A-20 | GCTCCCACATATGAAAACTC | 46.1 | −35.2 |
| 102 | AS-II-2372-20 | CACACAACCTACTTACACCA | 42.7 | −32.3 |
| 103 | AS-II-336-20 | TCCTGGAAGATCCTCCTCGC | — | — |
| 104 | AS-II-2229B-20 | TCCCACATATGAGAAAACTC | — | — |

Footnotes for Table 1:
Name includes the following: AS = antisense; II = R2; the first number indicates the first nucleotide position in the R2 mRNA sequence; the second number indicates the length of the sequence segment. Sequences were fully phosphorothioated unless partial phosphorothioation is indicated by “*”
Tm ° C. = melting temperature of oligonucleotide duplex formed.
dG = free energy values of oligonucleotide-complement dimer formation.

The antisense oligonucleotides in accordance with the present invention are selected such that the antisense sequence exhibits the least likelihood of forming duplexes, hairpins or dimers, and contains minimal or no homzoligomer/sequence repeats. The oligonucleotide may further contain a GC clamp. One skilled in the art will appreciate that these properties can be determined qualitatively using various computer modelling programs, for example, the program OLIGO® Primer Analysis Software, Version 5.0 (distributed by National Biosciences, Inc., Plymouth, Minn.).

In order to be effective, conventional antisense oligonucleotides are typically between 7 and 100 nucleotides in length. In one embodiment of the present invention, the antisense oligonucleotides are between about 7 and about 50 nucleotides in length. In another embodiment, the antisense oligonucleotides are between about 10 and about 50 nucleotides in length. In a further embodiment, the antisense oligonucleotides are between about 12 and about 50 nucleotides in length. In other embodiments, the antisense oligonucleotides are between about 7 and about 35 nucleotides in length, between about 10 and about 35 nucleotides, between about 12 and about 35 nucleotides and between about 12 and about 25 nucleotides in length.

It is understood in the art that an antisense oligonucleotide need not have 100% identity with the complement of its target sequence. The antisense oligonucleotides in accordance with the present invention have a sequence that is at least about 75% identical to the complement of their target sequence. In one embodiment of the present invention, the antisense oligonucleotides have a sequence that is at least about 90% identical to the complement of the target sequence. In another embodiment, they have a sequence that is at least about 95% identical to the complement of target sequence, allowing for gaps or mismatches of several bases. In a further embodiment, they are at least about 98% identical to the complement of the target sequence. Identity can be determined, for example, by using the BLASIN program of the University of Wisconsin Computer Group (GCG) software or provided on the NCBI website.

In the context of this invention, an oligonucleotide can be an oligomer or polymer of ribonucleic acid (RNA), deoxyribonucleic acid (DNA), or modified RNA or DNA, or combinations thereof. This term, therefore, includes oligonucleotides composed of naturally-occurring nucleobases, sugars and covalent internucleoside (backbone) linkages as well as oligonucleotides having non-naturally-occurring portions, which function similarly. Such modified oligonucleotides are often preferred over native forms because of desirable properties such at, for example, enhanced cellular uptake, enhanced affinity for nucleic acid target and increased stability in the presence of nucleases.

As is known in the art; a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Specific non-limiting examples of modified oligonucleotides useful in the present invention include oligonucleotides containing modified backbones or non-natural internucleoside linkages. As defined in this specification, oligonucleotides having modified backbones include both those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary antisense oligonucleotides having modified oligonucleotide backbones include, for example, those with one or more modified internucleotide linkages that are phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

In one embodiment of the present invention, the antisense oligonucleotide is a phosphorothioated oligonucleotide that comprises one or more phosphorothioate internucleotide linkages. In another embodiment, the antisense oligonucleotide comprises phosphorothioate internucleotide linkages that link the four, five or six 3′-terminal nucleotides of the oligonucleotide. In a further embodiment, the antisense oligonucleotide comprises phosphorothioate internucleotide linkages that link all the nucleotides of the oligonucleotide.

Exemplary modified oligonucleotide backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include morpholino linkages (formed in part from the sugar portion of a nucleoside); siloxane backbones; sulfide, sulfoxide and sulphone backbones; formacetyl and thioformacetyl backbones; methylene formacetyl and thioformacetyl backbones; alkene containing backbones; sulphamate backbones; methyleneimino and methylenehydrazino backbones; sulphonate and sulfonamide backbones; amide backbones; and others having mixed N, O, S and CH₂ component parts.

The present invention also contemplates modified oligonucleotides in which both the sugar and the internucleoside linkage of the nucleotide units are replaced with novel groups. The base units are maintained for hybridisation with an appropriate nucleic acid target compound. An example of such a modified oligonucleotide, which has been shown to have excellent hybridisation properties, is a peptide nucleic acid (PNA) [Nielsen et al., Science,. 254:1497-1500 (1991)]. In PNA compounds, the sugar-backbone of an oligonucleotide is replaced with an amide containing backbone, in particular an aminoethylglycine backbone. The nucleobases are retained and are bound directly or indirectly to aza-nitrogen atoms of the amide portion of the backbone.

The present invention also contemplates oligonucleotides comprising “locked nucleic acids” (LNAs), which are conformationally restricted oligonucleotide analogues containing a methylene bridge that connects the 2′-O of ribose with the 4′-C (see, Singh et al., Chem. Commun., 1998, 4:455-456). LNA and LNA analogues display very high duplex thermal stabilities with complementary DNA and RNA, stability towards 3′-exonuclease degradation, and good solubility properties. Synthesis of the LNA analogues of adenine, cytosine, guanine, 5-methylcytosine, thymine and uracil, their oligomerization, and nucleic acid recognition properties have been described (see Koshkin et al, Tetrahedron, 1998, 54:3607-3630). Studies of mismatched sequences show that LNA obey the Watson-Crick base pairing rules with generally improved selectivity compared to the corresponding unmodified reference strands.

Antisense oligonucleotides containing LNAs have been demonstrated to be efficacious and non-toxic (Wahlestedt et al., Proc. Natl. Acad. Sci. U.S.A., 2000, 97:5633-5638). In addition, the LNA/DNA copolymers were not degraded readily in blood serum and cell extracts.

LNAs form duplexes with complementary DNA or RNA or with complementary LNA, with high thermal affinities. The universality of LNA-mediated hybridization has been emphasized by the formation of exceedingly stable LNA:LNA duplexes (Kosbkin et al., J. Am. Chem. Soc., 1998, 120:13252-13253). LNA:LNA hybridization was shown to be the most thermally stable nucleic acid type duplex system, and the RNA-mimicking character of LNA was established at the duplex level. Introduction of three LNA monomers (T or A) resulted in significantly increased melting points toward DNA complements.

Synthesis of 2′-amino-LNA (Singh et al., J. Org. Chem., 1998, 63, 10035-10039) and 2′-methylamino-LNA has been described and thermal stability of their duplexes with complementary RNA and DNA strands reported. Preparation of phosphorothioate-LNA and 2′-thio-LNA have also been described (Kumar et al., Bioorg. Med. Chem. Lett., 1998, 8:2219-2222).

Modified oligonucleotides may also contain one or more substituted sugar moieties. For example, oligonucleotides may comprise sugars with one of the following substituents at the 2′ position: OH; F; O-, S-, or N-alkyl; O-, S-, or N-alkenyl; O- , S- or N-alkynyl; or O-alkyl-O-alkyl, wherein the allyl, alkenyl and alkynyl may be substituted or unsubstituted C₁ to C₁₀ alkyl or C₂ to C₁₀ alkenyl and alkynyl. Examples of such groups are: O[(CH₂)ₙO]ₘ CH₃, O(CH₂)ₙ OCH₃, O(CH₂)ₙNH₂, O(CH₂)ₙCH₃, O(CH₂)ₙONH₂, and O(CH₂)ₙ ON[(CH₂)ₙ CH₃)]₂, where n and m are from 1 to about 10. Alternatively, the oligonucleotides may comprise one of the following substituents at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂₁ NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Specific examples include 2′-methoxyethoxy (2′-O—CH₂ CH₂ OCH₃, also known as 2′-O-(2-methoxyethyl) or 2′-MOE) [Martin et al., Helv. Chim. Acta, 78:486-504 (1995)], 2′-dimethylaminooxyethoxy (O(CH₂)₂ ON(CH₃)₂ group, also known as 2′-DMAOE), 2′-methoxy (2′-O—CH₃), 2′-aminopropoxy (2′-OCH₂ CH₂ CH₂ NH₂) and 2′-fluoro (2′-F). In one embodiment of the present invention, the antisense oligonucleotide comprises at least one nucleotide comprising a substituted sugar moiety. In another embodiment, the antisense oligonucleotide comprises at least one 2′-O-(2-methoxyethyl) or 2′-MOE modified nucleotide.

Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include modifications to the nucleobase. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine-(5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. I., ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi, Y. S., (1993) Antisense Research and Applications, pp 276-278, Crooke, S. T. and Lebleu, B., ed., CRC Press, Boca Raton].

Another oligonucleotide modification included in the present invention is the chemical linkage to the oligonucleotide of one or more moieties or conjugates which enhance the activity, cellular distribution or cellular uptake of the oligonucleotide. Such moieties include, but are not limited to, lipid moieties such as a cholesterol moiety [Letsinger et al., Proc. Natl. Acad. Sci USA, 86:6553-6556 (1989)], cholic acid [Manoharan et al., Bioorg. Med. Chem. Let., 4:1053-1060 (1994)], a thioether, e.g. hexyl-5-tritylthiol [Manoharan et al., Ann. N.Y. Acad. Sci., 660:306-309 (1992); Manoharan et al., Bioorg. Med. Chem. Lett., 3:2765-2770 (1993)], a thiocholesterol [Oberhauser et al., Nucl. Acids Res., 20:533-538 (1992)], an aliphatic chain, e.g. dodecandiol or undecyl residues [Saisdn-Behmoaras et al., EMBO j., 10:1111-1118 (1991); Kabanov et al., FEBS Lett., 259:327-330 (1990); Svinarchuk et al., Biochimie, 75:49-54 (1993)], a phospholipid, e.g. di-hexadecyl-rac-glycerol or triethylammonium 1,2-di-O-hexadecyl-rac-glycero-3-H-phosphonate [Manoharan et al. Tetrahedron Lett., 36:3651-3654 (1995); Shea et al., Nucl. Acids Res., 18:3777-3783 (1990)], a polyamine or a polyethylene glycol chain [Manoharan et al., Nucleosides & Nucleotides, 14:969-973 (1995)], or adamantane acetic acid [Manoharan et al., Tetrahedron Lett., 36:3651-3654 (1995)], a palmityl moiety [Mishra et al., Biochim. Biophys. Acta, 1264:229-237 (1995)], or an octadecylamine or hexylamino-carbonyl-oxycholesterol moiety [Crooke et al., J. Pharmacol. Exp. Ther., 277:923-937 (1996)].

One skilled in the art will recognise that it is not necessary for all positions in a given oligonucleotide to be uniformly modified. The present invention, therefore, contemplates the incorporation of more than one of the aforementioned modifications into a single oligonucleotide or even at a single nucleoside within the oligonucleotide.

The present invention further includes antisense oligonucleotides that are chimeric oligonucleotides, i.e. oligonucleotides that contain two or more chemically distinct regions, each made up of at least one monomer unit. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression; Consequently, comparable results can often be obtained with shorter oligonucleotides when chimeric oligonucleotides are used, compared to phosphorothioate deoxyoligonucleotides hybridising to the same target region, Cleavage of the RNA target can be routinely detected by gel electrophoresis and, if necessary, associated nucleic acid hybridisation techniques known in the art.

In the context of the present invention, an antisense oligonucleotide is “nuclease resistant” when it has either been modified such that it is not susceptible to degradation by DNA and RNA nucleases or alternatively has been placed in a delivery vehicle which in itself protects the oligonucleotide from DNA or RNA nucleases. Nuclease resistant oligonucleotides include, for example, methyl phosphonates, phosphorothioates, phosphorodithioates, phosphotriesters, and morpholino oligomers. Suitable delivery vehicles for conferring nuclease resistance include, for example, liposomes. In one embodiment of the present invention, the antisense oligonucleotides are nuclease resistant.

The present invention further contemplates antisense oligonucleotides that contain groups for improving the pharmacokinetic properties of the oligonucleotide, or groups for improving the pharmacodynamic properties of the oligonucleotide,

### Preparation of the Antisense Oligonucleotides

The antisense oligonucleotides of the present invention can be prepared by conventional techniques well-known to those skilled in the art. For example, the oligonucleotides can be prepared using solid-phase synthesis using commercially available equipment, such as the equipment available from Applied Biosystems Canada Inc., Mississauga, Canada. As is well-known in the art, modified oligonucleotides, such as phosphorothioates and alkylated derivatives, can also be readily prepared by similar methods.

Alternatively, the antisense oligonucleotides of the present invention can be prepared by enzymatic digestion of the naturally occurring ribonucleotide reductase R2 gene by methods known in the art.

Antisense oligonucleotides can also be prepared through the use of recombinant methods in which expression vectors comprising nucleic acid sequences that encode the antisense oligonucleotides are expressed in a suitable host cell. Such expression vectors can be readily constructed using procedures known in the art. Examples of suitable vectors include, but are not limited to, plasmids, phagemids, cosmids, bacteriophages, baculoviruses and retroviruses, and DNA viruses. One skilled in the art will understand that selection of the appropriate host cell for expression of the antisense oligonucleotide will be dependent upon the vector chosen. Examples of host cells include, but are not limited to, bacterial, yeast, insect, plant and mammalian cells.

One skilled in the art will also understand that the expression vector may further include one or more regulatory elements, such as transcriptional elements, required for efficient transcription of the antisense oligonucleotide sequences. Examples of regulatory elements that can be incorporated into the vector include, but are not limited to, promoters, enhancers, terminators, and polyadenylation signals. One skilled in the art will appreciate that selection of suitable regulatory elements is dependent on the host cell chosen for expression of the antisense oligonucleotide and that such regulatory elements may be derived from a variety of sources, including bacterial, fungal, viral, mammalian or insect genes.

In accordance with the present invention, the expression vectors can be introduced into a suitable host cell or tissue by one of a variety of methods known in the art. Such methods can be found generally described in Sambrook et al., 1992; Ausubel et al., 1989; Chang et al., 1995; Vega et al., 1995; and Vectors: A Survey of Molecular Cloning Vectors and Their Uses (1988) and include, for example, stable or transient transfection, lipofection, electroporation, and infection with recombinant viral vectors.

### Immunotherapeutic Agents

The combination products of the present invention comprise one or more immunotherapeutic agents in combination with the antisense oligonucleotide against ribonucleotide reductase R2. Immunotherapy is a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art and contemplated for inclusion in the combination products of the present invention include, but are not limited to, cytokines, non-cytokine adjuvants, monoclonal antibodies and cancer vaccines.

Immunotherapeutic agents can be non-specific, i.e. boost the immune system generally so that it becomes more effective in fighting the growth and/or spread of cancer cells, or they can be specific, i.e. targeted to the cancer cells themselves immunotherapy regimens may combine the use of non-specific and specific immunotherapeutic agents. The combination products of the present invention can include an antisense oligonucleotides against ribonucleotide reductase R2 in combination with one or more non-specific immunotherapeutic agents, one or more specific immunotherapeutic agent, or combinations thereof. In one embodiment, the combination product comprises an antisense oligonucleotide in combination with one or more non-specific immunotherapeutic agents.

Non-specific immunotherapeutic agents are substances that stimulate or indirectly augment the immune system. Non-specific immunotherapeutic agents have been used alone as the main therapy for the treatment of cancer, as well as in addition to a main therapy, in which case he non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. In one embodiment of the present invvention, the combination product comprises one or more cytokine. Suitable cytolines for use in the combination therapies of the present invention include, but are not limited to, interferons, interleukins and colony-stimulating factors.

Interferons (IFNs) contemplated by the present invention for use in combination with the antisense oligonucleotides include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages.

In one embodiment of the present invention, the combination product comprises IFN-α. Recombinant IFN-α is available commercially as Roferon (Roche Pharmaceuticals) and Intron A (Schering Corporation). The use of IFN-α, alone or in combination with other immunotherapeutics or with chemotherapeutics, has shown efficacy in the treatment of various cancers including melanoma (including metastatic melanoma), renal cancer (including metastatic renal cancer), breast cancer, prostate cancer, cervical cancer (including metastatic cervical cancer), Kaposi's sarcoma, hairy cell leukemia, chronic myeloid leukemia (CML), multiple myeloma, follicular non-Hodgkin's lymphoma and cutaneous T cell lymphoma.

Interleukins contemplated by the present invention for use in combination with the antisense oligonucleotides include IL-2 (or aldesleukin), IL-4, IL-11 and IL-12 (or oprelvekin). Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Interleukins, alone or in combination with other immunotherapeutics or with chemotherapeutics, have shown efficacy in the treatment of various cancers including renal cancer (including metastatic renal cancer), melanoma (including metastatic melanoma), ovarian cancer (including recurrent ovarian cancer), cervical cancer (including metastatic cervical cancer), breast cancer, colorectal cancer, lung cancer, brain cancer, prostate cancer, leukemias and lymphomas.

In one embodiment of the present invention, the combination product comprises IL-2. Interleukins have also shown good activity in combination with IFN-α in the treatment of various cancers (Negrier et al., Ann Oncol. 2002 13(9):1460-8; Tourani et al., J Clin Oncol. 2003 21(21):3987-94). Accordingly, in another embodiment, the present invention provides for combination products that comprise one or more interleukins and IFN-α in combination with an antisense oligonucleotide against ribonucleotide reductase R2. In a further embodiment, the combination product comprises IL-2 and IFN-α in combination with an antisense oligonucleotide against ribonucleotide reductase R2.

An interleukin-immunotoxin conjugate known as denileukin diftitox (or Ontak; Seragen, Inc), which comprises IL-2 conjugated to diptheria toxin, has been approved by the FDA for the treatment of cutaneous T cell lymphoma and may also be included in the combination products of the present invention.

Colony-stimulating factors (CSFs) contemplated by the present invention for use in the combination products the antisense oligonucleotides include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in patients undergoing traditional chemotherapy. Accordingly, treatment with CSFs can be helpful in decreasing the side effects associated with chemotherapy and can allow for higher doses of chemotherapeutic agents to be used.

One embodiment of the present invention provides for a combination product comprising an antisense oligonucleotide against ribonucleotide reductase R2 and one or more CSFs. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Colony stimulating factors have shown efficacy in the treatment of cancer, including melanoma, colorectal cancer (including metastatic colorectal cancer), lung cancer and leukemia. The present invention further provides for the use of combination products comprising an antisense oligonucleotide and one or more CSFs in combination therapies together with higher than standard doses of a chemotherapeutic agent for the treatment of cancer.

In another embodiment of the present invention, the combination product comprises one or more non-cytokine adjuvants. Non-cytokine adjuvants suitable for use in the combinations of the present invention include, but are not limited to, Levamisole, alum hydroxide (alum), bacillus Calmette-Guerin (ACG), incomplete Freund's Adjuvant (IFA), QS-21, DETOX, Keyhole limpet hemocyanin (KLH) and dinitrophenyl (DNP). Non-cytokine adjuvants in combination with other immuno- and/or chemotherapeutics have demonstrated efficacy against various cancers including, for example, colon cancer and colorectal cancer (Levimasole); melanoma (BCG and QS-21); renal cancer and bladder cancer (BCG). Accordingly, a further embodiment of the present invention provides for combination products comprising an antisense oligonucleotide against ribonucleotide reductase R2 in combination with one or more non-cytokine adjuvants and an interferon. In another embodiment, the combination product comprises an antisense oligonucleotide against ribonucleotide reductase R2 in combination with Levamisole and IFN-α.

In addition to having specific or non-specific targets, immunotherapeutic agents can be active, i.e. stimulate the body's own immune response, or they can be passive, i.e. comprise immune system components that were generated external to the body. Both types of immunotherapeutic agents are suitable for use with the antisense oligonucleotides against ribonucleotide reductase R2 in the combination therapies of the present invention. In one embodiment, the antisense oligonucleotides are used in combination therapies with one or more active immunotherapeutic agents.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

In one embodiment, the present invention provides for combination products comprising one or more monoclonal antibodies in combination with an antisense oligonucleotide against ribonucleotide reductase R2 for the treatment of cancer. Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22.

Monoclonal antibodies are used in the treatment of a wide range of cancers including lymphomas (such as non-Hodgkin's lymphoma, B cell chronic lymphocytic leukemia (B-CLL)), myelomas (such as multiple myeloma), leukemias (such as B cell leukemia or acute myelogenous leukemia), breast cancer (including advanced metastatic breast cancer), colorectal cancer (including advanced and/or metastatic colorectal cancer), ovarian cancer, lung cancer, prostate cancer, cervical cancer, melanoma and brain tumours. Monoclonal antibodies can be used alone or in combination with other immunotherapeutic agents or chemotherapeutic agents.

Active specific immunotherapy typically involves the use of cancer vaccines. Cancer vaccines have been developed that comprise whole cancer cells, parts of cancer cells or one or more antigens derived from cancer cells. Cancer vaccines, alone or in combination with one or more immuno- or chemotherapeutic agents are being investigated in the treatment of several types of cancer including melanoma, renal cancer, ovarian cancer, breast cancer, colorectal cancer, lung cancer and leukemia Non-specific immunotherapeutics are useful in combination with cancer vaccines in order to enhance the body's immune response. One embodiment of the present invention provides for combination products comprising a cancer vaccine in combination with an antisense oligonucleotide against ribonucleotide reductase R2. The combination may further comprise one or more non-specific immunotherapeutic agents.

### Chemotherapeutic Agents

As indicated above, the combination products of the present invention can further comprise one or more chemotherapeutic agents. The chemotherapeutic agent(s) can be selected from a wide range of cancer chemotherapeutic agents known in the art. Known chemotherapeutic agents include those that are specific for the treatment of a particular type of cancer as well as those that are applicable to a range of cancers, such as doxorubicin, capecitabine, mitoxantrone, irinotecan (CPT-11), cisplatin and gemcitabine. Etoposide is generally applicable in the treatment of leukaemias (including acute lymphocytic leukaemia and acute myeloid leukaemia), germ cell tumours, Hodgkin's disease and various sarcomas. Cytarabine (Ara-C) is also applicable in the treatment of various leukaemias, including acute myeloid leukaemia, meningeal leukaemia, acute lymphocytic leukaemia, chronic myeloid leukaemia, erythroleukemia, as well as non-Hodgkin's lymphoma. Both types of chemotherapeutic agent are suitable for use in the combinations of the present invention.

Exemplary chemotherapeutics suitable for use in the combinations, which can be used for the treatment specific cancers, are provided in Table 2. One skilled in the art will appreciate that many other chemotherapeutics are available and that the following list is representative only.

TABLE 2
Exemplary Chemotherapeutics used in the Treatment of Some
Common Cancers

| CANCER | CHEMOTHERAPEUTIC | |
|---|---|---|
| Acute lymphocytic | Pegaspargase (e.g. Oncaspar ®) | L-asparaginase |
| leukaemia (ALL) | Cytarabine | |
| Acute myeloid | Cytarabine | Idarubicin |
| leukaemia (AML) | | |
| Brain cancer | Procarbazine (e.g. Matulane ®) | Nitrosoureas |
| | Platinum analogues | Temozolomide |
| Breast cancer | Capecitabine (e.g. Xeloda ®) | Cyclophosphamide |
| | 5-fluorouracil (5-FU) | Carboplatin |
| | Paclitaxel (e.g. Taxol ®) | Cisplatin |
| | Docetaxel (e.g. Taxotere ®) | Ifosfamide |
| | Epi-doxorubicin (epirubicin) | Doxorubicin (e.g. Adriamycin ®) |
| | Tamoxifen | |
| Chronic myeloid | Cytarabine | |
| leukaemia (CML) | | |
| Colon cancer | Edatrexate (10-ethyl-10-deaza-aminopterin) | |
| | Methyl-chloroethyl-cyclohexyl-nitrosourea | |
| | 5-fluorouracil (5-FU) | Oxaliplatin |
| | Fluorodeoxyuridine (FUdR) | Vincristine |
| | Capecitabine (e.g. Xeloda ®) | |
| Colorectal cancer | Irinotecan (CPT-11, e.g. Camptosar ®) | |
| | Loperamide (e.g. Imodium ®) | |
| | Topotecan (e.g. Hycamtin ®) | Methotrexate |
| | Capecitabine (e.g. Xeloda ®) | Oxaliplatin |
| | 5-fluorouracil (5-FU) | |
| Gall bladder | 5-fluorouracil (5-FU) | |
| Genitourinary | Docetaxel (e.g. Taxotere ®) | |
| cancer | | |
| Head and neck | Docetaxel (e.g. Taxotere ®) | Cisplatin |
| cancer | | |
| Non-Hodgkin's | Procarbazine (e.g. Matulane ®) | Cytarabine |
| Lymphoma | Etoposide | |
| Non-small-cell lung | Vinorelbine Tartrate (e.g. Navelbine ®) | |
| (NSCL) cancer | Irinotecan (CPT-11, e.g. Camptosar ®) | |
| | Docetaxel (e.g. Taxotere ®) | Paclitaxel (e.g. Taxol ®) |
| | Gemcitabine (e.g. Gemzar ®) | Topotecan |
| Oesophageal cancer | Porfimer Sodium (e.g. Photofrin ®) | |
| | Cisplatin | |
| Ovarian cancer | Irinotecan (CPT-11, e.g. Camptosar ®) | |
| | Topotecan (e.g. Hycamtin ®) | |
| | Docetaxel (e.g. Taxotere ®) | Paclitaxel (e.g. Taxol ®) |
| | Gemcitabine (e.g. Gemzar ®) | Amifostine (e.g. Ethyol ®) |
| Pancreatic cancer | Irinotecan (CPT-11, e.g. Camptosar ®) | |
| | Gemcitabine (e.g. Gemzar ®) | 5-fluorouracil (5-FU) |
| Promyelocytic | Tretinoin (e.g. Vesanoid ®) | |
| leukaemia | | |
| Prostate cancer | Goserelin Acetate (e.g. Zoladex ®) | |
| | Mitoxantrone (e.g. Novantrone ®) | |
| | Prednisone (e.g. Deltasone ®) | Liarozole |
| | Nilutamide (e.g. Nilandron ®) | Flutamide (e.g. Eulexin ®) |
| | Finasteride (e.g. Proscar ®) | Terazosin (e.g. Hytrin ®) |
| | Doxazosin (e.g. Cardura ®) | Cyclophosphamide |
| | Docetaxel (e.g. Taxotere ®) | Estramustine |
| | Luteinizing hormone releasing hormone agonist | |
| Renal cancer | Capecitabine (e.g. Xeloda ®) | |
| | Gemcitabine (e.g. Gemzar ®) | |
| Small cell lung | Cyclophosphamide | Vincristine |
| cancer | Doxorubicin | Etoposide |
| Solid tumours | Gemicitabine (e.g. Gemzar ®) | Cyclophosphamide |
| | Capecitabine (e.g. Xeloda ®) | Ifosfamide |
| | Paclitaxel (e.g. Taxol ®) | Cisplatin |
| | Docetaxel (e.g. Taxotere ®) | Carboplatin |
| | Epi-doxorubicin (epirubicin) | Doxorubicin (e.g. Adriamycin ®) |
| | 5-fluorouracil (5-FU) | |

As indicated above, combinations of chemotherapeutics may be employed. Combination therapies using standard cancer chemotherapeutics are well known in the art and may be included as part of the combinations of the invention. Exemplary combination therapies include for the treatment of breast cancers the combination of epirubicin with paclitaxel or docetaxel, or the combination of doxorubicin or epirubicin with cyclophosphamide. Polychemotherapeutic regimens are also useful and may consist, for example, of doxorubicin/cyclophosphamide/5-fluorouracil or cyclophosphamide/epirubicin/5-fluorouracil. Many of the above combinations are useful in the treatment of a variety of other solid tumours.

Combinations of etoposide with either cisplatin or carboplatin are used in the treatment of small cell lung cancer. In the treatment of stomach or oesophageal cancer, combinations of doxorubicin or epirubicin with cisplatin and 5-fluorouracil are useful. For colorectal cancer, CPT-11 in combination with 5-fluorouracil-based drugs, or oxaliplatin in combination with 5-fluorouracil-based drugs can be used. Oxaliplatin may also be used in combination with capecitabine.

Other examples include the combination of cyclophosphamide, doxorubicin, vincristine and prednisone in the treatment of non-Hodgkin's lymphoma; the combination of doxorubicin, bleomycin, vinblastine and dacarbazine (DTIC) in the treatment of Hodgkin's disease and the combination of cisplatin or carboplatin with any one, or a combination, of gemcitabine, paclitaxel, docetaxel, vinorelbine or etoposide in the treatment of non-small cell lung cancer.

Various sarcomas are treated by combination therapy, for example, for osteosarcoma combinations of doxorubicin and cisplatin or methotrexate with leucovorin are used; for advanced sarcomas etoposide can be used in combination with ifosfamide; for soft tissue sarcoma doxorubicin or dacarbazine can be used alone or, for advanced sarcomas doxorubicin can be used in combination with ifosfamide or dacarbazine, or etoposide in combination with ifosfamide.

Ewing's sarcoma/peripheral neuroectodermal tumour (PNET) or rhabdomyosarcoma can be treated using etoposide and ifosfamide, or a combination of vincristine, doxorubicin and cyclophosphamide.

The alkylating agents cyclophosphamide, cisplatin and melphalan are also often used in combination therapies with other chemotherapeutics in the treatment of various cancers.

Retinoic acid and its derivatives have been demonstrated to have efficacy against some forms of cancer, notably lung, breast, head and neck, and blood cancers. The retinoic acid derivative, Vesanoid® (tretinoin; all trans-retinoic acid), has been approved by the FDA for patients with acute promyelocytic leukemia (APL). 13-cis retinoic acid or all-trans retinoic acid in combination with IFN-α have also been shown to have efficacy against renal cell carcinoma and 13-cis retinoic acid in combination with IL-2 has shown efficacy in the treatment of recurrent ovarian cancer. Accordingly, the present invention contemplates that 13-cis retinoic acid or all-trans retinoic acid may be included in the combinations of the invention.

Specific combinations of immunotherapeutic agents and chemotherapeutic agents that may be included with an antisense oligonucleotide against ribonucleotide reductase R2 in the combination products of the present invention include, but are not limited to, IFN-α and vinblastine, IFN-α and 5-FU, IFN-α and 13-cis retinoic acid, IFN-α and all-trans retinoic acid, IL-2 and 5-FU, IL-2 and 13-cis retinoic acid, IL-2 plus IFN-α and 5-FU.

### Efficacy of the Combinations of the Invention

The combinations of antisense oligonucleotides and one or more immunotherapeutic agents can be tested in vitro and in vivo using standard techniques. Exemplary methods are described below and in the Examples provided herein.

### 1. In Vitro Testing

Initial determinations of the ability of the combinations to attenuate the growth or proliferation of neoplastic cells may be made using in vitro techniques if required.

For example, the cytotoxicity of the combinations can be assayed in vitro using a suitable cancer cell line. In general, cells of the selected test cell line are grown to an appropriate density and the test compound(s) are added. After an appropriate incubation time (for example, about 48 to 72 hours), cell survival is assessed; Methods of determining cell survival are well known in the art and include, but are not limited to, the resazurin reduction test (see Fields & Lancaster (1993) Am. Biotechnol. Lab. 11:48-50; O'Brien et al., (2000) Eur. J. Biochem. 267:5421-5426 and U.S. Pat. No. 5,501,959), the sulforhodamine assay (Rubinstein et al., (1990) J. Natl. Cancer Inst. 82:113-118) or the neutral red dye test (Kitano et al., (1991) Euro. J. Clin. Investg. 21:53-58; West et al., (1992) J. Investigative Derm. 99:95-100). Cytotoxicity is determined by comparison of cell survival in the treated culture with cell survival in one or more control cultures, for example, untreated cultures, cultures pre-treated with a control compound (typically a known therapeutic) and/or cultures treated individually with the components of the combination.

Alternatively, the ability of the combinations to inhibit proliferation of neoplastic cells can be assessed by culturing cells of a cancer cell line of interest in a suitable medium. After an appropriate incubation time, the cells can be treated with the combination and incubated for a further period of time. Cells are then counted and compared to an appropriate control as described above.

The combinations can also be tested in vitro by determining their ability to inhibit anchorage-independent growth of tumour cells. Anchorage-independent growth is known in the art to be a good indicator of tumorigenicity. In general, anchorage-independent growth is assessed by plating cells from an appropriate cancer cell-line onto soft agar and determining the number of colonies formed after an appropriate incubation period. Growth of cells treated with the combinations can then be compared with that of cells treated with an appropriate control (as described above) and with that of untreated cells.

A variety of cancer cell-lines suitable for testing the combinations are known in the art and many are commercially available (for example, from the American Type Culture Collection, Manassas, Va.). In one embodiment of the present invention, in vitro testing of the combinations is conducted in a human cancer cell-line. Examples of suitable cancer cell-lines for in vitro testing include, but are not limited to, breast cancer cell-lines MDA-MB-231 and MCF-7, renal carcinoma cell-line A-498, mesothelial cell lines MSTO-211H, NCI-H2052 and NCI-H28, ovarian cancer cell-lines OV90 and SK-OV-3, colon cancer cell-lines CaCo, HCT116 and HT29, cervical cancer cell-line HeLa, non-small cell lung carcinoma cell-lines A549 and H1299, pancreatic cancer cell-lines MIA-PaCa-2 and AsPC-1, prostatic cancer-cell line PC-3, bladder cancer cell-liie T24, liver cancer cell-lineHepG2, brain cancer cell-line U-87 MG, melanoma cell-line A2058, lung cancer cell-line NCI-H460. Other examples of suitable cell-lines are known in the art.

If necessary, the toxicity of the combinations can also be initially assessed in vitro using standard techniques. For example, human primary fibroblasts can be treated in vitro with the oligonucleotide in the presence of a commercial lipid carrier such as lipofectamine. Cells are then tested at different time points following treatment for their viability using a standard viability assay, such as the trypan-blue exclusion assay. Cells are also assayed for their ability to synthesize DNA, for example, using a thymidine incorporation assay, and for changes in cell cycle dynamics, for example, using a standard cell sorting assay in conjunction with a fluorocytometer cell sorter (FACS).

### 2. In Vivo Testing

The ability of the combinations to inhibit tumour growth or proliferation in vivo can be determined in an appropriate animal model using standard techniques known in the art (see, for example, Enna, et al., Current Protocols in Pharmacology, J. Wiley & Sons, Inc., New York, N.Y.).

In general, current animal models for screening anti-tumour compounds are xenograft models, in which a human or mammalian tumour has been implanted into an animal. Examples of xenograft models of human cancer include, but are not limited to, human solid tumour xenografts in mice, implanted by sub-cutaneous injection and used in tumour growth assays; human solid tumour isografts in mice, implanted by fat pad injection and used in tumour growth assays; human solid tumour orthotopic xenografts, implanted directly into the relevant tissue and used in tumour growth assays; experimental models of lymphoma and leukaemia in mice, used in survival assays, and experimental models of metastasis in mice.

For example, the combinations can be tested in vivo on solid tumours using mice that are subcutaneously grafted bilaterally with a predetermined amount of a tumour fragment on day 0. The animals bearing tumours are mixed before being subjected to the various treatments and controls. In the case of treatment of advanced tumours, tumours are allowed to develop to the desired size, animals having insufficiently developed tumours being eliminated. The selected animals are distributed at random into groups that will undergo the treatments or act as controls. Suitable groupings would be, for example, those receiving the combination of the invention, those receiving the antisense alone, those receiving the anticancer agent(s) alone and those receiving no treatment. Animals not bearing tumours may also be subjected to the same treatments as the tumour-bearing animals in order to be able to dissociate the toxic effect from the specific effect on the tumour. Treatment generally begins from 3 to 22 days after grafting, depending on the type of tumour, and the animals are observed every day. The combinations of the present invention can be administered to the animals, for example, by bolus infusion. The different animal groups are weighed about 3 or 4 times a week until the maximum weight loss is attained, after which the groups are weighed less frequently, for example, at least once a week until the end of the trial.

The tumours are measured about 2 or 3 times a week until the tumour reaches a pre-determined size and/or weight, or until the animal dies if this occurs before the tumour reaches the pre-determined size/weight. The animals are then sacrificed and the tissue histology, size and/or proliferation of the tumour assessed.

For the study of the effect of the compositions on leukaemias, the animals are grafted with a particular number of cells, and the anti-tumour activity is determined by the increase in the survival time of the treated mice relative to the controls.

To study the effect of the combinations of the present invention on tumour metastasis, tumour cells are typically treated with the composition ex vivo and then injected into a suitable test animal. The spread of the tumour cells from the site of injection is then monitored over a suitable period of time by standard techniques.

In vivo toxic effects of the oligonucleotides can be evaluated by measuring their effect on animal body weight during treatment and by performing haematological profiles and liver enzyme analysis after the animal has been sacrificed.

TABLE 3
Examples of xenograft models of human cancer

| Cancer Model | Cell Type |
|---|---|
| Tumour Growth Assay | Prostate (PC-3, DU145) |
| Human solid tumour xenografts | Breast (MDA-MB-231, MVB-9) |
| in mice (sub-cutaneous | Colon (HT-29) |
| injection) | Lung (NCI-H460, NCI-H209) |
| | Pancreatic (ASPC-1, SU86.86) |
| | Pancreatic: drug resistant (BxPC-3) |
| | Skin (A2058, C8161) |
| | Cervical (SIHA, HeLa-S3) |
| | Cervical: drug resistant (HeLa S3-HU- |
| | resistance) |
| | Liver (HepG2) |
| | Brain (U87-MG) |
| | Renal (Caki-1, A498) |
| | Ovary (SK-OV-3) |
| Tumour Growth Assay | Breast: drug resistant (MDA-CDDP-S4, |
| Human solid tumour isografts in | MDA-MB435-To.1) |
| mice (fat pad injection) | |
| Survival Assay | Human: Burkitts lymphoma (Non- |
| Experimental model of | Hodgkin's) (raji) |
| lymphoma and leukaemia | Murine: erythroleukemia (CB7 Friend |
| in mice | retrovirus-induced) |
| Experimental model of lung | Human: melanoma (C8161) |
| metastasis in mice | Murine: fibrosarcoma (R3) |

In one embodiment of the present invention, the combinations comprising the antisense oligonucleotide with one or more immunotherapeutic agents are more effective than each of the components when used alone. Improved efficacy can be manifested, for example, as a less-than additive effect, wherein the effect of the combination is greater than the effect of each component alone, but less than the sum of the effects of the components, or it may be an additive effect, wherein the effect of the combination is equivalent to the sum of the effects of the components when used individually, or it may be a more-than-additive effect, wherein the effect of the combination is greater than the sum of the effects of each component used alone. Greater than additive effects may also be described as synergistic. The improved efficacy of the combinations can be determined by a number of methods known in the art.

For example, such improved efficacy can result in one or more of: (i) an increase in the ability of the combination to inhibit the growth or proliferation of neoplastic cells when compared to the effect of each component alone; (ii) a decrease in the dose of one or more of the components being required to bring about a certain effect (i.e. a decrease in the median effective dose or ED₅₀); (iii) decreased toxicity phenomena associated with one or more of the components (i.e. a increase in the median lethal dose or LD₅₀), and (iv) an improved therapeutic index or clinical therapeutic index of the combination when compared to the therapeutic index/clinical therapeutic index of each component alone.

As used herein, the term “therapeutic index” is defined as ID₅₀/ED₅₀, where “ED₅₀” is the amount of a compound that produces 50% of the maximum response or effect associated with the compound, or the amount that produces a predetermined response or effect in 50% of a test population, and “LD₅₀” is the amount of a compound that has a lethal effect in 50% of a test population. Thus, a compound with a high therapeutic index can typically be administered with greater safety than one with a low therapeutic index. The LD₅₀ is determined in preclinical trials, whereas the ED₅₀ can be determined in preclinical or clinical trials. Preclinical trials are conducted using an appropriate animal model, such as those described herein. The therapeutic index can also be determined based on doses that produce a therapeutic effect and doses that produce a toxic effect (for example, the ED₉₀ and LD₁₀, respectively).

“Clinical therapeutic index” differs from therapeutic index in that some indices of relative safety or relative effectiveness in patients in a clinical setting cannot be defined explicitly and uniquely. A combination is considered to demonstrate an improved Clinical Therapeutic Index, therefore, when it meets one of the following criteria as defined by the Food and Drug Administration: demonstrates increased safety (or patient acceptance) at an accepted level of efficacy within the recommended dosage range, or demonstrates increased efficacy at equivalent levels of safety (or patient acceptance) within the recommended dosage range, as compared to each of the components in the combination. Alternatively, during clinical studies, the dose or the concentration (for example, in solution, blood, serum, plasma) of a drug required to produce toxic effects can be compared to the concentration required to achieve the desired therapeutic effects in the population in order to evaluate the clinical therapeutic index. Methods of clinical studies to evaluate the clinical therapeutic index are well known to workers skilled in the art.

In another embodiment of the present invention, the combinations comprising the antisense oligonucleotide with one or more immunotherapeutic agents exhibit therapeutic synergy, wherein “therapeutic synergy” is demonstrated when a combination is therapeutically superior to one of the components of the combination when used at that component's optimum dose [as defined in T. H. Corbett et al., (1982) Cancer Treatment Reports, 66:1187]. To demonstrate the efficacy of a combination, it may be necessary to compare the maximum tolerated dose of the combination with the maximum tolerated dose of each of the separate components in the study in question. This efficacy may be quantified using techniques and equations commonly known to workers skilled in the art [see, for example, T. H. Corbett et al., (1977) Cancer, 40, 2660.2680; F. M. Schabel et al., (1979) Cancer Drug Development, Part B, Methods in Cancer Research, 17:3-51, New York, Academic Press Inc.].

### Use of the Combinations of the Present Invention

The combinations of the present invention can be used in the treatment of a variety of cancers. In accordance with one embodiment of the present invention, the combination is more effective in the treatment of cancer than either the antisense oligonucleotide or the immunotherapeutic agent(s) alone. The present invention contemplates that treatment of cancer encompasses the use of the combinations to treat, stabilize or prevent cancer. In this context, treatment with the combinations may result in a reduction in the size of a tumour, the slowing or prevention of an increase in the size of a tumour, an increase in the disease-free survival time between the disappearance or removal of a tumour and its reappearance, prevention of an initial or subsequent occurrence of a tumour (e.g. metastasis), an increase in the time to progression, reduction of one or more adverse symptom associated with a tumour, a slowing of tumour regression, or an increase in the overall survival time of a subject having cancer.

One embodiment of the present invention provides for the treatment of a patient having cancer with a combination comprising an antisense oligonucleotide against ribonucleotide reductase and one or more immunotherapeutic agents resulting in a increased time to progression (TTP), a reduction of one or more adverse symptom associated with the cancer, a slowing of tumour regression, or an increase in the overall survival time of the patient.

Examples of cancers which may be may be treated or stabilized in accordance with the present invention include, but are not limited to haematologic neoplasms, including leukaemias and lymphomas; carcinomas, including adenocarcinomas; melanomas and sarcomas. Carcinomas, adenocarcinomas and sarcomas are also frequently referred to as “solid tumours,” examples of commonly occurring solid tumours include, but are not limited to, cancer of the brain, breast, cervix, colon, head and neck, kidney, lung, ovary, pancreas, prostate, stomach and uterus, non-small cell lung cancer and colorectal cancer. Various forms of lymphoma also may result in the formation of a solid tumour and, therefore, in certain contexts may also be considered to be solid tumours.

The term “leukaemia” refers broadly to progressive, malignant diseases of the blood-forming organs. Leukaemia is typically characterized by a distorted proliferation and development of leukocytes and their precursors in the blood and bone marrow but can also refer to malignant diseases of other blood cells such as erythroleukemia, which affects immature red blood cells. Leukaemia is generally clinically classified on the basis of (1) the duration and character of the disease—acute or chronic; (2) the type of cell involved—myeloid (myelogenous), lymphoid (lymphogenous) or monocytic, and (3) the increase or non-increase in the number of abnormal cells in the blood—leukaemic or aleukaemic (subleukaemic). Leukaemia includes, for example, acute nonlymphocytic leukaemia, chronic lymphocytic leukaemia, acute granulocytic leukemia, chronic granulocytic leukaemia, acute promyelocytic leukaemia, adult T-cell leukaemia, aleukaemic leukaemia, aleukocythemic leukaemia, basophylic leukaemia, blast cell leukaemia, bovine leukaemia, chronic myelocytic leukaemia, leukaemia cutis, embryonal leukaemia, eosinophilic leukaemia, Gross' leukaemia, hairy-cell leukaemia, hemoblastic leukaemia, hemocytoblastic leukaemia, histiocytic leukaemia, stem cell leukaemia, acute monocytic leukaemia, leukopenic leukaemia, lymphatic leukaemia, lymphoblastic leukaemia, lymphocytic leukaemia, lymphogenous leukaemia, lymphoid leukaemia, lymphosarcoma cell leukaemia, mast cell leukaemia, megakaryocytic leukaemia, micromyeloblastic leukaemia, monocytic leukaemia, myeloblastic leukaemia, myelocytic leukaemia, myeloid granulocytic leukaemia, myelomonocytic leukaemia, Naegeli leukaemia, plasma cell leukaemia, plasmacytic leukaemia, promyelocytic leukaemia, Rieder cell leukaemia, Schilling's leukaemia, stem cell leukaemia, subleukaemic leukaemia, and undifferentiated cell leukaemia.

The term “lymphoma” generally refers to a malignant neoplasm of the lymphatic system, including cancer of the lymphatic system. The two main types of lymphoma are Hodgkin's disease (HD or HL) and non-Hodgkin's lymphoma (NHL). Abnormal cells appear as congregations which enlarge the lymph nodes, form solid tumours in the body, or more rarely, like leukemia, circulate in the blood. Hodgkin's disease lymphomas, include nodular lymphocyte predominance Hodgkin's lymphoma; classical Hodgkin's lymphoma; nodular sclerosis Hodgkin's lymphoma; lymphocyte-rich classical Hodgkin's lymphoma; mixed cellularity Hodgkin's lymphoma; lymphocyte depletion Hodgkin's lymphoma. Non-Hodgkin's lymphomas include small lymphocytic NHL, follicular NHL; mantle cell NHL; mucosa-associated lymphoid tissue (MALT) NHL; diffuse large cell B-cell NHL; mediastinal large B-ell NHL; precursor T lymphoblastic NHL; cutaneous T-cell NHL; T-cell and natural killer cell NHL; mature (peripheral) T-cell NHL; Burkitt's lymphoma; mycosis fungoides; Sézary Syndrome; precursor B-lymophoblastic lymphoma; B-cell small lymphocytic lymphoma; lymphoplasmacytic lymphoma; spenic marginal zome B-cell lymphoma; nodal marginal zome lymphoma; plasma cell myeloma/plasmacytoma; intravascular large B-cell NHL; primary effusion lymphoma; blastic natural killer cell lymphoma; enteropathy-type T-cell lymphoma; hepatosplenic gamma-delta T-cell lymphoma; subcutaneous panniculitis-like T-cell lymphoma; angioimmunoblastic T-cell lymphoma; and primary systemic anaplastic large T/null cell lymphoma.

The term “sarcoma” generally refers to a tumour which originates in connective tissue, such as muscle, bone, cartilage or fat, and is made up of a substance like embryonic connective tissue and is generally composed of closely packed cells embedded in a fibrillar or homogeneous substance. Sarcomas include soft tissue sarcomas, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumour sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented haemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumour arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma; basosquamous cell carcinoma, bronchioalveolar carcinoma, bronchiolar carcinoma, bronchogenic carcinoma, cerebriform carcinoma, cholangiocellular carcinoma, chorionic carcinoma, colorectal carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, haematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, non-small cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “carcinoma” also encompasses adenocarcinomas. Adenocarcinomas are carcinomas that originate in cells that make organs which have glandular (secretory) properties or that originate in cells that line hollow viscera, such as the gastrointestinal tract or bronchial epithelia. Examples include, but are not limited to, adenocarcinomas of the breast, lung, pancreas and prostate.

Additional cancers encompassed by the present invention include, for example, multiple myeloma, neuroblastoma, rhabdomyosarcoma, primary thrombocytosis, primary macroglobutlinemia, small-cell lung tumours, primary brain tumours, malignant pancreatic insulanoma, malignant carcinoid, urinary bladder cancer, premalignant skin lesions, gliomas, testicular cancer, thyroid cancer, esophageal cancer, genitourinary tract cancer, malignant hypercalcemia, endometrial cancer, adrenal cortical cancer, mesothelioma and medulloblastoma.

As demonstrated herein, antisense oligonucleotides against ribonucleotide reductase R2 have been shown to be effective against a wide range of cancers including lymphomas, leukemias and solid tumours, all of which can also benefit from treatment with immunotherapeutic agents. One embodiment of the present invention, therefore, provides for the use of the combinations in the treatment of a lymphoma, leukemia or solid tumour. In another embodiment, the present invention provides for the use of the combinations in the treatment of Burkitt's lymphoma, erythroleukemia, acute myeloid leukemia, promyelocytic leukemia, or a solid cancer selected from the group of colon cancer, colorectal cancer, renal cancer, prostate cancer, melanoma, breast cancer, ovarian cancer, pancreatic cancer, cervical cancer and lung cancer. In a further embodiment, the present invention provides for the use of the combinations in the treatment of a cancer that has been shown to respond to immunotherapy, including solid tumours such as melanoma, renal cancer, breast cancer, prostate cancer, cervical cancer, ovarian cancer, colon cancer, colorectal cancer, lung cancer, brain cancer and recurrent and metastatic versions thereof; Kaposi's sarcoma; multiple myeloma; lymphomas such as follicular non-Hodgkin's lymphoma and cutaneous T cell lymphoma; and leukemias such as hairy cell leukemia and chronic myeloid leukemia (CML).

### Combination Therapies

The combinations of the present invention are administered to a subject in an amount effective to achieve the intended purpose. The exact dosage to be administered can be readily determined by the medical practitioner, in light of factors related to the patient requiring treatment. Dosage and administration are adjusted to provide sufficient levels of each component of the combination and/or to maintain the desired effect, Factors which may be taken into account when determining an appropriate dosage include the severity of the disease state, general health of the subject, age, weight, and gender of the subject, diet, time and frequency of administration, the particular components of the combination, reaction sensitivities, and tolerance/response to therapy.

Antisense oligonucleotides are typically adminstered parenterally, for example, by intravenous infusion. Other methods of administering antisense oligonucleotides are known in the art. Methods of administering standard immunotherapeutic agents are also known in the art and include subcutaneous injection, and intravenous, intramuscular or intrasternal injection or infusion techniques.

In general, determination of the severity of disease requires identification of certain disease characteristics, for example, whether the cancer is pre-metastatic or metastatic, the stage and/or grade of cancer, and the like.

Staging is a process used to describe how advanced a cancer is in a subject. Staging can be important in determining a prognosis, planning treatment and evaluating the results of such treatment. While different cancer staging systems may need to be used for different types of cancer, most staging systems generally involve describing how far the cancer has spread anatomically and attempt to put subjects with similar prognosis and treatment in the same staging group.

Examples of common staging systems used for most solid tumours, some leukemias and lymphomas are the Overall Stage Grouping system and the TMN system. In the Overall Stage Grouping system, Roman numerals I through IV are utilized to denote the four stages of a cancer. Generally, if a cancer is only detectable in the area of the primary lesion without having spread to any lymph nodes it is called Stage I. Stage II and III cancers are generally locally advanced and/or have spread to the local lymph nodes. For example, if the cancer is locally advanced and has spread only to the closest lymph nodes, it is called Stage II. In Stage III, the cancer is locally advanced and has generally spread to the lymph nodes in near proximity to the site of the primary lesion. Cancers that have metastasized from the primary tumour to a distant part of the body, such as the liver, bone, brain or another site, are called, Stage IV, the most advanced stage. Accordingly, stage I cancers are generally small localized cancers that are curable, while stage IV cancers usually represent inoperable or metastatic cancers. As with other staging systems, the prognosis for a given stage and treatment often depends on the type of cancer, for example, a stage II non small cell lung cancer has a different prognosis and treatment than a stage II cervical cancer. For some cancers, classification into four prognostic groups is insufficient and the overall staging is further divided into subgroups, for example, with classifications such as IIIa and IIIb. In contrast, some cancers may have fewer than four stage groupings. Additionally, a cancer that recurs after all visible tumour has been eradicated is called recurrent disease, with local recurrence occurring in the location of the primary tumour and distant recurrence representing distant metastasis. Under this staging system, stage IV can be used interchangeably with distant recurrence.

In the TMN system, the type of tumour, is indicated by T; regional lymph node involvement, is indicated by N; and distant metastases, is indicated by M. Each of the T, N and M categories are classified separately with a number, to determine the total stage. The T category classifies the extent of the primary tumour and is given a number from T0 through to T4, for example, T0 represents a primary tumour that has not invaded the local tissues (e.g. in situ), while T4 represents a large primary tumour that may have invaded other organs and is likely inoperable. The N category classifies whether the cancer has metastasized to nearby lymph nodes and is given a number from N0 through to N4, for example, N0 means no lymph node involvement while N4 indicates extensive involvement. The definition of which lymph nodes are regional may depend on the type of cancer. The M category classifies distant metastases and is given a number of M0 or M1, for example, M0 means no distant metastases and M1 indicates distant metastases. As with other staging systems, the exact definition for T and N may differ for each different kind of cancer.

As described above, variations to the staging systems may depend on the type of cancer. For example, most leukemias do not have a staging system as they are not anatomically localized like other solid primary tumours, although a few forms of leukemia do have staging systems to describe the degree of disease advancement. A few leukemias can be defined in stages from I through to IV, but these stages depend, for example, on various factors such as the blood count, extent of bone marrow involvement or the presence or absence of symptoms. Moreover, certain types of cancers, such as prostate cancer or colon cancer, may use staging systems with different nomenclatures, for example, the Duke staging system for colon cancer. The staging system for individual cancers may be revised with new information and subsequently, the resulting stage may change the prognosis and treatment for a specific cancer.

The “grade” of a cancer is used to describe how closely a tumour resembles normal tissue of its same type. Based on the microscopic appearance of a tumour, pathologists identify the grade of a tumour based on parameters such as cell morphology, cellular organization, and other markers of differentiation. As a general rule, the grade of a tumour corresponds to its rate of growth or aggressiveness and tumours are typically classified from the least aggressive (Grade I) to the most aggressive (Grade IV). Accordingly, the higher the grade, the more aggressive and faster growing the cancer. Information about tumour grade is useful in planning treatment and predicting prognosis.

The American Joint Commission on Cancer has recommended the following guidelines for grading tumours: 1) GX Grade cannot be assessed; 2) G1 Well differentiated; G2 Moderately well differentiated; 3) G3 Poorly differentiated; 4) G4 Undifferentiated. Although grading is used by pathologists to describe most cancers, it plays a more important role in treatment planning for certain types of cancers than for others. An example is the Gleason system that is specific for prostate cancer, which uses grade numbers to describe the degree of differentiation (low scores indicate well differentiated cells and high scores describe poorly differentiated cells). Grade is also important in some types of brain tumours and soft tissue sarcomas.

In accordance with the present invention, the combinations can be used to treat various stages and grades of cancer development and progression. The present invention, therefore, contemplates the use of the combinations in the treatment of early stage cancers including early neoplasias that may be small, slow growing, localized and/or nonaggressive, for example, with the intent of curing the disease or causing regression of the cancer, as well as in the treatment of intermediate stage and in the treatment of late stage cancers including advanced and/or metastatic and/or aggressive neoplasias, for example, to slow the progression of the disease, to reduce metastasis or to increase the survival of the patient. Similarly, the combinations may be used in the treatment of low grade cancers, intermediate grade cancers and or high grade cancers.

The present invention also contemplates that the combinations can be used in the treatment of indolent cancers, recurrent cancers including locally recurrent, distantly recurrent and/or refractory cancers (i.e. cancers that have not responded to treatment), metastatic cancers, locally advanced cancers and aggressive cancers.

One skilled in the art will appreciate that many of these categories may overlap, for example, aggressive cancers are typically also metastatic. “Aggressive cancer,” as used herein, refers to a rapidly growing cancer. One skilled in the art will appreciate that for some cancers, such as breast cancer or prostate cancer the term “aggressive cancer” will refer to an advanced cancer that has relapsed within approximately the earlier two-thirds of the spectrum of relapse times for a given cancer, whereas for other types of cancer, such as small cell lung carcinoma (SCLC) nearly all cases present rapidly growing cancers which are considered to be aggressive. The term can thus cover a subsection of a certain cancer type or it may encompass all of other cancer types.

Antisense oligonucleotides against ribonucleotide reductase R2 have also been demonstrated to be effective against drug-resistant tumour cells. Accordingly, the combinations may also be used to treat drug-resistant cancers, including multidrug resistant tumours. As is known in the art, the resistance of cancer cells to chemotherapy is one of the central problems in the management of cancer.

Certain cancers, such as prostate and breast cancer, can be treated by hormone therapy, i.e. with hormonal agents including, for example, hormones or anti-hormone drugs that slow or stop the growth of certain cancers by blocking the body's natural hormones. Such cancers may develop resistance, or be intrinsically resistant, to hormone therapy. The present invention further contemplates the use of the combinations in the treatment of such “hormone-resistant” or “hormone-refractory” cancers.

It is contemplated that the combinations of the present invention can be used alone or in combination with one or more immunotherapeutic agents as part of a primary therapy or an adjuvant therapy. “primary therapy” or “first-line therapy” refers to treatment upon the initial diagnosis of cancer in a subject. Exemplary primary therapies may involve surgery, a wide range of chemotherapies, immunotherapy and radiotherapy. When first-line or primary therapy is not systemic chemotherapy or immunotherapy, then subsequent chemotherapy or immunotherapy may be considered as “first-line systemic therapy.” In one embodiment of the present invention, the combinations are used for first-line systemic therapy.

“Adjuvant therapy” refers to a therapy that follows a primary therapy and that is administered to subjects at risk of relapsing. Adjuvant systemic therapy is typically begun soon after primary therapy to delay recurrence, prolong survival or cure a subject. Treatment of a refractory cancer may be termed a “second-Line therapy” and is a contemplated use of the present invention, in addition to first-line therapy.

In one embodiment of the present invention, the combinations are used in the treatment of an early stage cancer. In another embodiment, the combinations are used as a first-line systemic therapy for an early stage cancer.

In an alternate embodiment, the combinations are used in the treatment of a late stage and/or advanced and/or metastatic cancer. In another embodiment, the combinations are used in the treatment of a Grade III or Grade IV tumour. In a further embodiment, the combinations are adminstered as a first-line systemic therapy for the treatment of a late stage and/or advanced and/or metastatic cancer.

In a specific embodiment of the present invention, the late stage and/or advanced and/or metastatic cancer is a solid tumour. In a further embodiment, the solid tumour is melanoma, renal cancer, breast cancer, prostate cancer, cervical cancer, ovarian cancer, colon cancer, colorectal cancer, lung cancer, or brain cancer.

### Pharmaceutical Compositions

The antisense oligonucleotide(s) may be administered as a pharmaceutical composition with an appropriate pharmaceutically physiologically acceptable carrier, diluent, excipient or vehicle. The pharmaceutical compositions may also be formulated to contain the antisense olignucleotide and one or more immunotherapeutic agents for concurrent administration to a patient.

The pharmaceutical compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase may be a vegetable oil, for example, olive oil or arachis oil, or a mineral oil, for example, liquid paraffin, or it may be a mixtures of these oils. Suitable emulsifying agents may be naturally-occurring gums, for example, gum acacia or gum tragacanth; naturally-occurring phosphatides; for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

The pharmaceutical compositions may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are, sterile, fixed oils which are conventionally employed as a solvent or suspending medium, and a variety of bland fixed oils including, for example, synthetic mono- or diglycerides. In addition, fatty acids such as oleic acid find use in the preparation of injectables.

In one embodiment of the present invention, the pharmaceutical composition comprising the antisense oligonucleotide is formulated for injection or infusion.

Other pharmaceutical compositions and methods of preparing pharmaceutical compositions are known in the art and are described, for example, in “Remington: The Science and Practice of Pharmacy,” Gennaro, A., Lippincott, Williams & Wilkins, Philidelphia, Pa. (2000) (formerly “Remingtons Pharmaceutical Sciences”).

### Clinical Trials in Cancer Patients

One skilled in the art will appreciate that, following the demonstrated effectiveness of the combinations of the present invention in vitro and in animal models, they should be tested in Clinical Trials in order to further evaluate their efficacy in the treatment of cancer and to obtain regulatory approval for therapeutic use. As is known in the art, clinical trials progress through phases of testing, which are identified as Phases I, II, III, and IV, Representative examples of Phase I/II Clinical Trials are provided in the Examples herein.

Initially the combinations will be evaluated in a Phase I trial. Typically Phase I trials are used to determine the best mode of administration (for example, by pill or by injection), the frequency of administration, and the toxicity for the compounds. Phase I studies frequently include laboratory tests, such as blood tests and biopsies, to evaluate the effects of a compound in the body of the patient. For a Phase I trial, a small group of cancer patients are treated with a specific dose of the antisense oligonucleotide and the one or more immunotherapeutic(s). During the trial, the dose is typically increased group by group in order to determine the maximum tolerated dose (MTD) and the dose-limiting toxicities (DLT) associated with the compound. This process determines an appropriate dose to use in a subsequent Phase II trial.

A Phase II trial can be conducted to evaluate further the effectiveness and safety of the combinations. In Phase II trials, the combination is administered to groups of patients with either one specific type of cancer or with related cancers, using the dosage found to be effective in Phase I trials.

Phase III trials focus on determining how a compound compares to the standard, or most widely accepted, treatment. In Phase III trials, patients are randomly assigned to one of two or more “arms”. In a trial with two arms, for example, one arm will receive the standard treatment (control group) and the other arm will receive treatment with the combination of the present invention (investigational group).

Phase IV trials are used to further evaluate the long-term safety and effectiveness of a compound. Phase IV trials are less common than Phase I, II and III trials and will take place after the combination has been approved for standard use.

### Eligibility of Patients for Clinical Trials

Participant eligibility criteria can range from general (for example, age, sex, type of cancer) to specific (for example, type and number of prior treatments, tumour characteristics, blood cell counts, organ function). Eligibility criteria may also vary with trial phase. For example, in Phase I and II trials, the criteria often exclude patients who may be at risk from the investigational treatment because of abnormal organ function or other factors. In Phase II and III trials additional criteria are often included regarding disease type and stage, and number and type of prior treatments.

Phase I cancer trials usually comprise 15 to 30 participants for whom other treatment options have not been effective. Phase II trials typically comprise up to 100 participants who have already received chemotherapy, surgery, or radiation treatment, but for whom the treatment has not been effective. Participation in Phase II trials is often restricted based on the previous treatment received. For trials that are investigating the use of the combinations of the invention as a first line therapy, for example, the patients selected for participation should not have undergone any prior systemic therapy. Phase III trials usually comprise hundreds to thousands of participants. This large number of participants is necessary in order to determine whether there are true differences between the effectiveness of the combination of the present invention and the standard treatment. Phase III may comprise patients ranging from those newly diagnosed with cancer to those with extensive disease in order to cover the disease continuum.

One skilled in the art will appreciate that clinical trials should be designed to be as inclusive as possible without making the study population too diverse to determine whether the treatment might be as effective on a more narrowly defined population. The more diverse the population included in the trial, the more applicable the results could be to the general population, particularly in Phase III trials. Selection of appropriate participants in each phase of clinical trial is considered to be within the ordinary skills of a worker in the art.

### Assessment of Patients Prior to Treatment

Prior to commencement of the study, several measures known in the art can be used to first classify the patients. Patients can first be assessed, for example, using the Eastern Cooperative Oncology Group (ECOG) Performance Status (PS) scale. ECOG PS is a widely accepted standard for the assessment of the progression of a patient's disease as measured by functional impairment in the patient, with ECOG PS 0 indicating no functional impairment, ECOG PS 1 and 2 indicating that the patients have progressively greater functional impairment but are still ambulatory and ECOG PS 3 and 4 indicating progressive disablement and lack of mobility.

Patients' overall quality of life can be assessed, for example, using the McGill Quality of Life Questionnaire (MQOL) (Cohen et al (1995) Palliative Medicine 9: 207-219). The MQOL measures physical symptoms; physical, psychological and existential well-being; support; and overall quality of life. To assess symptoms such as nausea, mood, appetite, insomnia, mobility and fatigue the Symptom Distress Scale (SDS) developed by McCorkle and Young ((1978) Cancer Nursing 1: 373-378) can be used.

Patients can also be classified according to the type and/or stage of their disease and/or by tumour size.

### Administration of the Combinations of the Present Invention in Clinical Trials

The antisense oligonucleotide and the one or more immunotherapeutic agent(s) are typically administered to the trial participants parenterally. In one embodiment, the combination is administered by intravenous infusion. Methods of administering drugs by intravenous infusion are known in the art. Usually intravenous infusion takes place over a certain time period, for example, over the course of 60 minutes.

### Monitoring of Patient Outcome

The endpoint of a clinical trial is a measurable outcome that indicates the effectiveness of a treatment under evaluation. The endpoint is established prior to the commencement of the trial and will vary depending on the type and phase of the clinical trial. Examples of endpoints include, for example, tumour response rate—the proportion of trial participants whose tumour was reduced in size by a specific amount, usually described as a percentage; disease-free survival—the amount of time a participant survives without cancer occurring or recurring, usually measured in months; overall survival—the amount of time a participant lives, typically measured from the beginning of the clinical trial until the time of death. For advanced and/or metastatic cancers, disease stabilisation—the proportion of trial participants whose disease has stabilised, for example, whose tumour(s) has ceased to grow and/or metastasise (“progress”), can be used as an endpoint. Other endpoints include toxicity and quality of life.

Tumour response rate is a typical endpoint in Phase II trials. However, even if a treatment reduces the size of a participant's tumour and lengthens the period of disease-free survival, it may not lengthen overall survival. In such a case, side effects and failure to extend overall survival might outweigh the benefit of longer disease-free survival. Alternatively, the participant's improved quality of life during the tumour-free interval might outweigh other factors. Thus, because tumour response rates are often temporary and may not translate into long-term survival benefits for the participant, response rate is a reasonable measure of a treatment's effectiveness in a Phase II trial, whereas participant survival and quality of life are typically used as endpoints in a Phase III trial.

### Pharmaceutical Kits

The present invention additionally provides for therapeutic kits containing the antisense oligonucleotide and one or more immunotherapeutic agents for use in the treatment of cancer. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. In this case the container means may itself be an inhalant, syringe, pipette, eye dropper, or other such like apparatus, from which the composition may be administered to a patient.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or any such medically approved delivery vehicle.

To gain a better understanding of the invention described herein, the following examples are set forth. It should be understood that these examples are for illustrative purposes only. Therefore, they should not limit the scope of this invention in any way.

### EXAMPLES

SEQ ID NO:1 as referred to in the following Examples 1 to 15 is a fully phosphorothioated oligonucleotide with the sequence:

| | |
|---|---|
| 5′-GGCTAAATCGCTCCACCAAG-3′ | [SEQ ID NO:1] |

SEQ ID NO:1 hybridizes to the coding region of human ribonucleotide reductase R2 mRNA.

SEQ ID NO:2 is a mismatched control oligonucleotide for SEQ ID NO:1, having four base changes in the middle of the sequence with respect to SEQ ID NO:1:

| | |
|---|---|
| 5′-GGCTAAACTCGTCCACCAAG-3′ | [SEQ.ID NO:2] |

SEQ ID NO:3 is a scrambled control oligonucleotide for SEQ ID NO:1. SEQ ID NO:3 is not complementary to of human ribonucleotide reductase R2 mRNA, but retains the same base composition ratio as SEQ ID NO:1.

| | |
|---|---|
| 5′-ACGCACTCAGCTAGTGACAC-3′ | [SEQ ID NO:3] |

The phosphorothioates were synthesized on an automated DNA synthesizer (Perkin-Elmer, USA) by Boston BioSystem Inc. (Boston, Mass.) and were purified by reversed-phase high performance liquid chromatography.

SEQ ID NO:1 is currently being studied in several clinical trials for the treatment of various cancers in combination with standard chemotherapeutic agents as described herein.

### Example 1

### In vitro Testing of Interferon Alpha (IFN Alpha) in Human Renal Carcinoma Cell Lines

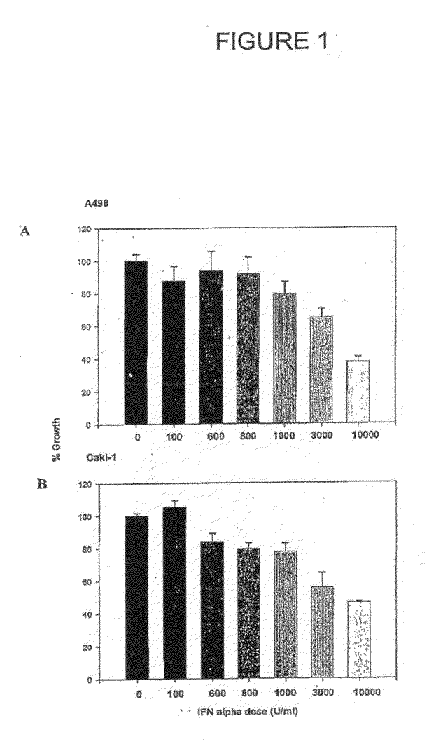

Preliminary in vitro testing was performed on human renal carcinoma cell lines (A498 and Caki-1) to determine whether these cell lines were sensitive to the direct anti-proliferative effects of IFN alpha. Cultured cells were treated for 96 hours with increasing concentrations of IFN alpha (0, 100, 600, 800, 1000, 3000, or 10000 U/ml) and cell proliferation was assessed by XTT assay. The in vitro anti-proliferative effects of IFN alpha are shown in FIG. 1A (A498) and FIG. 1B (Caki-1), which illustrates that both cell lines were sensitive to IFN alpha in a dose-dependent manner.

### Example 2

### In vivo Testing of SEQ ID NO:1 in Combination with Interferon Alpha in a Mouse Xenograft Model of Human Renal Carcinoma (Caki-1) #1

Cell Line: Human renal carcinoma cell line (Caki-1) was grown as monolayer culture in Minimum essential medium (α-MEM) supplemented with 10% fetal bovine serum (FEBS), 0.1 mM non-essential amino acid, 1.0 mM sodium pyruvate, 100 U/ml penicillin, 100 μg/ml streptomycin and 0.25 μg/ml amphotericin B and 2 mM L-alanyl-L-glutamine at 37° C. in an atmosphere of 5% CO₂ in air. The tumour cells were routinely subcultured twice weekly by trypsin-EDTA treatment. The cells were harvested from subconfluent logarithmically growing culture by treatment with trypsin-EDTA and counted for tumour inoculation.

Tumour Inoculation: An acclimation period of at least 7 days was allowed between animal receipt and commencement of tumour inoculation. When the female SCID mice were 6-7 weeks of age (20-25 g), each mouse was subcutaneously (s.c.) injected with 5×10⁶ Caki-1 human renal carcinoma cells in 0.1 ml of PBS at the right flank of the mice to induce tumour growth.

Dose-Preparation and Treatment: The dose for SEQ ID NO:1 was formulated using the following procedure. From a concentrated stock solution (usually prepared in ddH₂O at a concentration of 50 mg/ml and kept at −80° C.), SEQ ID NO:1 was diluted with saline to achieve a final target concentration of 1 or 2.5 mg/ml on the first day of dosing. Enough SEQ ID NO:1 was diluted so that the compound could be administered by bolus infusion into the tail vein every other day for the duration of the experiment. Recombinant interferon alfa-2b (Intron A™) was utilized according to the dose and treatment schedules below. The treatments were started 7 days after the tumour cell inoculation when the tumour size reached an approximate volume of 125 mm³. Each treatment group contained 10 tumour-bearing mice. Routes of administration are indicated as follows: intravenous (i.v.) and intratumoural (i:t.).

Groups and Treatments: The following treatment groups were evaluated:
1. Saline treated group-1: saline 0.1 ml/mouse/48 hours, i.v., n=10
2. Saline treated group-2: saline 0.05 ml/mouse/5 days/week, i.t., n=10
3. SEQ ID NO:1 treated group: 1 mg/kg/48 hours in 0.1 ml saline, i.v. n=10
4. SEQ ID NO:1 treated group: 2.5 mg/kg/48 hours in 0.1 ml saline, i.v., n=10
5. Intron A 10³ unit/5 days/week treated group: 10³ unit Intron A in 0.05 ml saline, i.t., n=10
6. Intron A 10⁵ unit/5 days/week treated group: 10⁵ unit Intron A in 0.05 ml saline, i.t., n=10
7. SEQ ID NO:1 1 mg/kg Plus Intron A 10³ treated group: “3+5”, n=10
8. SEQ ID NO:1 1 mg kg Plus Intron A 10⁵ treated group: “3+6”, n=10
9. SEQ ID NO:1 2.5 mg/kg Plus Intron A 10⁵ treated group: “4+5”, n=10
10. SEQ ID NO:1 2.5 mg/kg Plus Intron A 10⁵ treated group: “4+6”, n=10

Mice received 25 treatments with SEQ ID NO:1 and 30 treatments with Intron A. The dosing schedule for Intron A was: treatment with Intron A for 3 consecutive weeks, 1 week off, and treatment with Intron A again for another 3 consecutive weeks. The dosing schedule for SEQ ID NO:1 was treatment with SEQ ID NO:1 every other day for the duration of the experiment.

Endpoints: The effects of SEQ ID NO:1 and Intron A on tumour growth were determined. Tumour sizes were measured every week from day 7 after the tumour cell inoculation in two dimensions using a caliper, and the volume was expressed in mm³ using the formula: V=0.5 a×b², where a and b are the long and short diameters of the tumour, respectively. Mean tumour volumes calculated from each measurement were then plotted in a standard graph to compare the anti-tumour efficacy to that of controls. A day after the last treatment, tumours were excised from the animals and their weights were measured. A standard bar graph was used to demonstrate the differences in tumour weights with each bar representing mean tumour weight calculated from 10 animals.

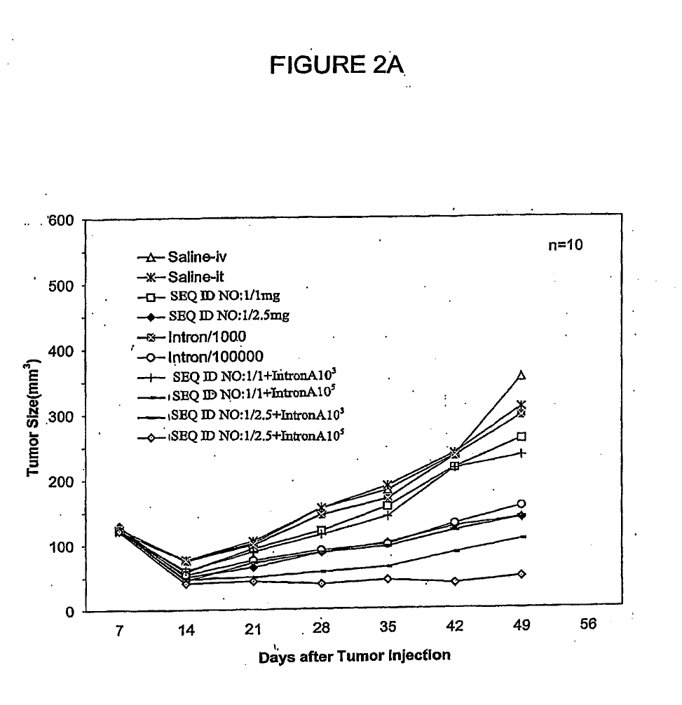

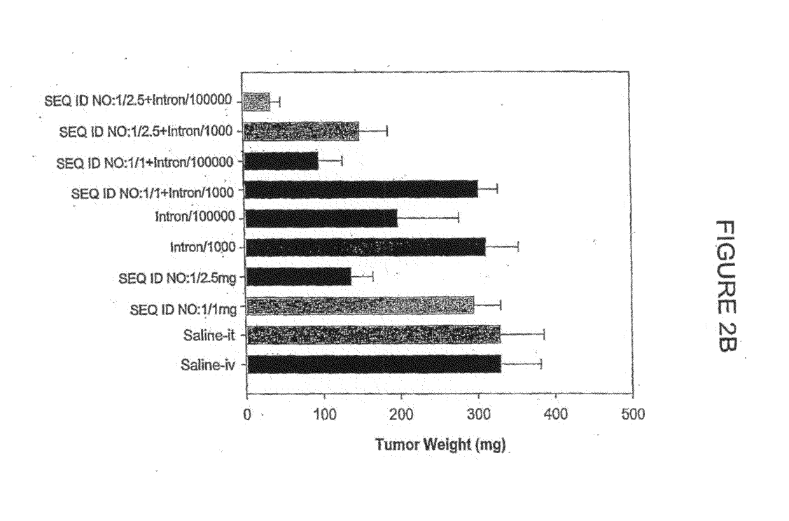

The results of the tumour volume at different time points after tumour inoculation are shown in FIG. 2A and the tumour weights at the experimental endpoint are shown in FIG. 2B, illustrating that SEQ ID NO:1 and Intron A have significant anti-tumour efficacy as monotherapies and when in combination, the effect is at least additive. Notably, even at sub-optimal drug dosages the combination effect remains at least additive, indicating potential synergy. The results from the combination treatments of SEQ ID NO:1 and Intron A at high dose combination (2.5 mg/kg and 10⁵, respectively) demonstrated dramatic regression of renal cell tumours in mice. A total of 5 mice in this group demonstrated total regression and the tumours in the other 5 treated animals did not grow beyond 100 mg, showing partial regression and stabilization.

### Example 3

### In vivo Testing of SEQ ID NO:1 in Combination with Interferon Alpha in a Mouse Xenograft Model of Human Renal Carcinoma (Caki-1) #2

A second independent in vivo study was conducted as described in Example 1 utilizing SCID mice (female, 6 weeks old) and Caki-1 (human renal carcinoma cell line) to test the efficacy of SEQ ID NO:1 in combination with IFN alpha Briefly, 5×10⁶ Caki-1 human renal carcinoma cells in 0.1 ml of PBS were injected subcutaneously at the right flank of the mice to induce tumour growth. Treatments started on the seventh day after tumour cell injection. Mice received a total of 23 treatments with SEQ ID NO:1 and 35 treatments with Intron A. Routes of administration are indicated as follows: intravenous (i.v.) and intratumoural (i.t).

### Groups and Treatment:

1. Saline treated group-A: saline 0.1 ml/mouse/every 2 days, i.v., n=10
2. Saline treated group-B: saline 0.05 ml/mouse/5 days/week, i.t., n=10.
3. SEQ ID NO:1 treated group: 2.5 mg/kg/every 2 days in 0.1 ml saline, i.v. n=10
4. SEQ ID NO:1 treated group: 5 mg/kg/every 2 days in 0.1 ml saline, i.v., n=10
5. Intron 10⁵ unit/5 days/week treated group: 10⁵ unit Intron in 0.05 ml saline, i.t., n=10
6. SEQ ID NO:12.5 mg/kg Plus Intron 10⁵ treated group: “3.+5.”, n=10
7. SEQ ID NO:15 mg/kg Plus Intron 10⁵ treated group; “4, +5.”, n=10

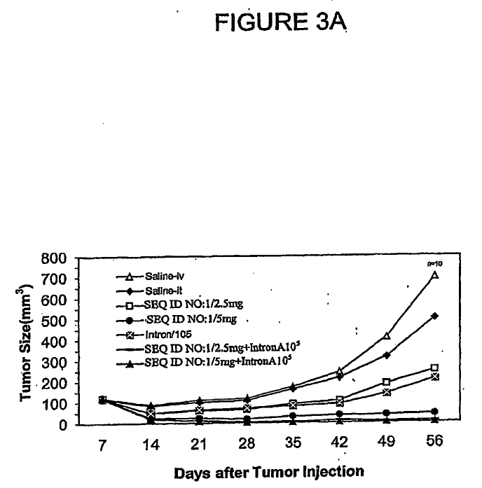

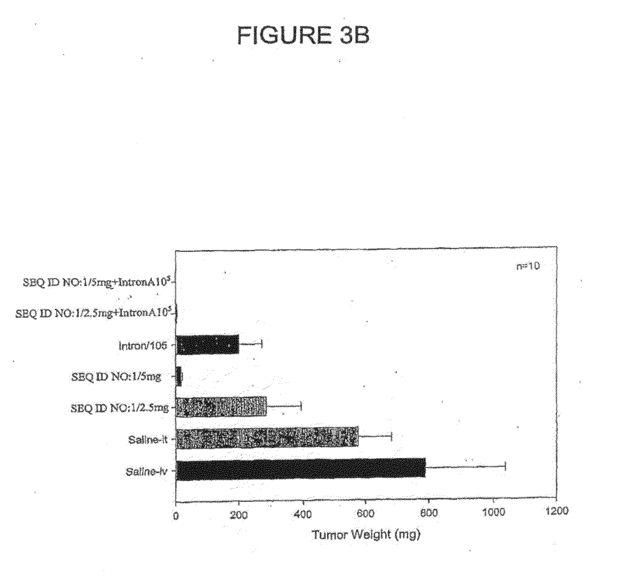

The results of the tumour volume at different time points after tumour inoculation are shown in FIG. 3A and the tumour weights at the experimental endpoint are shown in FIG. 3B, illustrating that SEQ ID NO:1 and Intron A have significant anti-tumour efficacy as monotherapies and in combination the effect is at least additive.

### Example 4

### In vivo Testing of SEQ ID NO: 1 in Combination with Interferon Alpha in a Mouse Xenograft Model of Human Renal Carcinoma (Caki-1) #3

A third independent in vivo study was conducted as described in Example 1 utilizing SCID mice (female, 6 weeks old) and Caki-1 (human renal carcinoma cell line) to test the efficacy of SEQ ID NO:1 in combination with IFN alpha. Briefly, 5×10⁶ Caki-1 human renal carcinoma cells in 0.1 ml of PBS were injected subcutaneously at the right flank of the mice to induce tumour growth. Treatments started on the seventh day after tumour cell injection. Mice received a total of 23 treatments with SEQ ID NO:1 and 35 treatments with Intron A; Routes of administration are indicated as follows: intravenous (i.v) and intratumoural (i.t.).

### Groups and Treatment:

1. Saline treated group: saline 0.1 ml/mouse/every 2 days, i.v., n=10
2. SEQ ID NO:1 treated group: 2.5 mg/kg/eyery 2 days in 0.1 ml saline, i.v. n=10
3. SEQ ID NO:3 treated group: 2.5 mg/kg/every 2 days in 0.1 ml saline, i.v. n=10
4. SEQ ID NO:1 plus Intron A 10⁵ treated group: “2.+5.”, n=10
5. Intron A 10⁵ unit/5 days/week treated group: 10 unit Intron A in 0.05 ml saline, i.t., n=10
6. SEQ ID NO:3 Plus Intron A 105 treated group: “3.+5.”, n=10

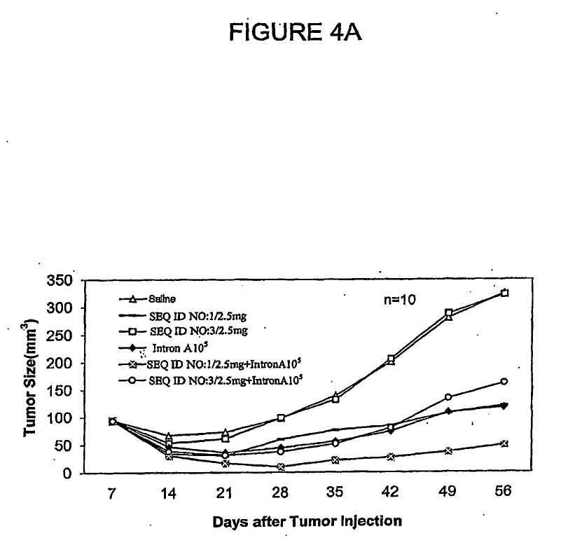

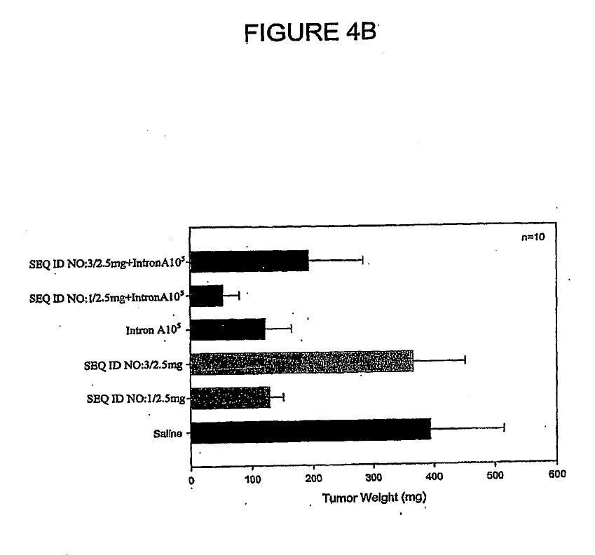

The results of the tumour volume at different time points after tumour inoculation are shown in FIG. 4A and the tumour weights at the experimental endpoint are shown in FIG. 4B, illustrating that both SEQ ID NO:1 and Intron A have significant anti-tumour efficacy as monotherapies and in combination the effect is at least additive. In contrast, the scrambled control oligonucleotide (SEQ ID NO:3) had no efficacy as a monotherapy and did not improve the efficacy of Intron A.

### Example 5

### In vivo Testing of SEQ ID NO:1 in Combination with Interferon Alpha in a Mouse Xenograft Model of Human Renal Carcinoma (Caki-1) #4

A fourth independent in vivo study was conducted as described in Example 1 utilizing SCID mice and Caki-1 (human renal carcinoma cell line) to test the efficacy of SEQ ID NO:1 in combination with IFN alpha. In a clinical setting, IFN alpha is typically administered subcutaneously. Accordingly, the effectiveness of SEQ ID NO:1 in combination with IFN alpha administered subcutaneously and with IFN alpha administered intratumourally was compared. Routes of administration are as follows: intravenous (i.v.), subcutaneous (s.c.) and intratumoural (i.t.).

### Groups and Treatments (Ten Mice Per Group):

1. treated with 0.1 ml saline solution per 48 hours, i.v. (n=10)
2. treated with 2.5 mg/kg/2 days SEQ ID NO:1 in 0.1 ml saline, i.v. (n=10).
3. treated with 100000 units Intron A/mouse/day×3/week in 50 μl saline, i.t. (n=10)
4. treated with 2.5 mg/kg/2 days SEQ ID NO:1 in 0.1 ml saline, i.v. plus 100000 units Intron A/mouse/day×3/week in 50 ul saline, i.t. (n=10)
5. treated with 100000 units Intron A/mouse/day×3/week, s.c. (n=10)
6. treated with 2.5 mg/kg/2 days SEQ ID NO:1 in 0.1 ml saline, i.v. plus 100000 units Intron A/mouse/day×3/week in 50 μl saline, s.c. (n=10)

Treatment started from day 7 after inoculation of human renal tumour cells.

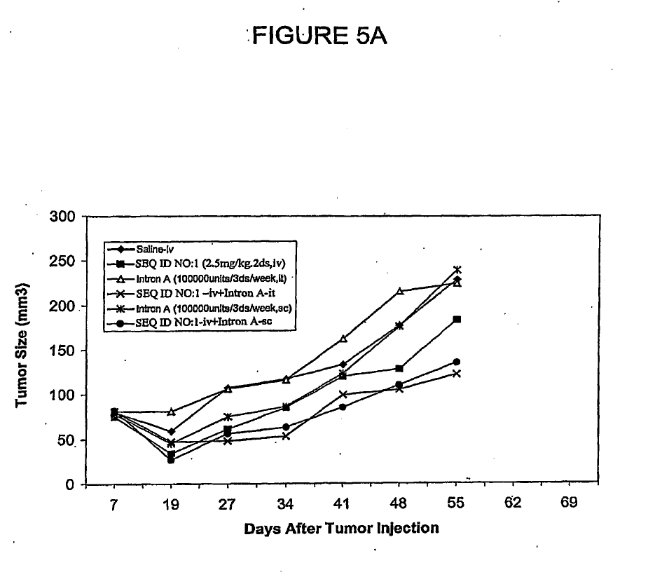

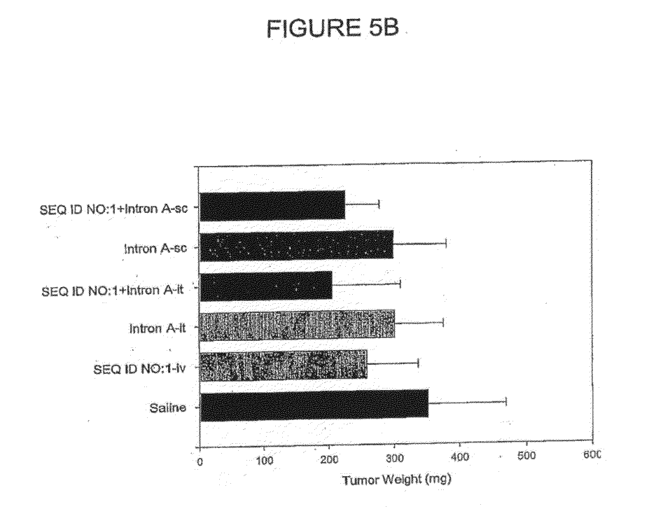

The results demonstrating the tumour volume at different time points after tumour inoculation are shown in FIG. 5A and the tumour weights at the experimental endpoint are shown in FIG. 5B. These results clearly show that Intron A is equally effective in combination with SEQ ID NO:1 whether administered subcutaneously or intratumourally.

### Example 6

### In vivo Testing of SEQ ID NO:1 in Combination with Interferon Alpha in a Mouse Xenograft Model of Human Renal Carcinoma (A498) #1

Cell line: Human renal carcinoma cell line (A498) was grown as monolayer culture in Minimum essential medium (α-MEM) supplemented with 10% fetal bovine serum (FBS), 0.1 mM non-essential amino acid, 1.0 mM sodium pyruvate, 100 U/ml penicillin, 100 μg/ml streptomycin and 0.25 μg/ml amphotericin B and 2 mM L-alanyl-1-glutamine at 37° C. in an atmosphere of 5% CO₂ in air. The tumour cells were routinely subcultured twice weekly by trypsin-EDTA treatment. The cells were harvested from subconfluent logarithmically growing culture by treatment with trypsin-EDTA and counted for tumour inoculation.

Tumour Inoculation: An acclimation period of at least 7 days was allowed between animal receipt and commencement of tumour inoculation. When the female SCID mice were 6-7 weeks of age (20-25 g), each mouse was subcutaneously injected with 9×10⁶ A498 human renal carcinoma cells in 0.1 ml of PBS at the right flank of the mice to induce tumour growth.

Dose Preparation and Treatment: The dose for SEQ ID NO:1 was formulated using the following procedure. From a concentrated stock solution (usually prepared in ddH₂O at a concentration of 50 mg/ml and kept at −80° C.), SEQ ID NO:1 was diluted with saline to achieve a final target concentration of 1 or 2.5 mg/ml on the first day of dosing. Enough SEQ ID NO:1 was diluted so that the compound could be administered by bolus infusion into the tail vein every other day for the duration of the experiment. Recombinant interferon alfa-2b (ntron A™) was employed according to the dose and treatment schedules below. The treatments were started 20 days after the tumour cell inoculation when the tumour size reached an approximate volume of 200 mm³. Each treatment group contained 10 tumour-bearing mice. Routes of administration are as follows: intravenous (i.v.) and intratumoural (i.t.).

### Groups and Treatments:

1. Saline treated group-1: saline 0.1 ml/mouse/48 hours, i.v., n=10
2. Saline treated group-2: saline 0.05 ml/mouse/5 days/week, i.t., n=10
3. SEQ ID NO:1 treated group: 1 mg/kg/48 hours in 0.1 ml saline, i.v. n=10
4. SEQ ID NO:1 treated group: 2.5 mg/kg/48 hours in 0.1 ml saline, i.v., n=10
5. Intron A 10³ unit/5 days/week treated group: 10³ unit Intron A in 0.05 ml saline, i.t., n=10
6. Intron A 10⁵ unit/5 days/week treated group: 10⁵ unit Intron A in 0.05 ml saline, i.t., n=10
7. SEQ ID NO:1 1 mg/kg Plus Intron A 10³ treated group: “3+5”, n=10
8. SEQ ID NO:1 1 mg/kg Plus Intron A 10⁵ treated group: “3+6”, n=10
9. SEQ ID NO:1 2.5 mg/g Plus Intron A 10³ treated group: “4+5”, n=10
10. SEQ ID NO:1 2.5 mg/kg Plus Intron A 10⁵ treated group: “4+6”, n=10

Treatment started from day 20 after inoculation of human renal tumour cells. Mice received 15 treatments with SEQ ID NO:1 and 12 treatments with Intron A. The dosing schedule for Intron A was: treatment with Intron A for 2 consecutive weeks plus treatment for 2 additional days and off for the rest of the experiment. The dosing schedule for SEQ ID NO:1 was treatment with SEQ ID NO:1 every other day for the duration of the experiment.

Endpoints: The effects of SEQ ID NO:1 and Intron A on tumour growth was determined. Tumour sizes were measured every week from day 20 after the tumour cell inoculation in two dimensions using a caliper, and the volume was expressed in mm³ using the formula: V=0.5 a×b², where a and b are the long and short diameters of the tumour, respectively. Mean tumour volumes calculated from each measurement were then plotted in a standard graph to compare the anti-tumour efficacy to that of controls. A day after the last treatment, tumours were excised from the animals and their weights were measured. A standard bar graph was used to demonstrate the differences in tumour weights with each bar representing mean tumour weight calculated from 10 animals.

The results of the tumour volume at different time points after tumour inoculation are shown in FIG. 6A and the tumour weights at the experimental endpoint are shown in FIG. 6B, illustrating that both SEQ ID NO:1 and Intron A have significant anti-tumour efficacy as monotherapies and in combination, the effect is at least additive. Notably, even at sub-optimal drug dosages the combination effect remains at least additive, suggesting potential synergy. The results from the combination treatments of SEQ ID NO:1 and Intron A at high dose combination (2.5 mg/kg and 10⁵ respectively) demonstrated dramatic regression of renal cell tumours in mice. All 10 mice in this group demonstrated total regression of all tumours.

### Example 7

### In vivo Testing of SEQ ID NO:1 in Combination with Interferon Alpha in a Mouse Xenograft Model of Human Renal Carcinoma (A498) #2

A second independent in vivo study was conducted as described in Example 6 utilizing SCID mice (female, 6 weeks old) and the A498 cell line (human renal carcinoma) to test the efficacy of SEQ ID NO:1 in combination with IFN alpha Briefly, 9×10⁶ Caki-1 human renal carcinoma cells in 0.1 ml of PBS were injected subcutaneously at the right flank of the mice to induce tumour growth. Treatments started from day 20 after tumour cell injection. Routes of administration are as follows: intravenous (i.v.) and intratumoural (i.t.).

### Groups and Treatments:

Group 1: treated with 0.1 ml saline per 48 hours, i.v. (n=10)
Group 2: treated with 50 ul saline per day/week×5, i.t. (n=10)
Group 3: treated with 10000 units Intron A/mouse/day×5/week for 2 weeks in 50 ul saline, i.t. (n=10)
Group 4: treated with 2.5 mg/kg/2ds SEQ ID NO:1 in 0.1 ml saline, i.v. (n=10)
Group 5: treated with 2.5 mg/kg/2ds SEQ ID NO:1, i.v. plus 10000 units Intron A/mouse/5ds/week for 2 weeks, i.t. (n=10).

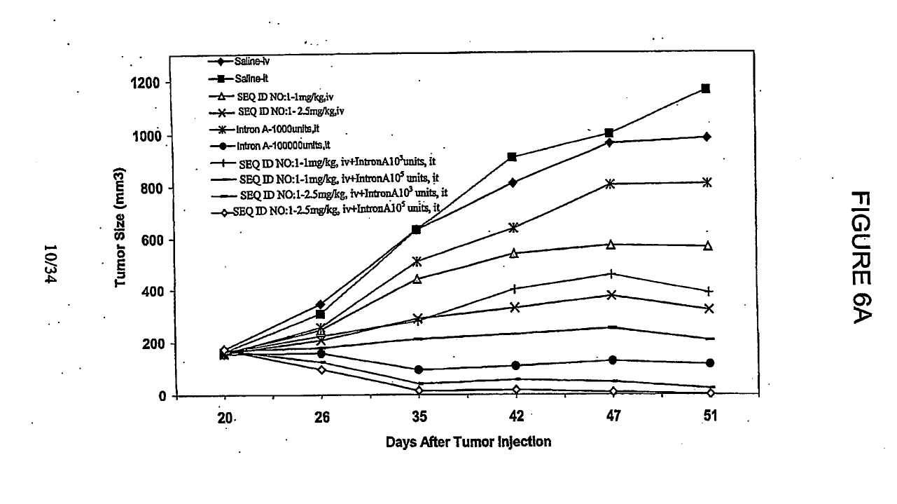

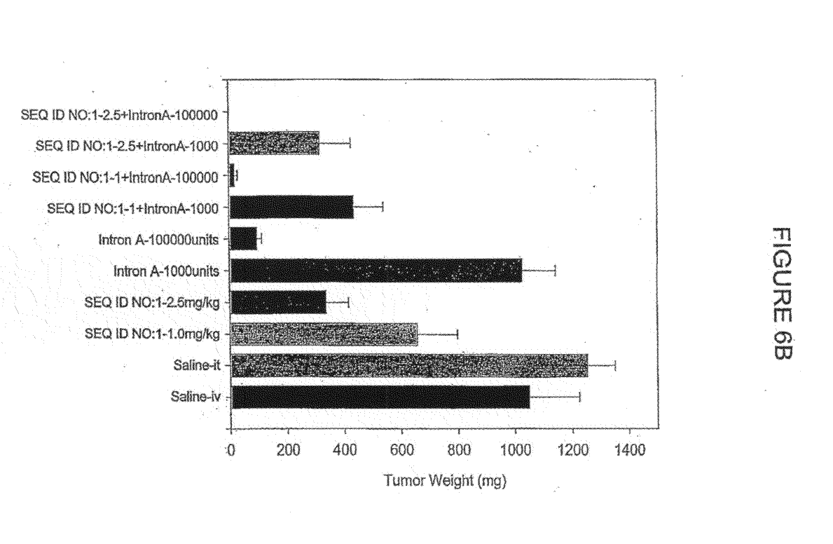

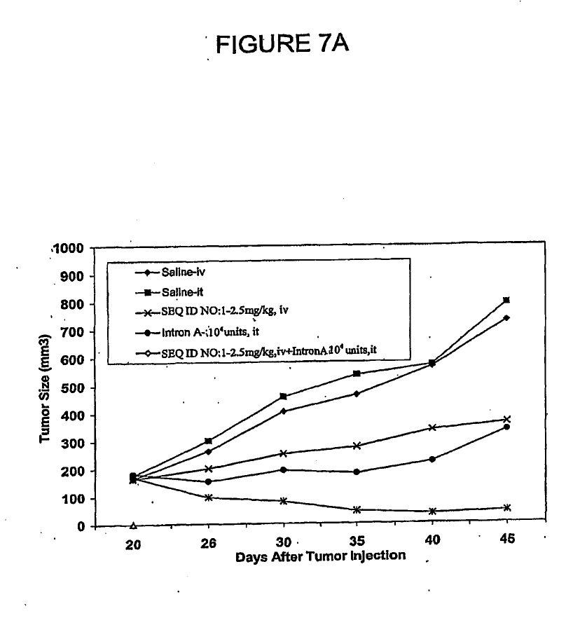

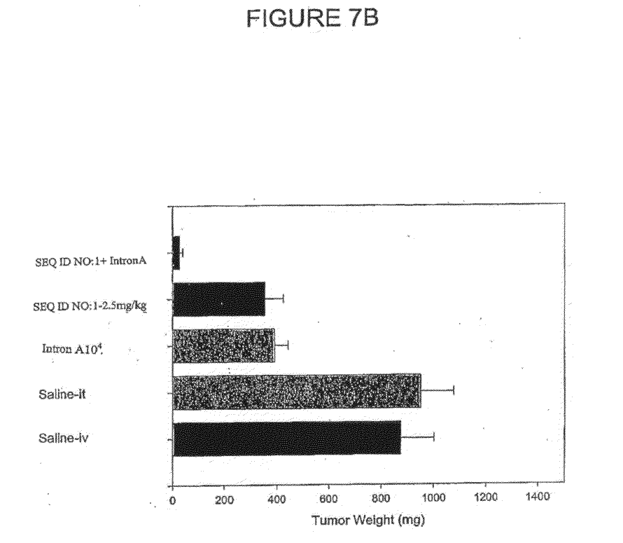

The results of the tumour volume at different time points after tumour inoculation are shown in FIG. 7A and the tumour weights at the experimental endpoint are shown in FIG. 7B, illustrating that both SEQ ID NO:1 and Intron A have significant anti-tumour efficacy as monotherapies and in combination the effect is at least additive.

### Example 8

### Alternative Models for in vivo Testing of SEQ ID NO:1 in Combination with Interferon Alpha

As SCID mice are immunocompromised, an alternative model that can be used to examine the efficacy of interferon alpha administration in combination with SEQ ID NO:1 in the treatment of renal carcinoma is the RenCa model of murine renal cancer in normal mice (for example, immune competent Balb/c mice). Although this model utilizes syngeneic RenCa cells, the target sequence for SEQ ID NO:1 in the ribonucleotide reductase R2 mRNA is conserved in mice and as such the efficacy of SEQ ID NO:1 in combination with IFN alpha can be effectively tested in the RenCa murine tumour model. Treatments and protocols such as those outlined in the Examples above can be employed.

The RenCa model of murine renal cancer can also be used to test other immunotherapeutics, including various interleukins, in combination with SEQ ID NO:1, for example, following the protocol provided in Example 10 below.

### Example 9

### Phase I/II Clinical Trials to Evaluate SEQ ID NO:1 and Interferon Alpha Combination Therapy in Patients with Advanced or Metastatic Renal Cell Carcinoma

As described in the previous Examples, mice bearing A498 renal tumour xenografts that were treated with the combination of SEQ ID NO:1 and interferon alpha demonstrated tumour regression. Partial regression and stabilization was also observed in mice bearing Caki 1 renal tumour xenografts. This response to the combination treatment was dose dependent and also sequence specific, as treatment with a control oligonucleotide did not improve the efficacy of interferon alpha. In view of the positive results obtained in these pre-clinical evaluations, clinical trials to investigate the efficacy of SEQ ID NO:1 in combination with interferon alpha in human patients can be designed and conducted. Appropriate clinical trials can be designed following general procedures known in the art and described generally herein, as well as applicable guidelines, including the guidelines regarding the Good Clinical Practices of the Title 21 Code of Federal Regulations, including proposed Part 54.

By way of example, the following outline and general criteria for a Phase I/II study are provided upon which suitable clinical trials could be based. One skilled in the art will appreciate that modifications can be made to the described protocol prior to, or during, the trial to account for such factors as availability of resources, patient dropout, the occurrence of dose-limiting toxicities, and the like.

As SEQ ID NO:1 is regarded as a cytostatic agent when used in the treatment of cancer in humans (see results of clinical trials in Example 15) rather than a cytotoxic agent, it is assumed that the combination of SEQ ID NO:1 and interferon alpha will have a greater impact on time to progression (TTP) in patients than on overt tumour shrinkage. Accordingly, an appropriate primary endpoint for a phase I/II study of the combination of SEQ ID NO:1 and interferon alpha is progression-free survival (PFS). For comparison, the Memorial Sloan-Kettering Cancer Center database of 463 first line-patients that were treated with interferon (Motzer et al. J. Clinical Oncology 2002 20(1): 289-296) can be used.

Thus, the primary objectives of a Phase I/II study for the combination of SEQ ID NO:1 and interferon alpha would be:
(1) To determine the recommended Phase II dose of SEQ ID NO:1 when given in combination with Interferon (interferon alpha)
(2) To evaluate progression-free survival (PFS) relative to historical controls in patients with advanced or metastatic renal cell carcinoma

Secondary objectives could include:
(1) To determine the objective response rate of SEQ ID NO:1 plus Interferon in patients with advanced or metastatic renal cell carcinoma
(2) To assess the duration of objective responses (Complete & Partial) and minor responses
(3) To assess the frequency and duration of stable disease
(4) To assess the toxicity of SEQ ID NO:1 in combination with Interferon.

The pharmacokinetic objective of the study would be to characterize the pharmacokinetic profile of SEQ ID NO:1 on escalating doses and at the Phase II dose.

### Study Description

The following outline relates to the evaluation of SEQ ID NO:1 in combination with Interferon as a first line therapy in the treatment of advanced or metastatic renal carcinoma.

### Eligibility Criteria

Histologically confirmed diagnosis of advanced or metastatic renal cell carcinoma
No prior treatment with cytokines, chemotherapy or other systemic therapy
Measurable or evaluable disease
Age≧18y; KPS≧70% or ECOG 0-2; Informed consent
Able to maintain central venous line access throughout study
Absence of neurological metastases or bone metastases with expected neurologic involvement
Life expectancy at least 3 months
Standard exclusions and acceptable laboratory criteria for SEQ ID NO:1 and Interferon alpha

### Trial Design

An open label, non-randomized Phase I/II study
Phase I portion will escalate the dose of SEQ ID NO:1 in combination with Interferon in order to develop the recommended dose for the Phase II portion (see below)
Dose escalation decisions will be based on dose limiting toxicities (DLTs) seen in the first cycle
A single-stage design will be utilized for the Phase II portion
It is estimated that approximately 12 patients will be required for the Phase I portion of the study and approximately 40 (36+10% due to drop outs) for the Phase II portion. Since the Phase II contingent will include 6 Phase I patients at the Phase II dose, it is estimated that 34 additional patients will need to be enrolled in the Phase II portion of the study.
Proposed duration of study is at least 56 days.

### Dose Selection

In a previous phase I single agent study of SEQ ID NO:1 in patients with solid tumours or melanoma, the maximum tolerated dose (MTD) was achieved at 222.0 mg/m²/day. No evidence of toxicity was seen in human subjects at a dose of 148.0 mg/m²/day. Accordingly, an appropriate starting dose of SEQ ID NO:1 for the study would be 148.0 mg/m²/day, with an escalation in combination therapy to 185.0 mg/m²/day (one dose level below the MTD in the previous Phase I monotherapy study) or until the MTD for SEQ ID NO:1 in combination with interferon is determined.

Interferon dose escalation schemes of 6-9 MIU subcutaneous 3 times per week have been previously used in a combination therapy study of Interferon and a cell cycle inhibitor (Dutcher J P, et al. Proceedings of ASCO Volume 22 p. 213, Abstract 854, 2003). In addition, a dose of 10 MIU subcutaneous 3 times per week has been achieved in a combination therapy study of Interferon and the immunomodulator levamisole with no significant increases in treatment toxicity (Askoy H, et. al., Int Urol Nephrol. 2001 33(3):457-9). Accordingly, an appropriate starting dose for interferon would be 6 MIU, with an escalation to 9 or 10 MIU. Exemplary dose escalations for the combination of SEQ ID NO:1 and interferon are provided in the following Table.

TABLE 4
Exemplary Phase I Dose Escalation

| | Interferon-alpha 2b | |
|---|---|---|
| Dose Level | (3x per week) | SEQ ID NO: 1 |
| 2 | 9 (3-9)^{d} MIU | 185.0 mg/m²/d |
| 1 | 6 (3-6)^{d} MIU | 185.0 mg/m²/d |
| 0* | 6 (3-6)^{d} MIU | 148.0 mg/m²/d |

*Starting dose level
^{d}Allowable individualized dosing range of interferon if dose adjustments required

### Exemplary Treatment Regimens

Treatment cycle can be of 3-weeks or 4-weeks duration
For a 21-day cycle, SEQ ID NO:1 can be administered as continuous intravenous infusion for 14 days at a starting dose of 148.0 mg/m²/day followed by 7 days of rest. For a 28-day cycle, SEQ ID NO:1 can be administered as continuous intravenous infusion for 21 days at a starting dose of 148.0 mg/m²/day followed by 7 days of rest.
Interferon can be administered subcutaneously 3 times per week during either the 21-day or 28-day cycle at a starting dose of 6 MIU
Patients can continue indefinitely unless progressive disease or intolerable toxicity develops
Patients who respond, have minor responses, or no change in disease status may continue on treatment until disease progression.

### Dose Limiting Toxicities (DLTs)

Appropriate levels of DLTs applicable to dose escalation studies can be established by the skilled clinician. Toxicities are generally classified according to the National Cancer Institute (NCI) Common Terminology Criteria for Adverse Events (CTCAE), Version 3.0, Jun. 10, 2003. Laboratory abnormalities, other than hematologic or coagulation parameters or complement levels determined by the investigating clinician to be attributable to a pre-existing medical condition or to the patient's malignancy, may be considered toxicities.

### Dose Modifications

Dose modification to account for the development of dose limiting toxicities (DLTs) in a patient may be made during the study. The following provides guidelines for appropriate dose modifications that may be made for each of the test agents.

The dose of SEQ ID NO:1 may be reduced one level or interrupted at the investigator's clinical discretion, until the DLT grade reduces. Dosage may then be resumed at the reduced dose level. In the event of unexpected toxicities, the same actions may be taken except that resumption of treatment may be at the full dose at the investigator's discretion. Doses can be adjusted to the following levels 185.0 mg/m²/d, 148.0 mg/m²/d, and 74.0 mg/m²/d.

If severe toxicities develop, the dosage of interferon can be modified, for example, by a 50% reduction, or therapy can be temporarily discontinued until the adverse reactions abate. If toxicity requires a dose to be held, that dose will be omitted and the next scheduled dose will be delivered as scheduled without delay. A reduced dose will not be re-escalated throughout the remainder of the patient's time on study. If a patient requires multiple dose reductions, the patient should be removed from the study. An appropriate maximum number of dose reductions of interferon in a patient is two. In addition, study treatment should be discontinued if immunotherapy is withheld or interrupted for 4 weeks.

### Criteria for Evaluation

An appropriate primary efficacy outcome would be the PFS rate at 6 months. For the purpose of sample size calculation, the PFS at 6 months can be based on historical control data (see, Motzer, 2002, supra). The PFS for a patient is defined as the time from start of therapy to progression.

As an alternative, the PFS rate can be compared to a control arm comprising patients treated with interferon alpha alone. If this alternative is employed then the sample size will need to be doubled.

As a secondary endpoint, objective response rate is determined using standard Response Evaluation Criteria in Solid Tumors (RECIST) Guidelines (Therasse P, et al. New guidelines to evaluate the response to treatment in solid tumours. J of the National Cancer Institute, 92(3): 205-216). The older alternative standard World Health Organization definitions (WHO Handbook for reporting results of cancer treatment. World Health Organization Offset Publication No. 48; 1979) is considered as an alternate study design option

### Subsequent Trials

Subsequent randomized Phase II(b)/III trials can be conducted which would typically involve an enrolment of approximately 400 patients. Confirmation assessment of clinical benefit may optionally be obtained from such trials by continuing to survival endpoint.

### Example 10

### In vivo Testing of SEQ ID NO:1 in Combination with Interleukin-2 in a Mouse Xenograft Model of Human Renal Carcinoma (Caki-1)

Cell line: Human renal carcinoma cell line (Caki-1) was grown as monolayer culture in Minimum essential medium (α-MEM) supplemented with 10% fetal bovine serum (FBS), 0.1 mM non-essential amino acid, 1.0 mM sodium pyruvate, 100 U/ml penicillin, 100 μg/ml streptomycin and 0.25 μg/ml amphotericin B and 2 mM L-alanyl-1-glutamine at 37° C. in an atmosphere of 5% CO₂ in air. The tumour cells were routinely subcultured twice weekly by trypsin-EDTA treatment. The cells were harvested from subconfluent logarithmically growing culture by treatment with trypsin-EDTA and counted for tumour inoculation.

Tumour Inoculation: An acclimation period of at least 7 days was allowed between animal receipt and commencement of tumour inoculation. When the female SCID mice were 6-7 weeks of age (20-25 g), each mouse was subcutaneously injected with 1×10⁷Caki-1 human renal carcinoma cells in 0.1 ml of PBS at the right flank of the mice to induce tumour growth.

Dose Preparation and Treatment: The dose for antisense oligonucleotide drug (SEQ ID NO:1) was formulated using the following procedure. From a concentrated stock solution (usually prepared in ddH₂O at a concentration of 50 mg/ml and kept at −80° C.), SEQ ID NO:1 was diluted with saline to achieve a final target concentration of 10 mg/ml on the first day of dosing. Enough SEQ ID NO:1 was diluted so that the compound could be administered by bolus infusion into the tail vein every other day for the duration of the experiment. The treatments were started 7 days after the tumour cell inoculation when the tumour size reached an approximate volume of 100 mm³. Each treatment group contained 10 tumour-bearing mice. The dose and treatment schedules for IL2 are described below. Routes of administration are as follows: intravenous (i.v.) and intraperitoneal (i.p.).

Groups and treatments: The following treatments were evaluated in this experiment:
1. Saline treated group: saline 0.1 ml/mouse/48 hours, i.v., n=10
2. SEQ ID NO:1 treated group: 10 mg/kg/48 hours in 0.1 ml saline, i.v., n=10 (17 treatments total)
3. IL2 treatment cycle: 8 days (treated 4 days then stop 2 days then treated another 4 days)
   I—High Dose (20000 unit)/2 times for one day treatment, i.p. n=10
   II—Low Dose (5000 unit)/2 times for one day treatment, i.p. n=10
4. SEQ ID NO:1+IL-2 treated group-I: n=10
5. SEQ ID NO:1+IL-2 treated group-II: n=10

Endpoints: The anti-tumour effects of SEQ ID NO:1 and IL-2, a standard immunotherapeutic for renal carcinoma, alone or in combination, on tmnour growth, was determined. Tumour sizes were measured every week day from day 7 after the tumour cell inoculation in two dimensions using a caliper, and the volume was expressed in mm³ using the formula: V=0.5 a×b², where a and b are the long and short diameters of the tumour, respectively. Mean tumour volumes calculated from each measurement were then plotted in a standard graph to compare the anti-tumour efficacy to that of controls. A day after the last treatment, tumours were excised from the animals and their weights were measured. A standard bar graph was used to demonstrate the differences in tumour weights with each bar representing mean tumour weight calculated from 10 animals.

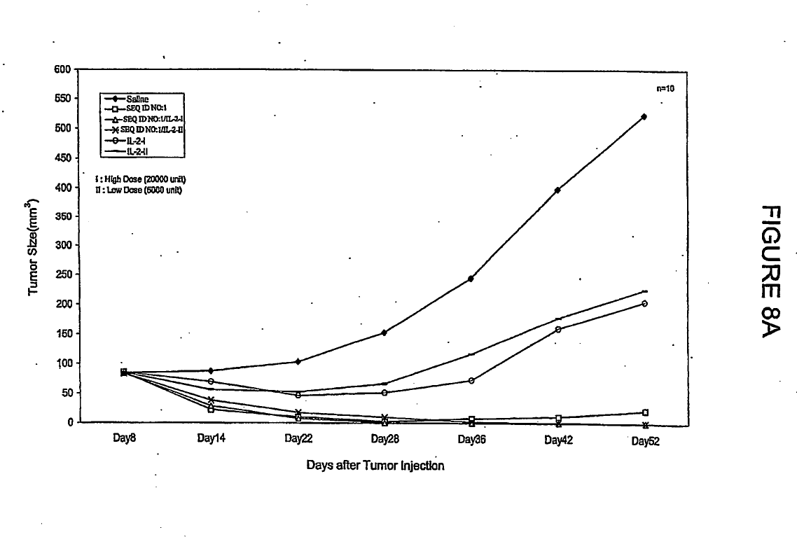

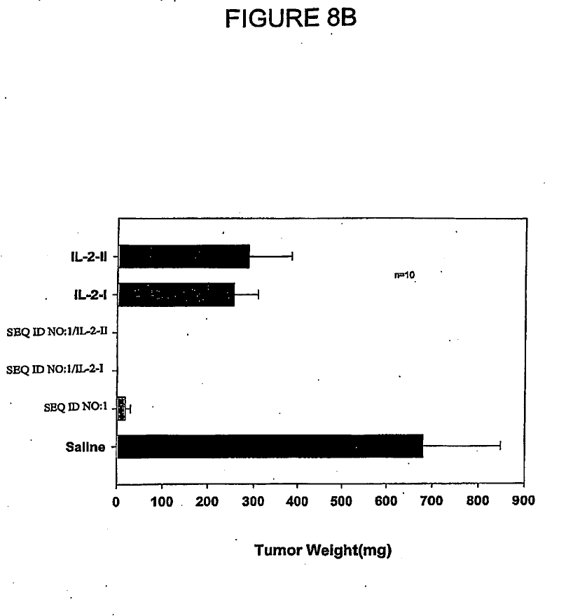

The results of the tumour volume at different time points after tumour inoculation are shown in FIG. 8A and the tumour weights at the experimental endpoint are shown in FIG. 8B, illustrating that both SEQ ID NO:1 and IL-2 have anti-tumour efficacy as monotherapies but that SEQ ID NO:1 is superior to both high and low dose IL-2 therapy. In combination the effect is at least additive.

The results from the combination treatments of SEQ ID NO:1 and IL-2 at both low and high dose combination (5000 U and 20000 U, respectively) demonstrated dramatic regression of renal cell tumours in mice. All 10 mice in each group demonstrated total regression of all tumours.

### Example 11

### Potential Clinical Trial Design to Study SEQ ID NO:1 and Interleukin-2 Combination Therapy in Patients with Advanced or Metastatic Renal Cell Carcinoma

As described in the preceding example, mice bearing Caki 1 renal tumour xenografts that were treated with the combination of SEQ ID NO:1 and II-2 demonstrated tumour regression. In view of the positive results obtained in these pre-clinical evaluations, clinical trials to investigate the efficacy of SEQ ID NO:1 in combination with IL-2 in human patients can be designed and conducted. Appropriate clinical trials can be designed following general procedures known in the art and described generally herein and applicable guidelines, including the guidelines regarding the Good Clinical Practices of the Title 21 Code of Federal Regulations, including proposed Part 54. By way of example, the following outline for a Phase I/II study is provided upon which suitable clinical trials could be based. One skilled in the art would appreciate that modifications can be made to the described parameters prior to, or during, the trial to account for such factors as availability of resources, patient dropout, the occurrence of dose-limiting toxicities, and the like.

### Trial Design

Given that IL-2 therapy is an approved therapy for renal cell carcinoma in humans, the outline provided below relates to the evaluation of the combination of SEQ ID NO:1 with interleukin-2 (IL-2) in the treatment of renal cell carcinoma. This combination can also be evaluated as first-line systemic therapy with patient eligibility criteria would be similar to that described in Example 9 above.
Phase I portion will escalate the dose of SEQ ID NO:1 in combination with IL-2 in order to develop the recommended dose for the Phase II portion (see exemplary dose escalations, in Table 5)
Dose escalation decisions will be based on dose limiting toxicities (DLTs) seen in the first cycle
Phase II population to include some Phase I patients at the Phase II dose

TABLE 5
Exemplary Phase I Dose Escalation for
SEQ ID NO: 1 in Combination with IL-2*

| Dose Level | IL-2 (every 8 hours (q8 h))* | SEQ ID NO: 1 |
|---|---|---|
| 1 | 300,000 IU/kg (0.019 mg/kg) | 148.0 mg/m²/d |
| 2 | 300,000 IU/kg (0.019 mg/kg) | 185.0 mg/m²/d |
| 3 | 600,000 IU/kg (0.037 mg/kg) | 185.0 mg/m²/d |

*Alternatively, the range of starting and patient individualized doses commonly used in clinical practice for IL-2 may be the basis for an alternative dose escalation design to determine the recommended dose level.

### Exemplary Treatment Regimen

Treatment cycle can be of 3-weeks or 4-weeks duration
For a 21-day cycle, SEQ ID NO:1 can be administered as continuous intravenous infusion for 14 days at a starig dose of 148.0 mg/m²/day followed by 7 days of rest. For a 28-day cycle, SEQ ID NO:1 can be administered as continuous intravenous infusion for 21 days at a starting dose of 148.0 mg/m²/day followed by 7 days of rest.
For a 28 day cycle, IL-2 can be administered as a 15 minute infusion every 8 hours (q8h) up to a maximum of 14 doses followed by a rest period each 14 day period during the cycle (doses will be withheld as necessary for toxicity)
After the first two cycles, an interruption of IL-2 of one cycle may be required at any time as clinically indicated
Combination with IL-2 requires daily monitoring during dose administration

### Example 12

### In Vivo Testing of SEQ ID NO:1′ in Combination with Various Chemotherapeutic Agents in Mouse Xenograft Models

SEQ ID NO:1 has demonstrated in vivo efficacy, alone and in combination with a number of standard chemotherapeutic agents, as demonstrated in this and the following Examples 13 to 15. Efficacy was demonstrated in xenograft models representing a variety of different tumour types and in clinical trials against a number of different cancers, indicating the broad applicability of treatment with SEQ ID NO:1, alone or in combination with one or more other therapeutic agent, to cancers in general.

### Example 12.1

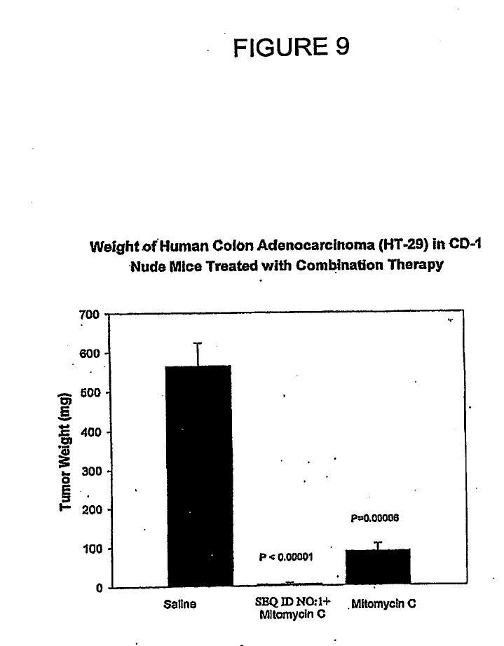

HT-29 human colon cancer cells (3×10⁶ cells in 100 μl of PBS) were subcutaneously injected into the right flank of 6-7 weeks old female CD-1 nude mice. After the size of tumour reached an approximate volume of 50 mm³, 4 days post tumour cell injection, mitomycin C was administered by bolus infusion into the tail vein at days 4, 11 and 18 with a dose of 3.5 mg/kg/week. Antitumour effect of mitomycin C was further compared to that of SEQ ID NO:1 in combination with mitomycin C. SEQ ID NO:1 was administered by bolus infusion into the tail vein every day at 6 mg/kg and mitomycin C was administered intravenously at days 4, 11 and 18 with a dose of 3.5 mg/kg/week, one hour after the treatments with SEQ ID NO:1. Control animals received saline alone for the same period as SEQ ID NO:1. All treatments were stopped at day 22. A day after the last treatment, tumours were excised from the animals and their weights were measured. A standard bar graph was used to demonstrate the differences in tumour weights with each bar representing mean tumour weight calculated from S animals. As illustrated, mitomycin C treatments resulted in significant delay of tumour growth compared to saline control. The antitumour effects elicited by the combination of SEQ ID NO:1 and mitomycin C were more potent than those obtained using mitomycin C alone (see FIG. 9).

### Example 12.2

HT-29 human colon cancer cells (3×10⁶ cells in 100 μl of PBS) were subcutaneously injected into the right flank of 5-6 week old female CD-1 nude mice. After the size of tumour reached an approximate volume of 100 mm³, 7 days post tumour cell injection, SEQ ID NO:1 was administered by bolus infusion into the tail vein every other day at 10 mg/kg. Control animals received saline alone for the same period. Antitumour effect of SEQ ID NO:1 was further compared to that of CPT-11 alone or that of SEQ ID NO:1 in combination with CPT-11. CPT-11 was administered intraperitoneally for 5 days in a row from day 7-12 with a dose of 20 mg/kg in 100 μl saline. All treatments were stopped at day 32. A day after the last treatment, tumours were excised from the animals and their weights were measured. A standard bar graph was used to demonstrate the differences in tumour weights with each bar representing mean tumour weight calculated from 9 animals. As illustrated, SEQ ID NO:1 treatments resulted in significant delay of tumour growth compared to saline control. The delay in tumour growth achieved with SEQ ID NO:1 was superior to the inhibitory effects observed with CPT-11 alone. The combination treatments of SEQ ID NO:1 and CPT-11 showed excellent cooperative effects that were more potent than either agent alone (see FIG. 10).

### Example 12.3

HT-29 human colon cancer cells (3×10⁶ cells in 100 μl of PBS) were subcutaneously injected into the right flank of 6-7 week old female CD-1 nude mice. Treatments started at day 5 post tumour cell injection, SEQ ID NO:1 and 5-FU were administered as outlined below:
Groups and treatment:
Group 1. Saline treated group: saline:0.1 ml/2 days, i.v.;
Group 2. SEQ ID NO:1 treated group: 10 mg/kg/2 days in 100 μL saline, i.v.;
Group 3. 5-FU treated group:13 mg/kg/5 days/week (one week on and one week off), i.v.;
Group 4. SEQ ID NO:1 plus 5-FU treated group.

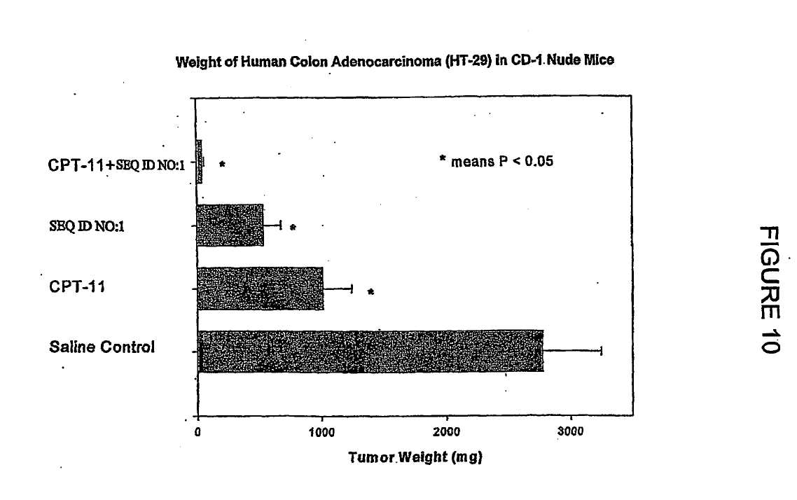

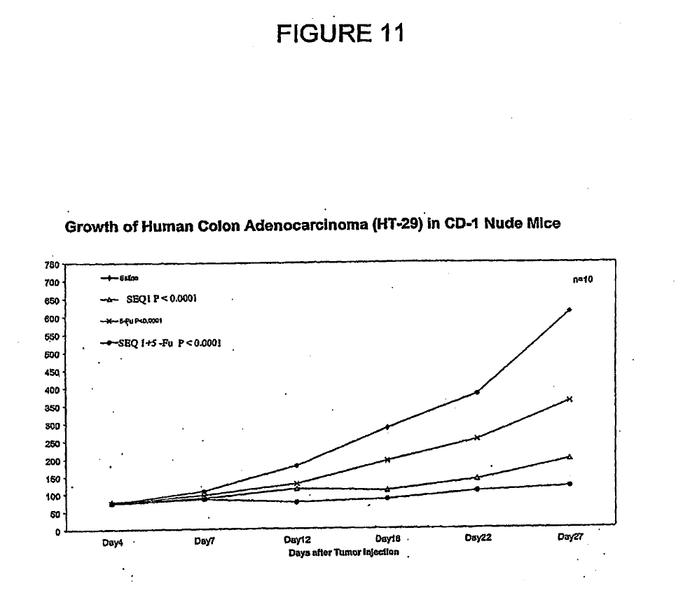

The growth of human colon tumour cells in CD-1 nude mice treated with SEQ ID NO:1 alone and in combination with 5-FU is depicted in FIG. 11.

### Example 12.4

HT-29 human colon cancer cells (3×10⁶ cells in 100 μl of PBS) were subcutaneously injected into the right flank of 5-6 week old female CD-1 nude mice. Treatments started 4 days post tumour cell injection, SEQ ID NO: 1 and Capecitabine were administered as outlined below:
Group and Treatment:
Group 1: treated with 0.2 ml vehicle solution for capecitabine, o.p.;
Group 2: treated with 0.1 ml saline, i.v.;
Group 3: treated with 359 mg/kg/day×5/w Capecitabine in 0.2 ml vehicle solution, o.p.;
Group 4: treated with 10 mg/kg/2 days SEQ ID NO:1 in 0.1 ml saline i.v.;
Group 5: treated with 10 mg/kg/2 days SEQ ID NO:1 in 0.1 ml saline, i.v. plus 359 mg/kg/day×5/w capecitabine in 0.2 ml vehicle solution, o.p.

The growth of human colon tumour cells in CD-1 nude mice treated with SEQ ID NO:1 alone and in combination with Capecitabine is depicted in FIG. 12.

### Example 2.5

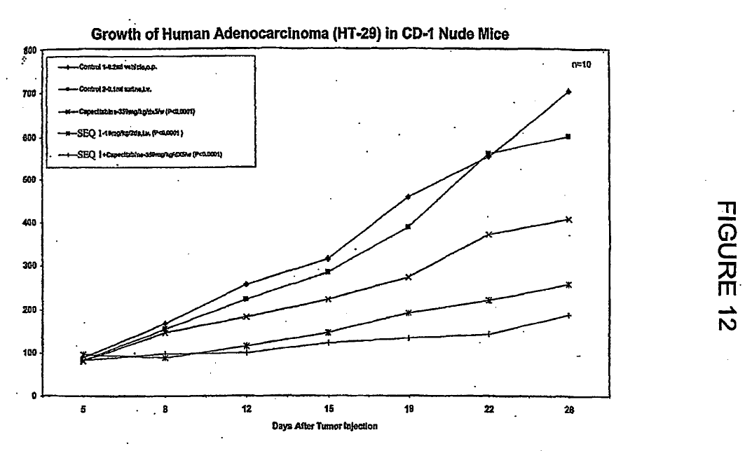

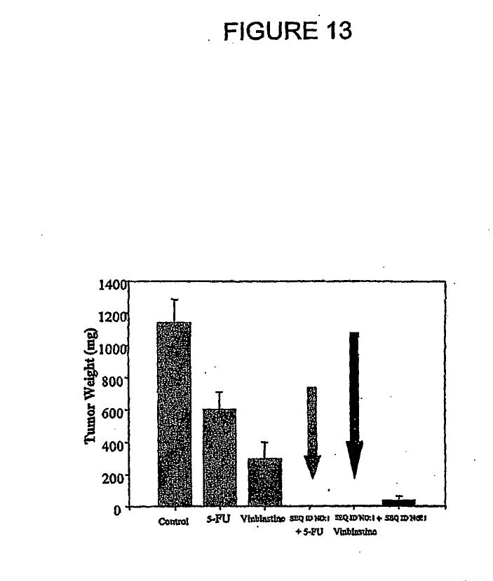

Caki-1 human renal cancer cells (1×10⁷ cells in 1001 of PBS) were subcutanteously injected into the right flank of 6-7 weeks old female SCID mice. After the size of tumour reached an approximate volume of 200 mm³, 7 days post tumour cell injection, SEQ ID NO:1 was administered by bolus infusion into the tail vein every other day at 10 mg/kg. Control animals received saline alone for the same period. The antitumour effect of SEQ ID NO:1 was further compared to that of two chemotherapeutic agents: 5-FU and vinblastine. 5-FU was administered intraperitoneally at days 7-13, 21-27 and 35-36 with a dose of 13 mg/kg/day, while vinblastin was administered intraperitoneally at days 7, 14, 21, 28 and 35 at a dose of 0.6 mg/kg/week. Antitumour effects of each of these compounds were further compared to those of SEQ ID NO:1 in combination with 5-FU or with vinblastin. The two chemotherapeutic agents were applied as described above, one hour after the treatments with SEQ ID NO:1 when combination treatments occurred on the same day. All treatments were stopped at day 36. A day after the last treatment, tumours were excised from the animals and their weights were measured. A standard bar graph was used to demonstrate the differences in tumour weights with each bar representing mean tumour weight calculated from 5 animals. As illustrated, SEQ ID NO:1 treatments resulted in significant delay of tumour growth compared to saline control. The delay in tumour growth achieved with SEQ ID NO:1 was superior to the inhibitory effects observed with each of two chemotherapeutic compounds. The combination of SEQ ID NO:1 with 5-FU or vinblastine was more effective than either agent alone (see FIG. 13).

### Example 12.6

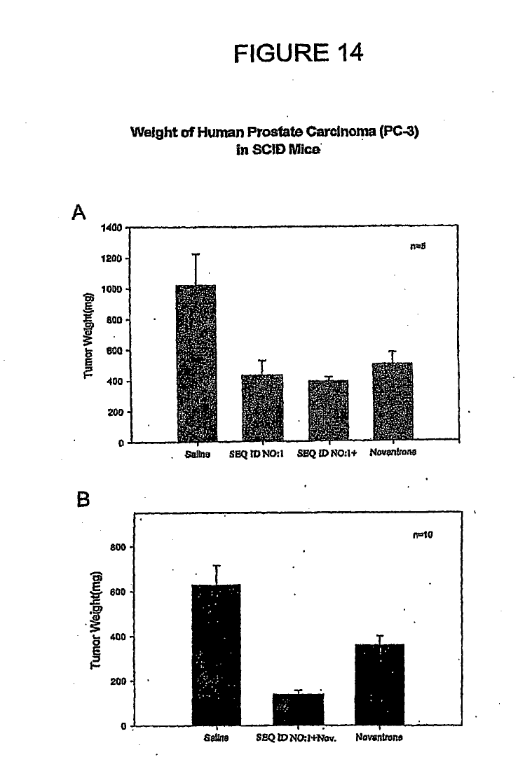

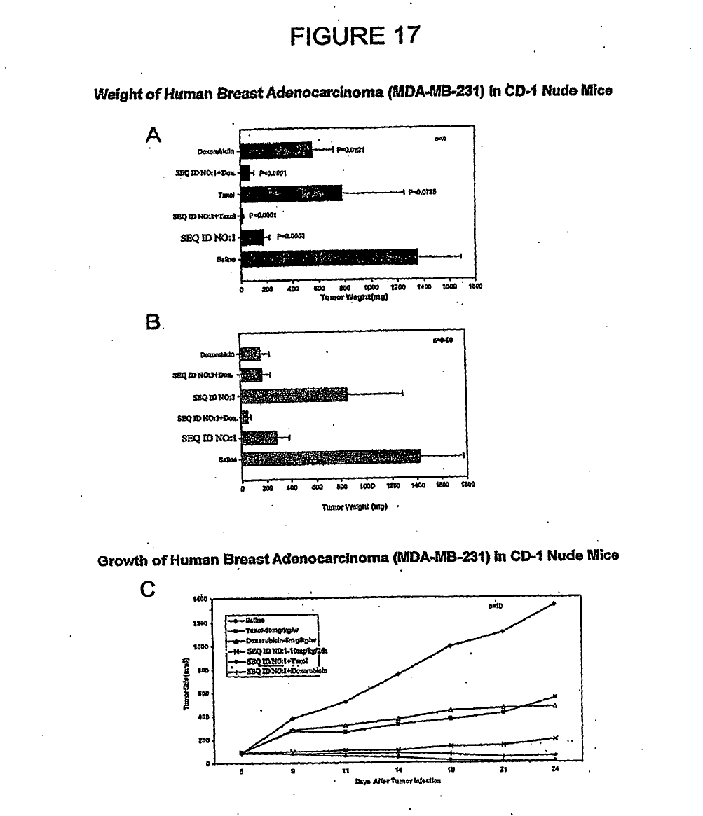

FIG. 14 shows results from two independent experiments. In both experiments, PC-3 human prostatic cancer cells (1×10⁷ cells in 100 μl of PBS) were subcutaneously injected into the right flank of 6-7 weeks old male SCID mice. After the size of tumour reached an approximate volume of 50 mm³, 14 days post tumour cell injection, SEQ ID NO:1 was administered by bolus infusion into the tail vein every other day at 10 mg/kg 18 times (FIG. 14A) or 17 times (FIG. 14B), respectively. Control animals received saline alone for the same period. Antitumour effect of SEQ ID NO:1 was further compared to that of mitoxantrone (Novantrone®) alone or in combination. Mitoxantrone was administered intravenously once at the beginning of the treatments at a dose of 2 mg/kg (FIG. 14A) or once a week for four weeks at a reduced dose of 0.8 mg/kg (FIG. 14B). All treatments were stopped at day 50 (FIG. 14A) or 48 (FIG. 14B), respectively. A day after the last treatment, tumours were excised from the animals and their weights were measured. A standard bar graph was used to demonstrate the differences in tumour weights with each bar representing mean tumour weight calculated from 5 (FIG. 14A) or 10 (FIG. 14B) animals. As illustrated in FIG. 14A, SEQ ID NO:1 treatments resulted in significant delay of tumour growth compared to saline control. The delay in tumour growth achieved with SEQ ID NO:1 was similar to the inhibitory effects observed with mitoxantrone alone. The combination of SEQ ID NO:1 with mitoxantrone (SEQ ID NO:1+) showed some additive antitumour effects. FIG. 14B shows mitoxantrone alone resulted in significant delay of tumour growth and the combination therapy was more potent than mitoxantrone monotherapy.

### Example 12.7

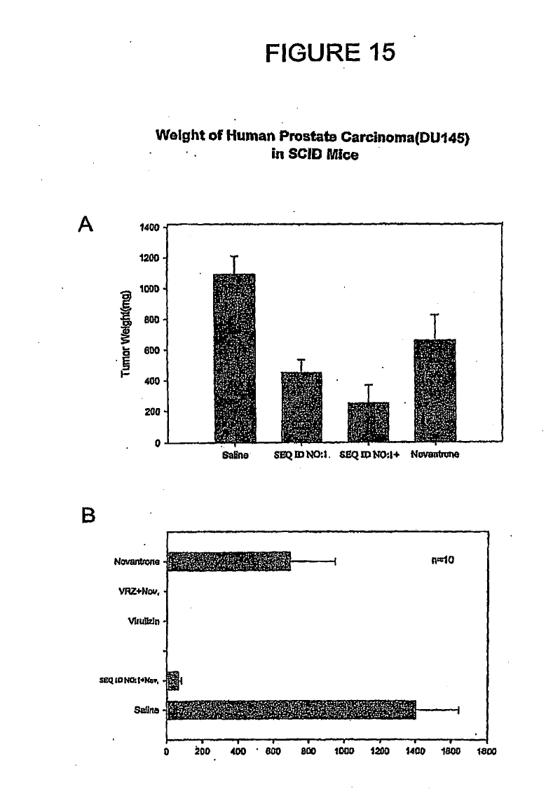

FIG. 15 shows results from two independent experiments. In both experiments, DU145 human prostatic cancer cells (1×10⁷ cells in 100 μl of PBS) were subcutaneously injected into the right flank of 6-7 weeks old male SCID mice. After the size of tumour reached an approximate volume of 50 mm³, 13 FIG. 15A) or 11 (FIG. 15B) days post tumour cell injection, SEQ ID NO:1 was administered by bolus infusion into the tail vein every other day at 10 mg/kg 15 times (FIG. 15A) or 14 times (FIG. 15B), respectively. Control animals received saline alone for the same period. Antitumour effect of SEQ ID NO:1 was further compared to that of mitoxantrone (Novantrone®) alone or in combination. Mitoxantrone was administered intravenously once at the beginning of the treatments at a dose of 2 mg/kg (FIG. 15A) or once a week for four weeks at a reduced dose of 0.8 mg/kg (FIG. 15B). All treatments were stopped at day 42 (FIG. 15A) or 38 (FIG. 15B); respectively. A day after the last treatment, tumours were excised from the animals and their weights were measured. A standard bar graph was used to demonstrate the differences in tumour weights with each bar representing mean tumour weight calculated from 0.5 (FIG. 15A) or 10 (FIG. 15B) animals. As illustrated in FIG. 15A, SEQ ID NO:1 treatments resulted in significant delay of tumour growth compared to saline control. The delay in tumour growth achieved with SEQ ID NO:1 was similar to the inhibitory effects observed with mitoxantrone alone. The combination of SEQ ID NO:1 with mitoxantrone (SEQ ID NO:1+) showed some additive antitumour effects. In FIG. 15B, mitoxantrone alone resulted in significant delay of tumour growth and the combination therapy was more potent than mitoxantrone monotherapy.

### Example 12.8

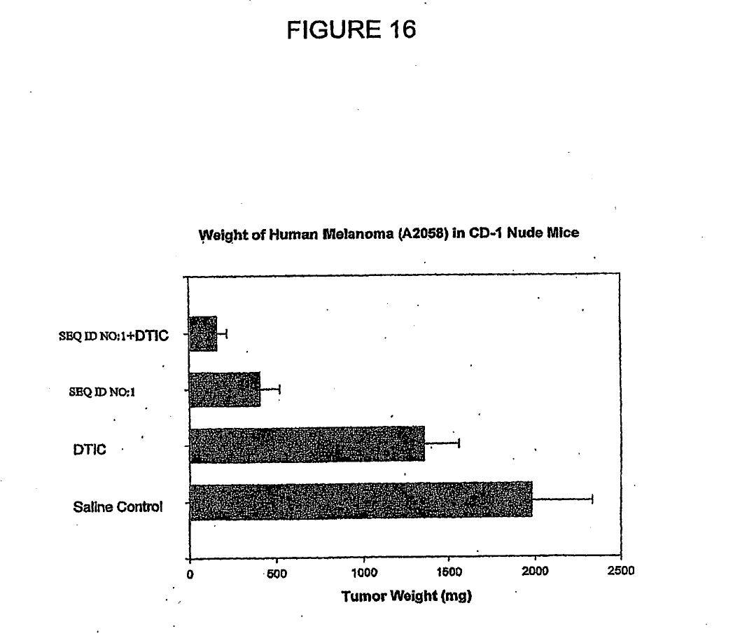

A2058 human melanoma cells 1×10⁷ cells in 100 μl of PBS) were subcutaneously injected into the right flank of 6-7 week old female CD-1 nude mice. A2058 is a metastatic melanoma cell line. After the size of tumour reached an approximate volume of 100 mm³, 6 days post tumour cell injection, SEQ ID NO:1 was administered by bolus infusion into the tail vein every other day at 10 mg/kg. Control animals received saline alone for the same period. Antitumour effect of SEQ ID NO:1 was further compared to that of dacarbazine (DTIC) alone or that of SEQ ID NO:1 in combination with DTIC. DTIC was administered intravenously for 5 days in a row from day 6-10 at a dose of 80 mg/kg in 100 μl saline. All treatments were stopped at day 24. A day after the last treatment, tumours were excised from the animals and their weights were measured. A standard bar graph was used to demonstrate the differences in tumour weights with each bar representing mean tumour weight calculated from 10 animals. As illustrated, SEQ ID NO:1 treatments resulted in significant delay of tumour growth compared to saline control. The delay in tumour growth achieved with SEQ ID NO:1 was superior to the inhibitory effects observed with DTIC alone. The combination of SEQ ID NO:1 and DTIC was more potent than either agent alone (FIG. 16).

### Example 12.9

FIG. 17 shows results from three independent experiments. MDA-MB-231 human breast cancer cells (1×10⁷ cells in 100 μl of PBS) were subcutaneously injected into the right flank of 6-7 weeks old female CD-1 nude mice. After the size of tumour reached an approximate volume of 100 mm³, 5 days post tumour cell injection, SEQ ID NO:1, or the scrambled control oligonucleotide (SEQ ID NO:3) were administered by bolus infusion into the tail vein every other day at 10 mg/kg. Control animals received saline alone for the same period. Antitumour effect of SEQ ID NO:1 was further compared to that of taxol or doxorubicin alone or in combination. Taxol was administered intravenously once a week at a dose of 10 mg/kg for three (FIG. 17A) or four weeks (FIG. 17C). Doxorubicin was administered intravenously once a week at a dose of 5 mg/kg for first three weeks (FIG. 17A) or for two weeks (FIG. 17C). All treatments were stopped at day 33 (FIG. 17A) or at day 26 (FIG. 17C), respectively. A day after the last treatment, tumours were excised from the animals and their weights were measured. A standard bar graph was used to demonstrate the differences in tumour weights with each bar representing mean tumour weight calculated from 10 animals (FIGS. 17A & 17B). In FIG. 17C, antitumour activities were estimated by the inhibition of tumour volume, which was measured with calipers. Each point represents mean tumour volume calculated from 10 animals per experimental group. As illustrated, SEQ ID NO:1 treatments resulted in a delay of tumour growth compared to saline control in all three experiments. The delay in tumour growth achieved with SEQ ID NO:1 was superior to the inhibitory effects observed with taxol or doxorubicin alone. The combination therapy of SEQ ID NO:1 with taxol or doxorubicin was more potent than either monotherapy. FIG. 17B demonstrates that a control oligonucleotide that has the same base composition as SEQ ID NO:1, but is not complementary to R2 mRNA has no significant anti-tumour activity as a monotherapy and does not cooperate with doxorubicin, suggesting that the effects of SEQ ID NO:1 are sequence specific.

### Example 12.10

SK-OV-3 human ovary adenocarcinoma cells (1×10⁷ cells in 100 μl of PBS) were subcutaneously injected into the right flank of 6-7 weeks old female CD-1 nude mice. After the size of tumour reached an approximate volume of 100 mm³, 6 days post tumour cell injection, SEQ ID NO:1 was administered by bolus infusion into the tail vein every other day at 10 mg/kg 17 times. Control animals received saline alone for the same period. Antitumour effect of SEQ ID NO: 1 was further compared to that of taxol or cisplatin alone or in combination. Taxol was administered intravenously once a week for first three weeks and intraperitoneally once a week for next two weeks at a dose of 10 mg/kg. Cisplatin was administered intravenously once a week for first three weeks and intraperitoneally once a week for next two weeks at a dose of 4 mg/kg. All treatments were stopped at day 40. Antitumour activities were estimated by the inhibition of tumour volume, which was measured with caliper. Each point represents mean tumour volume calculated from 9 animals per experimental group. As illustrated, SEQ ID NO:1 treatments resulted in significant delay of tumour growth compared to saline control. The delay in tumour growth achieved with SEQ ID NO:1 was similar or superior to the inhibitory effects observed with taxol or cisplatin alone, respectively. The combination therapy of SEQ ID NO:1 with taxol or cisplatin was more potent than either monotherapy (FIG. 18).

Results of SEQ ID NO; 1 treatment in combination with various chemotherapeutic agents are summarized in Table 6.

TABLE 6
Summary of SEQ ID NO: 1 Treatment in Combination with
Standard Chemotherapy Drugs

| | | | Tumour weight | |
|---|---|---|---|---|
| | | | as % of | |
| Tumour | Mouse | Treatment | saline control | |
| Caki (renal) | CD-1 | SEQ ID NO: 1 | 3.3 | |
| | | 5-FU | 52 | |
| | | Vinblastine | 26 | |
| | | SEQ ID NO: 1 + 5-FU | 0 | |
| | | SEQ ID NO: 1 + Vinblastine | 0 | |
| HT-29 | SCID | Mitomycin C | 15 | |
| (colon) | | SEQ ID NO: 1 + Mitomycin C | 0.8 | |
| HT-29 | CD-1 | SEQ ID NO: 1 | 19 | |
| (colon) | | CPT-11 | 36 | |
| | | SEQ ID NO: 1 + CPT-11 | 1.4 | |
| MDA- | CD-1 | SEQ ID NO: 1 | 12.6 | |
| MB-231 | | Taxol | 58 | |
| (breast) | | Doxorubicin | 41 | |
| | | SEQ ID NO: 1 + Taxol | 1 | |
| | | SEQ ID NO: 1 + Doxorubicin | 4.8 | |
| A2058 | CD-1 | SEQ ID NO: 1 | 20 | |
| (melanoma) | | DTIC | 68 | |
| | | SEQ ID NO: 1 + DTIC | 8 | |
| PC-3 | SCID | Novantrone | 57 | |
| (prostatic) | | SEQ ID NO: 1 + Novantrone | 21 | |
| DU145 | SCID | SEQ ID NO: 1 | n.a. | 41 |
| (prostatic) | | | | |
| | | Novantrone | 40 | 60 |
| | | SEQ ID NO: 1 + Novantrone | 4.6 | 23 |

| | | | |
|---|---|---|---|
| **SK-OV-3 | CD-1 | SEQ ID NO: 1 | 42 |
| (ovary) | | Taxol | 49 |
| | | Cisplatin | 67 |
| | | SEQ ID NO: 1 + Taxol | 24 |
| | | SEQ ID NO: 1 + Cisplatin | 27 |

Results shown are mean tumour weights presented as a percentage of saline treated controls.
**is tumour volume data as percentage of saline control.

### Example 13

### In vivo Testing of SEQ ID NO:1 Alone or in Combination with Various Chemotherapeutic Agents in Drug-Resistant Tumours

### Example 13.1

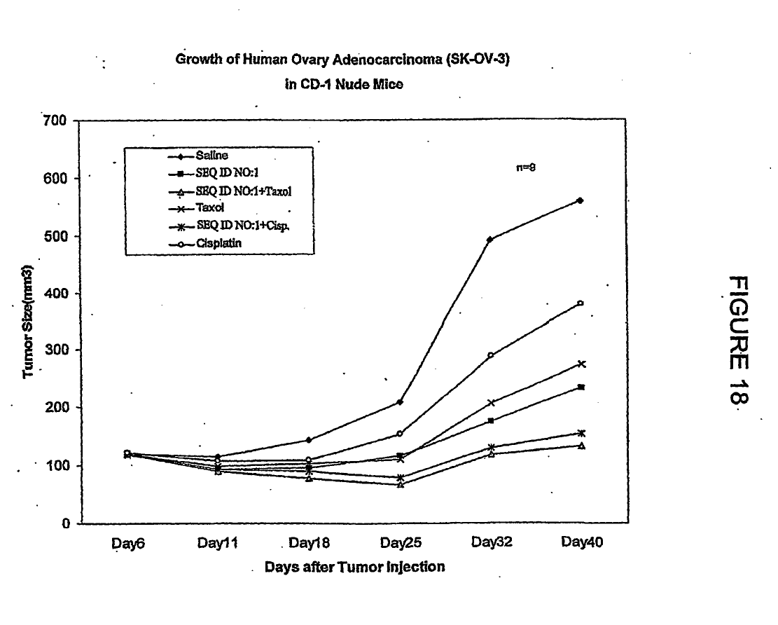

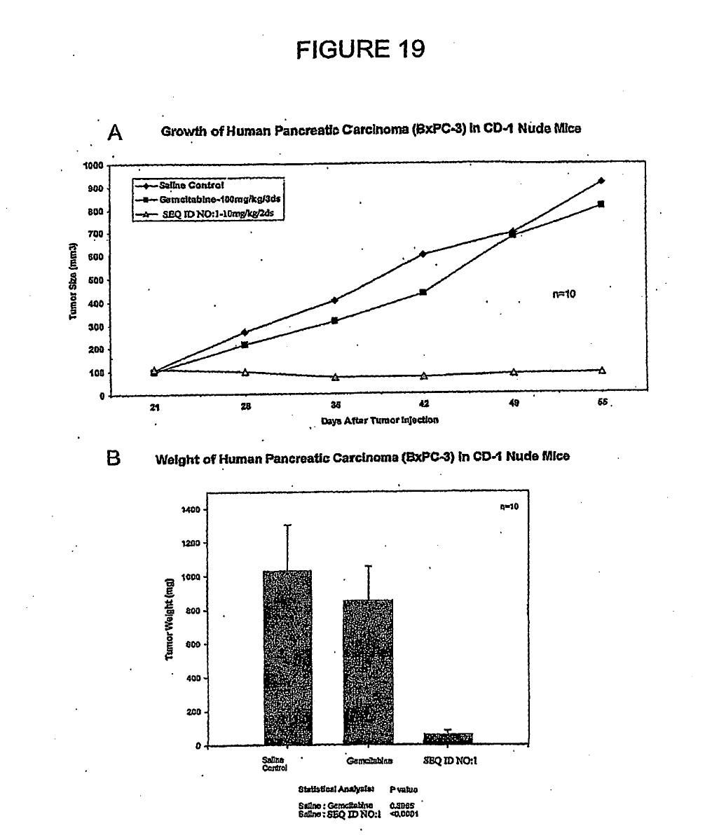

BxPC-3 human pancreatic carcinoma cells (3×10⁶ cells in 100 μl of PBS) were subcutaneously injected into the right flank of 6-7 weeks old female CD-1 nude mice. BxPC-3 is a gemcitabine resistant call line. After the size of tumour reached an approximate volume of 100 mm³, 21 days post tumour cell injection, SEQ ID NO:1 was administered by bolus infusion into the tail vein every other day at 10 mg/kg 17 times. Control animals received saline alone for the same period. Antitumour effect of SEQ ID NO:1 was further compared to that of Gemcitabine. Gemcitabine was administered intravenously every three days at a dose of 100 mg/kg. Antitumour activities were estimated by the inhibition of tumour volume, which was measured with caliper. Each point represents mean tumour volume calculated from 10 animals per experimental group. As illustrated, SEQ ID NO: 1 treatments resulted in significant delay of tumour growth compared to saline control. As expected, treatment with Gemcitabine during the same period was ineffective against Gemcitabine-resistant tumour (FIGS. 19A & B).

### Example 13.2

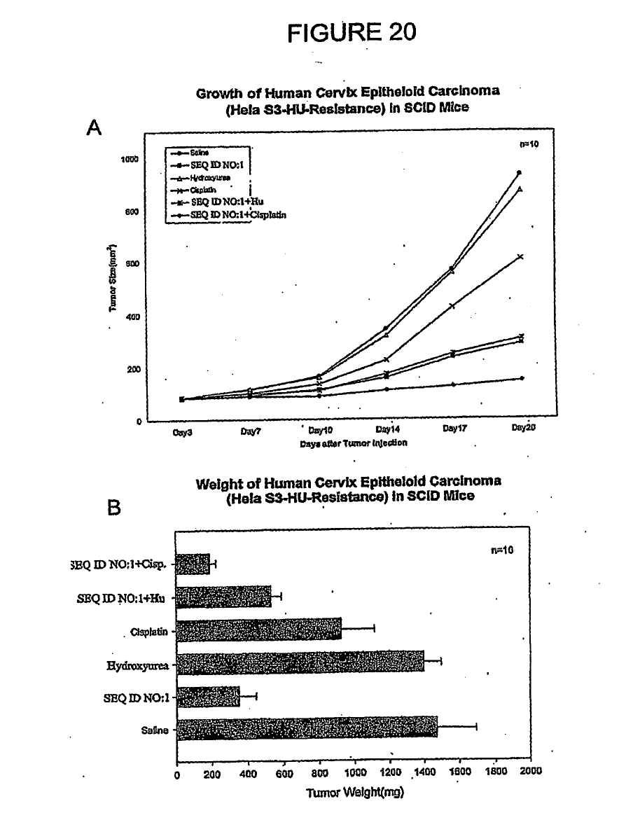

Hela S3 human cervix epitheloid carcinoma cells (5×10⁵ cells in 100 μl of PBS) were subcutaneously injected into the right flank of 6-7 weeks old female SCID mice. Hela S3 is a hydroxyurea resistant cell line. After the size of tumour reached an approximate volume of 100 mm³, 3 days post tumour cell injection, SEQ ID NO:1 was administered by bolus infusion into the tail vein every other day at 10 mg/kg 6 times. Control animals received saline alone for the same period. Antitumour effect of SEQ ID NO:1 was further compared to that of Hydroxyurea or Cisplatin alone or in combination. Hydroxyurea was administered intraperitoneally every day at a dose of 250 mg/kg for 10 days. Cisplatin was administered intravenously once a week for three weeks at a dose of 4 mg/kg. Antitumour activities were estimated by the inhibition of tumour volume, which was measured with caliper. Each point represents mean tumour volume calculated from 10 animals per experimental group. As illustrated, SEQ ID NO:1 treatments resulted in significant delay of tumour growth compared to saline control. As expected, treatment with Hydroxyurea during the same period was ineffective against Hydroxyurea-resistant tumour. The delay in tumour growth achieved with SEQ ID NO:1 was superior to the inhibitory effects observed with Cisplatin alone, which was used as a positive control. The combination therapy of SEQ ID NO:1 with Hydroxyurea was only as effective as SEQ ID NO:1 monotherapy, as expected. The combination therapy of SEQ ID NO:1 with Cisplatin, however, was more potent than either monotherapy (FIGS. 20A & B).

### Example 13.3

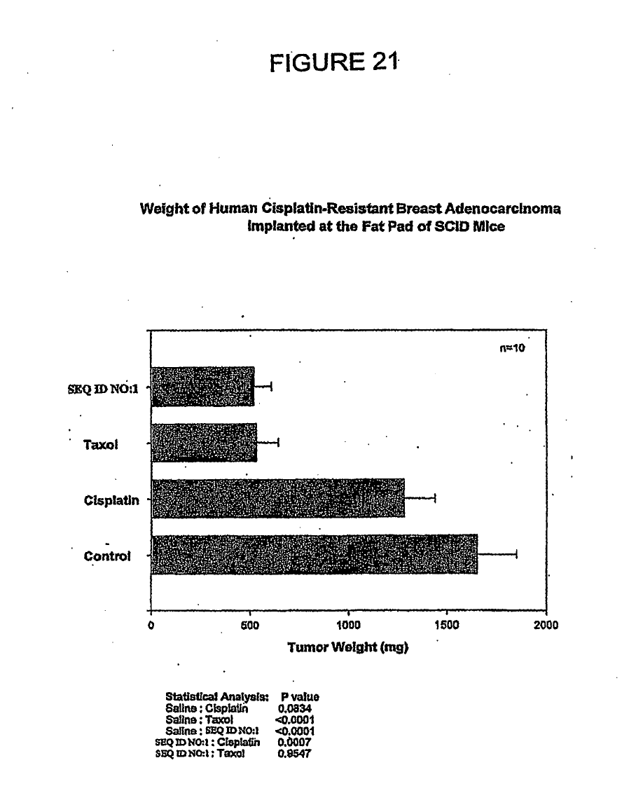

MDA-CDDP-S4 human in vivo-selected Cisplatin-resistant breast adenocarcinoma cells (4×10⁶ cells in 100 μl of PBS) were injected into the fat pad (inside of right leg) of 6-7 weeks old female SCID mice. After the size of tumour reached an approximate volume of 100 mm³, 7 days post tumour cell injection, SEQ ID NO:1 was administered by bolus infusion into the tail vein every other day at 10 mg/kg 9 times. Control animals received saline alone for the same period. Antitumour effect of SEQ ID NO: 1 was further compared to that of Cisplatin or Taxol alone. Cisplatin was adminstered intravenously once a week for three weeks at a dose of 4 mg/kg. Taxol was administered intravenously once a week for three weeks at a dose of 10 mg/kg. Antitumour activities were estimated by the inhibition of tumour volume, which was measured with caliper. Each point represents mean tumour volume calculated from 10 animals per experimental group. As illustrated, SEQ ID NO: 1 treatments caused significant reduction of tumour weight compared to saline control. As expected, treatment with Cisplatin during the same period was ineffective against Cisplatin-resistant tumour. The delay in tumour growth achieved with SEQ ID NO: 1 was similar to the inhibitory effects observed with Taxol, which was used as a positive control FIG. 21).

### Example 13.4

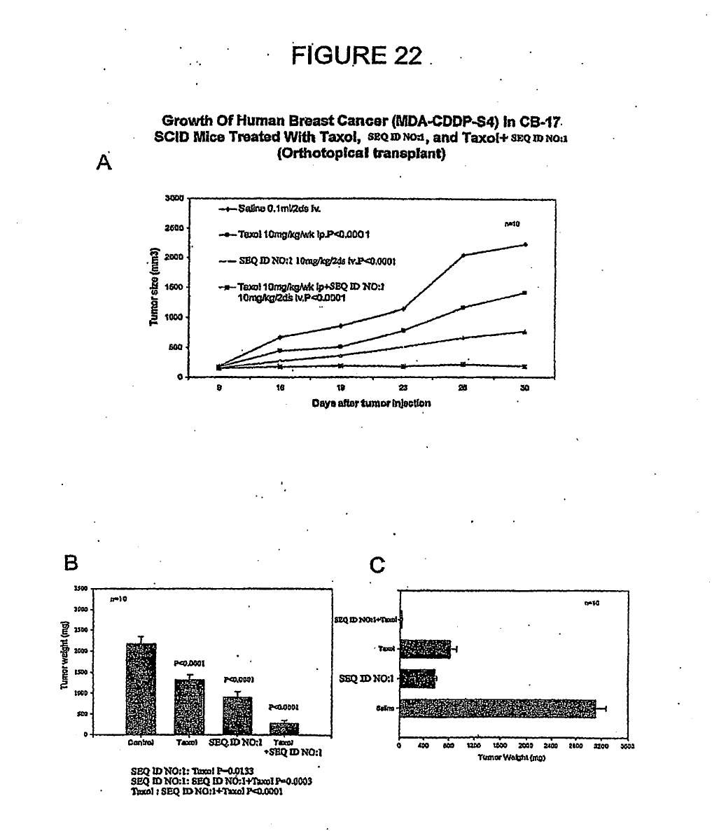

MDA-CDDP-S4 human in vivo-selected Cisplatin-resistant breast adenocarcinoma cells (4×10⁶ cells in 100 μl of PBS) were injected into the fat pad (inside of right leg) of 6-7 weeks old female CB-17 SCID mice. After the size of tumour reached an approximate volume of 100 mm³, 9 days post tumour cell injection, SEQ ID NO:1 was administered by bolus infusion into the tail vein every other day at 10 mg/kg. Control animals received saline alone for the same period. Antitumour effect of SEQ ID NO:1 was further compared to that of Taxol alone and in combination. Taxol was administered i.p. once a week at a dose of 10 mg/kg. Antitumour activities were estimated by the inhibition of tumour volume FIG. 22A), which was measured with calipers. Each point represents mean tumour volume calculated from 10 animals per experimental group. Animals were sacrificed and tumour weights taken at the end of the study (FIG. 22C). SEQ ID NO:1 treatments caused significant reduction of tumour weight compared to saline control. The delay in tumour growth achieved with SEQ ID NO:1 was superior to the inhibitory effects observed with Taxol, which was used as a positive control. The effects of combined treatment were greater than either treatment alone. This study was repeated with similar results (FIG. 22B).

### Example 13.5

MDA-MB435-To.1 human Taxol-resistant breast adenocarcinoma cells (4×10⁶ cells in 1001 of PBS) were injected into the fat pad (inside of right leg) of 6-7 weeks old female SCID mice. After the size of tumour reached an approximate volume of 100 mm³, 20 days post tumour cell injection, SEQ ID NO:1 was administered by bolus infusion into the tail vein every other day at 10 mg/kg 15 times. Control animals received saline alone for the same period. Antitumour effect of SEQ ID NO:1 was further compared to that of Cisplatin or Taxol alone. Cisplatin was administered intravenously once a week for four weeks at a dose of 4 mg/kg. Taxol was administered intravenously once a week for four weeks at a dose of 20 mg/kg.

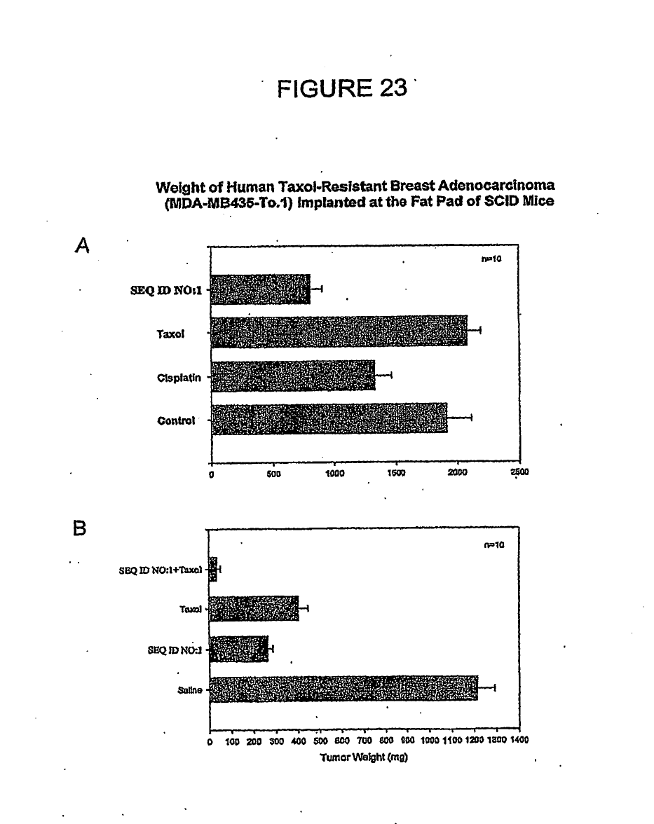

Antitumour activities were estimated by the inhibition of tumour volume, which was measured with caliper. Bach point represents mean tumour volume calculated from 9-10 animals per experimental group. As illustrated, SEQ ID NO:1 treatments caused significant reduction of tumour weight compared to saline control. As expected, treatment with Taxol during the same period was ineffective against Taxol-resistant tumour. The delay in tumour growth achieved with SEQ ID NO:1 was superior to the inhibitory effects observed with Cisplatin, which was used as a positive control (see FIGS. 23A & B).

### Example 13.6

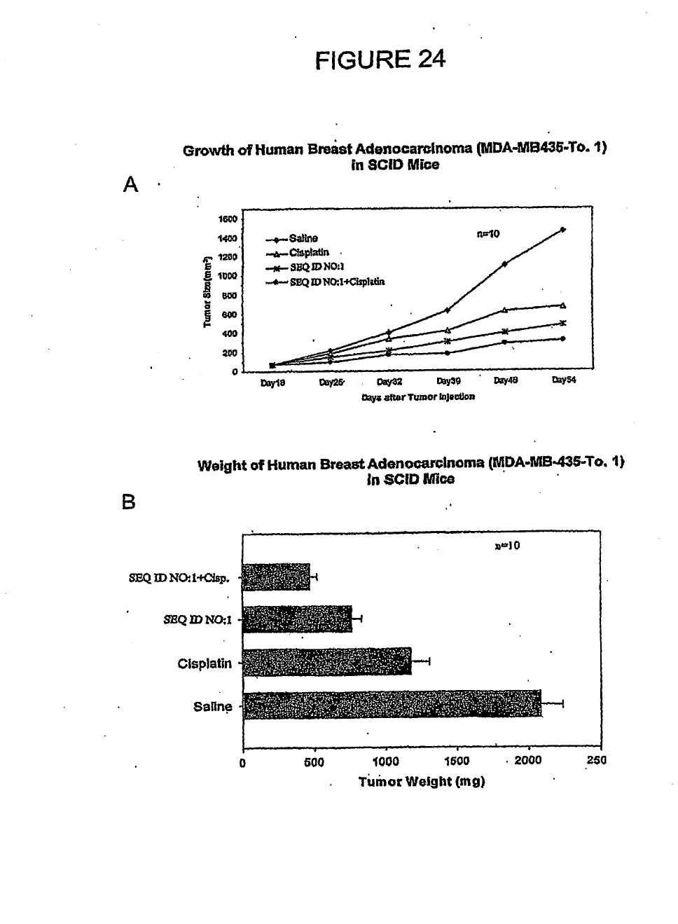

MDA-MB435-To.1 human Taxol-resistant breast adenocarcinoma cells (4×10⁶ cells in 100 μl of PBS) were injected into the fat pad (inside of right leg) of 6-7 weeks old female CB-17 SCID mice. After the size of tumour reached an approximate volume of 100 mm³, 17 days post tumour cell injection, SEQ ID NO:1 was administered by bolus infusion into the tail vein every other day at 10 mg/kg. Control animals received saline alone for the same period. Antitumour effect of SEQ ID NO:1 was compared to that of Cisplatin alone and in combination. Cisplatin was administered intravenously once a week for four weeks at a dose of 4 mg/kg. Antitumour activities were estimated by the inhibition of tumour volume, which was measured with caliper. Each point represents mean tumour volume calculated from 10 animals per experimental group. At the end of the study the animals were sacrificed and tumours weighed. As illustrated, SEQ ID NO:1 treatment caused significant reduction of tumour weight compared to saline control. The delay in tumour growth achieved with SEQ ID NO:1 was superior to the inhibitory effects observed with Cisplatin, which was used as a positive control. The combination of the two compounds produced anti-tumour efficacy that was superior to either one alone (see FIGS. 24A & B).

### Example 13.7

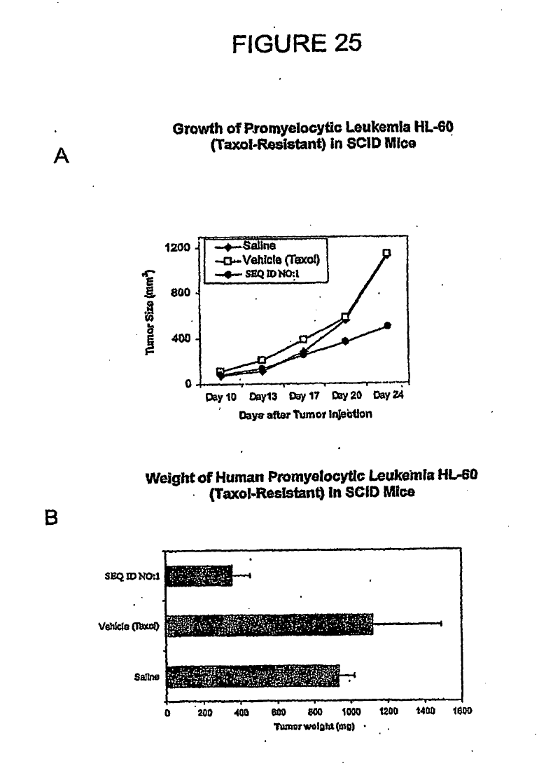

Human taxol-resistant promyelocytic leukaemia cells (HL-60) (7×10⁶ cells in 100 μl of PBS) were injected into the right flank of 6-7 weeks old female SCID mice. After the size of tumour reached an approximate volume of 100 mm³, 10 days post tumour cell injection, SEQ ID NO:1 was administered by bolus infusion into the tail vein every other day at 10 mg/kg. Control animals received saline alone for the same period. The anti-tumour effect of SEQ ID NO:1 was further compared to that of taxol. Taxol was administered i.p. once a week at a dose of 10 mg/kg. Anti-tumour activity was estimated by the inhibition of tumour volume, which was measured with caliper. Each point represents mean tumour volume calculated from 10 animals per experimental group. In addition animals were sacrificed and tumour weights taken at the end of the study. SEQ ID NO: 1 treatments caused significant reduction of tumour weight compared to saline control. As expected, treatment with taxol had no effect on tumour growth or weight (see FIGS. 25A & B).

### Example 13.8

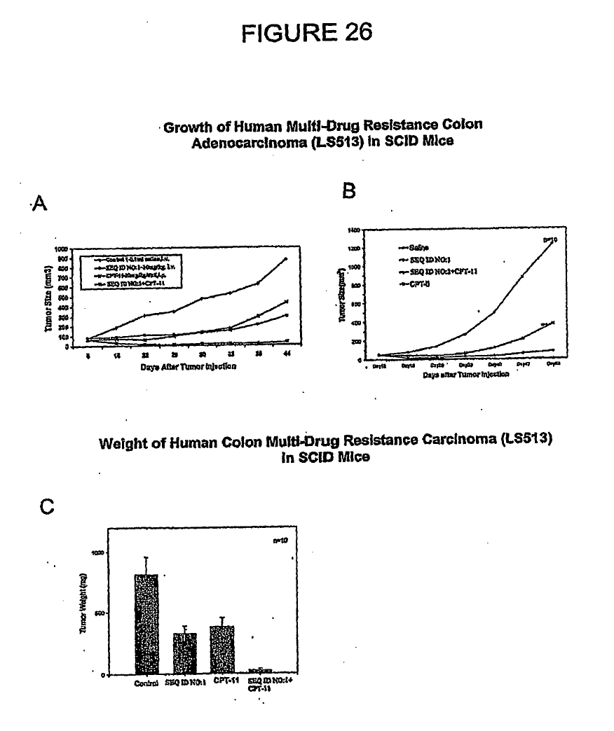

LSS13 multi-drug resistant colon carcinoma cells (1×10⁷ cells in 100 μl of PBS) were subcutaneously injected into the right flank of 6-7 weeks old female SCID mice. After the size of tumour reached an approximate volume of 100 mm⁸, 8 days post tumour cell injection, SEQ ID NO:1 was administered by bolus infusion into the tail vein every other day at 10 mg/kg. Control animals received saline alone for the same period. Antitumour effect of SEQ ID NO:1 was further compared to that of CPT-11 alone or in combination. CPT-11 was administered i.p. for 5 days at a dose of 20 mg/kg/day. Antitumour activities were estimated by the inhibition of tumour volume, which was measured with caliper. Each point represents mean tumour volume calculated from 10 animals per experimental group. Tumour weights were measured after animals were sacrificed at the end of the treatment. These cells are not resistant to CPT-11 which was used as a positive control. As illustrated, SEQ ID NO:1 treatment resulted in significant delay of tumour growth compared to saline control. SEQ ID NO:1 is as effective as CPT-11 and in combination the efficacy was greater than either treatment alone (see FIGS. 26A, B & C).

Results of SEQ ID NO:1 treatment of drug-resistant tumours alone or in combination with various chemotherapeutics are summarized in Table 7.

TABLE 7
Summary of SEQ ID NO: 1 Treatment of Drug Resistant Tumours

| | | | Tumour | |
|---|---|---|---|---|
| | | | weight as % | |
| Tumour | | | of saline | |
| Resistance | Mouse | Treatment | control | |
| LS513 (colon) | SCID | CPT-11 | 47 | |
| multi-drug resistant | | SEQ ID NO: 1 | 49 | |
| (CPT-11 sensitive) | | SEQ ID NO: 1 + CPT-11 | 3 | |
| MDA-CDDP-S4 | SCID | SEQ ID NO: 1 | 32 | |
| (breast) | | Taxol | 32 | |
| Cisplatin | | Cisplatin | 78 | |
| MDA-CDDP-S4 | SCID | SEQ ID NO: 1 | 18 | 41 |
| (breast) | | Taxol | 26 | 61 |
| Cisplatin | | SEQ ID NO: 1 + Taxol | 1 | 12 |

| | | | |
|---|---|---|---|
| MDA-MB435-To.1 | SCID | SEQ ID NO: 1 | 42 |
| (breast) | | Taxol | 109 |
| Taxol | | Cisplatin | 69 |
| MDA-MB435-To.1 | CB-17/ | SEQ ID NO: 1 | 37 |
| (breast) | SCID | Cisplatin | 56 |
| Taxol | | SEQ ID NO: 1 + cisplatin | 22 |
| HL-60 (leukemia) | SCID | SEQ ID NO: 1 | 38 |
| Taxol | | Taxol | 119 |
| BxPC-3 (pancreatic) | CD-1 | SEQ ID NO: 1 | 5.8 |
| Gemcitabine | | Gemcitabine | 83 |
| Hela S3 (cervix) | SCID | SEQ ID NO: 1 | 24 |
| hydroxyurea | | Hydroxyurea (HU) | 92 |
| | | Cisplatin | 63 |
| | | SEQ ID NO: 1 + HU | 36 |
| | | SEQ ID NO: 1 + Cisplatin | 13 |

Results shown are mean tumour weights presented as a percentage of saline treated controls.

### Example 14

### Efficacy of SEQ ID NO:1 Alone In vivo in Mouse Xenograft Models

It has been demonstrated previously using various mouse models of solid tumours, haematologic neoplasms and metastasis that treatment with SEQ ID NO:1 alone is effective in inhibiting the growth and metastasis of various tumour types (i.e. prolonging survival of mice with lymphoma or erythroleukemia). The results are summarized in Table 8. As SEQ ID NO:1 alone has demonstrated efficacy against these human cancer cell lines in vivo, it is contemplated that a combination comprising SEQ ID NO:1 with one or more immunotherapeutic agents may be equally, or more effective, in the treatment of these human tumours.

TABLE 8
Summary of Effects of SEQ ID NO: 1 on Tumour Growth and
Metastasis

| | Tumour Placed in | |
|---|---|---|
| Assay | Mouse | Results |
| Tumour | Mouse | Inhibition of tumour volume in C3H |
| Growth | Fibrosarcoma¹ | mice by approximately 80% on Day 15 |
| | | after tumour implantation |
| | | Inhibition of tumour weight by |
| | | approximately 80% |
| | | Dose-dependent decrease in tumour |
| | | weight in C3H mice at doses of 0.5 to |
| | | 30 mg/kg |
| | Human Colon | Inhibition of tumour size in CD-1 nude |
| | Adenocarcinoma | mice by approximately 80% on Day 18 |
| | | after tumour implantation |
| | | Inhibition of tumour weight by |
| | | approximately 80% |
| | | Dose-dependent decrease in tumour |
| | | weight in CD-1 nude mice at doses of |
| | | 1.0 to 6.0 mg/kg |
| | Human Melanoma | Inhibition of tumour size by |
| | | approximately 80 to 85% on Days 31 |
| | | after tumour implantation |
| | | Inhibition of tumour weight of |
| | | approximately 80% |
| | Human Breast | Inhibition of tumour weight by |
| | Adenocarcinoma | approximately 80% on Day 31 after |
| | | tumour implantation |
| | | Inhibition of tumour size by |
| | | approximately 80% |
| | Human Pancreatic | Complete inhibition of tumour growth |
| | Adenocarcinoma | up to 39 days after tumour implantation |
| | | Inhibition of tumour weight by |
| | | approximately 65% |
| | Human Ovary | Inhibition of tumour size by |
| | Adenocarcinoma | approximately 35% in Balb/c Nu-Nu |
| | | mice on Days 19 to 25 after tumour |
| | | implantationInhibition of tumour weight |
| | | of approximately 50% |
| | Human Lung | Inhibition of tumour size by |
| | Carcinoma | approximately 85% in CD-1 Nude mice |
| | | on Days 14 to 19 after tumour |
| | | implantation |
| | | Inhibition of tumour weight of |
| | | approximately 70% |
| | Human Liver | Inhibition of tumour size by 45% in |
| | Carcinoma | CD-1 nude mice on Day 30 after |
| | | tumour implantation |
| | | Inhibition of tumour weight of |
| | | approximately 65% |
| | Human | Inhibition of tumour weight of |
| | Glioblastoma- | approximately 65% |
| | Astrocytoma | |
| | Human Renal | Approximately 90% inhibition of |
| | Carcinoma | tumour size |
| | Human Renal | Approximately 97% inhibition of |
| | Carcinoma² | tumour size |
| | Human Renal | Complete regression of all tumours in |
| | Carcinoma² | treated mice |
| | Human Cervical | Inhibition of tumour size by |
| | Carcinoma² | approximately 90% in SCID mice on |
| | | Day 22 after tumour implantation |
| | | Inhibition of tumour weight of |
| | | approximately 90% |
| | Human Cervical | Inhibition of tumour size and weight by |
| | Carcinoma² | approximately 60% in SCID mice on |
| | | Day 17 after tumour implantation |
| Metastasis | Mouse | Decrease by approximately 65% in the |
| | Fibrosarcoma (ex | number of tumour metastases to lungs |
| | vivo) | |
| | Human Melanoma | Decrease by approximately 95% in the |
| | (ex vivo and in | number of tumour metastases to lungs |
| | vivo) | |
| Prolonged | Burkitt's | All untreated mice died by day 23 |
| Survival | Lymphoma | Treated mice survived beyond day 73 |
| | | with the exception of one mouse that |
| | | died at day 69 |
| | | Treatment prolonged survival |
| | Burkitt's | All untreated mice died by day 20 |
| | Lymphoma | All treated mice survived to the end of |
| | | the experimental period (140 days) |
| | | Treatment prolonged survival |
| | Erythroleukemia | All untreated mice died within 36 days |
| | | Treated mice survived beyond day 71 |
| | | except for one mouse which died at day |
| | | 22 |
| | | Treatment prolonged survival |

### Example 15

### Phase I/II Clinical Trials for SEQ ID NO:1 in Combination with Various Chemotherapeutic Agents

Examples of ongoing clinical trials and other clinical trials that have been approved by the NCl using SEQ ID NO:1 are outlined below. Details of the protocols involved for each of trials 1-7 are provided in Table 9. The following describes the Protocols involved for each trial:

### 1. Protocol LO1-1409 (Renal Cell Carcinoma)

Study Description: SEQ ID NO:1 and capecitabine combination therapy in patients with advanced or metastatic renal cell carcinoma (Phase I/II)

Population: Advanced or metastatic renal cell carcinoma having failed standard therapy

| | |
|---|---|
| Study regimen: | SEQ ID NO: 1 (CIV infusion) + |
| | capecitabine |
| | cycles: 21 days + 7 days rest |
| Status: | Ongoing in Phase II |

Dosing: SEQ ID NO:1 was administered as a continuous intravenous infusion for 21 days at a starting dose of 148.0 mg/m²/day in combination with capecitabine administered orally at a fixed dose of 1660 mg/m²/day (divided into two daily doses for 21 days) followed by 7 days of rest.

### 2. Protocol L6093 (Breast)

Study Description: A Phase II Study of SEQ ID NO:1 and Capecitabine in the treatment of Metastatic Breast Cancer

Population: Breast cancer, metastatic and failing 2 or more prior regimens

| | |
|---|---|
| Study regimen: | SEQ ID NO: 1 + Capecitabine |
| | 14 days in 21 day cycle |
| Subjects: | 40 (2 Stages: 20 ea) |
| Status: | Ongoing study |

### 3. Protocol L6104 (NSCLC)

Study Description: A Phase I/II Trial of SEQ ID NO: 1 and Docetaxel in Metastatic or Advanced Non-Small Cell Lung Cancer

Population: Metastatic or unresectable locally advanced NSCLC

| | |
|---|---|
| Study regimen | SEQ ID NO: 1 + Docetaxel |
| Subjects: | 42 (12 Phase I; 30 Phase II) |
| Status: | Ongoing study |

### 4. Protocol L6090 (Solid Tumours)

Study Description: A Phase I Study of SEQ ID NO:1 and Gemcitabine in Patients with Solid Tumours

Population: Solid tumours metastatic or unresectable and for which curative or palliative measures do not exist or are no longer effective.

| | |
|---|---|
| Study regimen | SEQ ID NO: 1 + Gemcitabine |
| Subjects: | 34 |
| Status: | Ongoing study |

### 5. Protocol L6108 (AML)

Study Description: A Phase I Trial of SEQ ID NO:1 in combination with high-dose cytarabine in refractory or relapsed acute myeloid leukaemia (AML)

Population: Acute myeloid leukaemia refractory or relapsed.

| | |
|---|---|
| Study regimen | SEQ ID NO: 1 + cytarabine |
| Subjects: | 30 |
| Status: | Ongoing study |

### 6. Protocol L6099 (Colorectal)

Study Description: A Phase I Trial of SEQ ID NO:1, Oxaliplatin and Capecitabine in Refractory Unresectable Colorectal Cancer

Population: Locally advanced or metastatic colorectal cancer (refractory, unresectable). Patients must have had at least one standard prior chemotherapy with no prior oxaliplatin-containing regimen.

| | |
|---|---|
| Study regimen | SEQ ID NO: 1 + oxaliplatin & capecitabine |
| Subjects: | 15-20 |
| Status: | Ongoing study |

### 7. Protocol L6102 (Prostate)

Study Description: A Phase II Study of SEQ ID NO:1 in combination with Docetaxel and Prednisone in Patients with Hormone-Refractory Prostate Cancer

Population: Patients with hormone-refractory prostate cancer and rising PSA levels (PSA≧20). ECOG 0-2, with adequate organ function

| | |
|---|---|
| Study regimen | SEQ ID NO: 1 + Docetaxel + Prednisone |
| Subjects: | 40 |
| Status: | Ongoing study |

TABLE 9
Current Clinical Trials using Antisense Oligonucleotide SEQ ID NO: 1
in Combination with Various Chemotherapeutic Agents

| Protocol | Objectives | Drug Regimen |
|---|---|---|
| 1. (1409) | To determine the recommended | SEQ ID NO: 1 + Capecitabine |
| | Phase II dose | SEQ ID NO: 1 was administered |
| | To evaluate the response rate | as a continuous intravenous |
| | To evaluate the toxicity | infusion for 21 days at a starting |
| | To determine pharmacokinetic data | dose of 148.0 mg/m²/day (phase |
| | | I) or 185 mg/m²/day (phase II) |
| | | followed by 7 days of rest |
| | | Capecitabine was administered |
| | | orally at a fixed dose of |
| | | 1660 mg/m²/day (divided into |
| | | two daily doses for 21 days) |
| | | followed by 7 days of rest. |
| 2. (L6093) | To evaluate the response rate | SEQ ID NO: 1 (148-185 mg/m2/ |
| | and response duration | day) + Capecitabine |
| | To evaluate the toxicity | (600-1000 mg/m2 bid for 14 |
| | To determine pharmacokinetic | days). |
| | data | 21 day treatment cycle. |
| | To investigate potential markers | SEQ ID NO: 1 will be |
| | of RNR inhibition and | administered as a 14-day |
| | fluoropyrimidine metabolism. | continuous IV infusion on days |
| | | 1-15 with a starting dose of 74 mg/m2/ |
| | | day. |
| | | The starting dose of capecitabine |
| | | will be 600 mg/m2 orally bid on |
| | | days 2-14. |
| | | Patients will have one week rest |
| | | and then on completion of the 21 |
| | | day cycle, start day one of next |
| | | cycle. |
| 3. (L6104) | To determine the recommended | SEQ ID NO: 1 (2-5 mg/kg/day) + |
| | Phase II dose | Docetaxel (60-75 mg/m2 IV) |
| | To assess the objective tumour | SEQ ID NO: 1 continuous IV |
| | response rate | infusion day 1 to 14 every 21 |
| | To assess the toxicity, stable | days. |
| | disease rate, time to disease | Docetaxel IV day 3 in cycle 1, |
| | progression, objective response | day 1 subsequent cycles, every |
| | duration and duration of stable | 21 days. |
| | disease. | Dosage of SEQ ID NO: 1 can be |
| | To investigate PK parameters | started at 3-5 mg/kg/day and be |
| | To measure the baseline and | reduced to 2 mg/kg/day if a dose |
| | post-treatment levels of RNR | reduction is indicated. |
| | activity | |
| 4. (L6090) | Primary | SEQ ID NO: 1 (100-185 mg/m2/ |
| | To determine the toxicity | day) + Gemcitabine |
| | profile and MTD | (400-1000 mg/m2) |
| | Secondary | In Cycle 1, the SEQ ID NO: 1 |
| | To examine PK and PD | CIV is given from day 2-16 |
| | To determine the effects on | every 28 days. Only from cycle |
| | RNR R2 subunit mRNA and | 2 onwards, SEQ ID NO: 1 CIV is |
| | protein expression | given from day 1-15 every 28 |
| | To examine the effects on | days. |
| | apoptotic markers and cell cycle | In Cycle 1, gemcitabine is given |
| | regulatory proteins and to | weekly on days 1, 8, and 15 |
| | analyze the serum biomarkers | every 28 days. Only from Cycle |
| | | 2 onwards, gemcitabine is given |
| | | weekly on days 2, 9, and 16 |
| | | every 28 days. |
| 5. (L6108) | To determine the MTD | Cohort 1 |
| | To document therapeutic | SEQ ID NO: 1 (3.5-11 mg/kg/ |
| | responses | day) + Cytarabine (2000-3000 mg/m2 |
| | To evaluate PK | q12 hours) |
| | To measure R2 mRNA | SEQ ID NO: 1 will be |
| | To assess apoptosis in leukemic | administered by continuous IV |
| | cells | infusion for a total of 168 days |
| | To measure RNR enzymatic | (days 1 to 7). |
| | activity | Cytarabine will be administered |
| | | IV over 2 hours every 12 hours |
| | | for a total of 12 doses (day 2 to |
| | | 7) |
| | | Cohort 2 |
| | | SEQ ID NO: 1 (3.5-5 mg/kg/day) + |
| | | Cytarabine (1500-2000 mg/m2 |
| | | q12 hours) |
| | | SEQ ID NO: 1 will be |
| | | administered by continuous IV |
| | | infusion for a total of 144 hours |
| | | (days 1 to 6). |
| | | Cytarabine will be administered |
| | | IV over 4 hours every day for 5 |
| | | days (days 2 to 6) for a total of 5 |
| | | doses. |
| 6. (L6099) | Primary | SEQ ID NO: 1 (2-5 mg/kg/day) + |
| | To establish maximum tolerated | Oxaliplatin (130 mg/m2) & |
| | dose | Capecitabine (600-1200 mg/m2/ |
| | To describe the toxicities at | BID) |
| | each dose level studied. | The dose of oxaliplatin will be |
| | Secondary | fixed at 75-130 mg/m2 and |
| | To evaluate the | administered IV over 2 hours on |
| | pharmacokinetics of the | day 2 of a 21 day treatment |
| | combination therapies. | cycles. After the first cycle, |
| | To evaluate levels of | oxaliplatin will be given on day |
| | ribonucleotide reductase-M2 | 1. |
| | subunit (RR-M2) mRNA levels. | The starting dose of capecitabine |
| | To quantify changes in dCTIP | will be 600 mg/m2 twice orally |
| | levels in peripheral | and will be given beginning on |
| | mononuclear cells as surrogate | day 2 of the first cycle for 28 |
| | marker of RR inhibition. | doses (14 days) and subsequently |
| | | on day 1 after initiation of SEQ |
| | | ID NO: 1. |
| | | SEQ ID NO: 1 will be given as a |
| | | continuous infusion through a |
| | | central line over 14 days |
| | | beginning on day 1 of treatment. |
| 7. (L6102) | Primary | SEQ ID NO: 1 (5 mg/kg/day) + |
| | To establish the efficacy using | Docetaxel (75 mg/m2) |
| | PSA-response rate | SEQ ID NO: 1; Continuous IV |
| | Secondary | infusion for 14 days of a 21-day |
| | To estimate objective tumour | cycle to start with a docetaxel |
| | response | bolus. |
| | To estimate the median time to | Docetaxel: Administered IV |
| | progression | every 21 days. For cycle 1 only |
| | To investigate safety and | administered on day 3. |
| | tolerability | Administered on day 1 for |
| | | subsequent cycles. |
| | To estimate the median of | Prednisone: 5 mg po bid |
| | duration of PSA-response | continuously |
| | To measure baseline and post- | |
| | treatment levels of RNR activity | |

Interim data for protocol 1409 showed that amongst the 25 response-evaluable patients at the phase II dose; 13 (52%) had stable disease (SD) as best response (median duration: 4 months, range 2-10), and 1 durable (8 months) partial response (PR) was observed. At the phase II dose, the patient with PR experienced a unidimensional tumour reduction of 39%, and the patient with the longest duration SD had a 23% tumour reduction. One additional patient at dose level 0 also had SD and a 13% decrease in tumour size. The combination of SEQ ID NO:1 and capecitabine is tolerated at the recommended phase II dose with expected toxicities. Treatment has been well tolerated with few treatment-related toxicities other than those already known to occur with these drugs with acceptable frequency.

Other clinical trials for SEQ ID NO:1 in combination with another therapeutic agent for the treatment of various cancers have been proposed in which the following dosages for SEQ ID NO:1 have been contemplated:

TABLE 10
Examples of Other Contemplated Dose Ranges for SEQ ID NO: 1

| Cancer | Dose Range for SEQ ID NO: 1 |
|---|---|
| AML | CIV day 1-21 120-280 mg/m²/day |
| AML | CIV day 1-8 3.5-11 mg/kg/day |
| AML, CML | CIV day 1-5 2-10 mg/kg/day |
| Breast | CIV day 1-21 74-185 mg/m²/day |
| | 28-day cycle |
| CML | CIV day 1-21 120-280 mg/m²/day |
| Colon | CIV day 1-21 85-185 mg/m²/day |
| Colorectal | CIV day 1-21 185 mg/m²/day |
| | 28-day cycle |
| Colorectal | CIV day 1-21 74-185 mg/m²/day |
| | 28-day cycle |
| Genitourinary | CIV day 1-21 100-185 mg/m²/day |
| Metastatic cancer | CIV day 1-14 125-185 mg/m²/day |
| | Cycles repeat every 21 days |
| NSCLC & other | CIV day 1-22 1-5 mg/kg/day |
| solid tumours | 28-day cycle |
| NSCLC | CIV day 1-14 74-185 mg/m²/day |
| | 21-day cycle |
| NSCLC | CIV day 1-21 74-185 mg/m²/day |
| | 28-day cycle |
| Pancreas | CIV day 1-21 85-185 mg/m²/day |
| | Cycles repeat every 21 days |
| | Schedule modified if significant toxicity in |
| | 2/3 patients @ dose level 1 to: |
| | CIV day 1-14 85-185 mg/m²/day |
| | Cycles repeat every 21 days |
| Pancreatic | CIV day 1-14 104-185 mg/m²/day |
| adenocarcinoma | 21-day cycle |
| Prostate | CIV day 1-21 3-5 mg/kg/day |
| | 28-day cycle |
| Prostate cancer | CIV day 1-14 111-185 mg/m²/day |
| | Cycles repeat every 21 days to 10 cycles or |
| | PD |
| Renal cell | CIV day 1-21 111-185 mg/m²/day |
| carcinoma | 28-day cycle |
| SCLC | CIV day 1-21 3-5 mg/kg/day |
| Solid tumours | CIV day 1-21 100-185 mg/m²/day |
| | 28-day cycle |
| Solid tumours | CIV day 1-21 50-185 mg/m²/day |
| | 28-day cycle |
| Solid tumours | CIV day 1-21 148-185 mg/m²/day |
| | 28-day cycle |
| Solid tumours | CIV day 1-21 90-190, RP2D mg/m²/day |
| | 28-day cycle |
| Solid tumours | CIV day 2-21 cycle 1 100-185 mg/m² and |
| | CIV day 1-21 100-185 mg/m² for |
| | subsequent cycles |
| | 28-day cycle |

CIV: Continuous intravenous infusion

The demonstrated efficacy of SEQ ID NO:1 in the wide range of situations described above indicates that it has potential application as part of a combination therapy with one or more immunotherapeutic agents in the treatment of a variety of cancers amenable to immunotherapy.

### Example 16

### Antisense Oligonucleotides against Ribonucleotide Reductase R2 Inhibit the Proliferation of Human Tumour Cells In Vitro #1

Colony forming efficiency was determined using standard protocols. Briefly, the cells were cultured for 24 hours at 37° C. in growth medium with 10% fetal bovine serum. The cells were washed in 5 ml phosphate buffered saline, pH 7.2, once prior to lipofectin +/−oligonucleotide treatment.

The test oligonucleotides were added to cell cultures in the presence of 2.5 μg of DOTMA/DOPE (Lipofectin; Life Technologies, Inc.) for four hours. The oligonucleotide was tested at 0.2 μM unless otherwise indicated. Controls were the cultures treated with lipofectin but without the oligonucleotide. After 4 hours the medium containing the oligonucleotide was removed and washed with 5 ml of growth medium. The cells were then cultured in growth medium containing 10% fetal bovine serum for seven to ten days. Surviving cells were visualized by methylene blue staining, and colonies were scored. In some experiments cell aliquots were removed from the culture and viability was determined using the trypan blue exclusion test. Results were analyzed as percent of surviving cells compared to control cells.

Two phosphorothioate antisense sequences of 20-mer, designated AS-II-336-20 and AS-II-2229B-20, directed against the R2 mRNA were made and investigated. AS-II-336-20, has the sequence 5′-TCC TGG AAG ATC CTC CTC GC-3′ (SEQ ID NO: 103), and targets the R2 message of human ribonucleotide reductase at nucleotides 336-355, based on the numbering of R2 nucleotides as shown in Table 1. AS-II-2229B-20 has the sequence: 5′-TCC CAC ATA TGA GAA AAC TC-3′ (SEQ ID NO:104), and targets the R2 message at nucleotides 2229-2248.

AS-II-336-20 was tested for the ability to inhibit the proliferation of human tumour cells (Hela). Hela S3 cells (American Type Culture Collection, Rockville, Md., ATCC), and a Hela cell line (Hela 1 mM) previously selected for resistance to the antitumour agent, hydroxyurea, were tested (see results in Table 11). Two experiments were undertaken with Hela S3 cells. With a 4 hour treatment of 0.2 μM antisense construct AS-II-336-20, inhibition of 92% and 82% was seen in colony forming efficiency in two experiments, respectively. The same experiment was repeated with the Hela 1 mM cell line and with varying concentrations of the antisense construct AS-II-336-20 (see results in Table 11) with similar results, 0.2 M was an effective concentration for inhibiting colony formation.

These data show that AS-II-336-20 is a very effective inhibitor of human tumour cell colony forming ability, and it is effective both in inhibiting the proliferation of human tumour cell colony forming ability and in inhibiting the proliferation of human tumour cells that exhibit resistance to another chemotherapeutic compound. Similarly, as shown in Table 11, AS-II-336-20 is an effective antitumour compound in experiments performed with the mouse tumour cell line, SC2, which is a highly hydroxyurea resistant mouse L cell line.

The antisense sequence AS-II-2229B-20 was also tested for the ability to inhibit the proliferation of human Hela tumour cells in relative colony forming efficiency experiments with results similar to that of AS-II-336-20 as shown in Table 11. These data show that AS-1′-2229B-20 is a potent antitumour agent when tested with Hela S3 cells and with the drug resistant Hela 1 mM cell line.

TABLE 11
Reduced colony Forming Efficiency following Treatment with R2
Antisense Oligonucleotides

| | | |
|---|---|---|
| | Conc. | |
| % Inhibition | AS-II-2229B-20 | % Inhibition |

Cell Line: HeLa
S3

| | | | |
|---|---|---|---|
| Conc. | | | |
| AS-II 336-20 | | | |
| 0 | — | 0 | — |
| 0.2 μM | 92% | 0.05 μM | 50% |
| 0.2 μM | 82% | 0.10 μM | 80% |
| | | 0.20 μM | 95% |
| | | 0.20 μM | 97% |

Cell Line: HeLa
1 mM

| | | | |
|---|---|---|---|
| 0 μM | — | 0 μM | — |
| 0.01 μM | .15% | 0.01 μM | 0 |
| 0.05 μM | 25% | 0.02 μM | 0 |
| 0.10 μM | 60% | 0.03 μM | 21% |
| 0.20 μM | 85% | 0.04 μM | 34% |
| | | 0.05 μM | 48% |
| | | 0.05 μM | 50% |
| | | 0.10 μM | 78% |
| | | 0.20 μM | 97% |
| | | 0.20 μM | 90% |

Cell Line: Mouse
SC2

| | |
|---|---|
| Conc. AS-II-326- | |
| 20 | |
| 0 | — |
| 0.2 μM | 95% |

AS-II-2229B-20 was also tested for the ability to inhibit the proliferation of the human breast cancer cell line MDA435 and found to be very effective (see Table 12).

TABLE 12
Treatment of the human breast cancer cell line MDA435 with AS-II-
2229B-20

| | Colony forming |
|---|---|
| Conc. (μM) | Inhibition |
| 0.02 | 25% |
| 0.03 | 56% |
| 0.05 | 78% |
| 0.10 | 94% |
| 0.20 | 99% |

AS-II-2229B-20 was further tested for tumour cell cytotoxicity by comparing the results obtained from treatment of human tumour and non-tumour cell populations. Hela S3 tumour cells and WI 38 normal non-tumorigenic human cells were used. Tumour cells were found to be much more sensitive to the cytotoxic effects of AS-II-2229B-20 than normal non-tumorigenic cells. For example, analysis of cells three days after antisense exposure indicated that tumour cells were approximately 5-times more sensitive to the cytotoxic effects of AS-II-2229B-20 than normal non-tumorigenic cells averaged over 4-8 determinations.

### Example 17

### Antisense Oligonucleotides against Ribonucleotide Reductase R2 Inhibit the Proliferation of Human Tumour Cells in vitro #2

An analysis of the R2 mRNA using a computer program (OLIGO, Primer Analysis Software, Version 3.4), was carried out to determine antisense sequence melting temperature, free energy properties, and to estimate potential self-dimer formation and self-complimentary properties, of a series of additional antisense sequences were designed to target the R2 message. These sequences (SEQ ID NOS: 1, and 4-102) are shown in Table 1.

To test the antisense effects of many of these sequences as phosphorthioate deoxyribonucleotides, they were examined in relative colony forming experiments conducted as described in Example 16 with a series of human tumour cell lines. The results (shown as % inhibition of colony forming ability) obtained with cancer cells derived from the bladder, breast, lung, colon, pancreas, prostate, liver and cervix, are shown in Table 13. The specific cell lines employed are as follows:

T24=bladder cell carcinoma
HCT116=colon cell carcinoma
A549=lung cell carcinoma
MDA-MB-231=breast cell adenocarcinoma
MDA PaCa-2=pancreatic cell carcinoma
PC-3=prostrate cell adenocarcinoma
HepG2=hepatocellular carcinoma
HeLaS3=cells isolated from a carcinoma of the cervix
T-47D=breast ductal carcinoma
H596=lung adenosquamous carcinoma cells
Colo320=colon cell adenocarcinoma

TABLE 13
Reduced Relative Colony Forming Efficiency of Human Tumour Cells following Treatment
with Antisense Oligonucleotides Targeting Ribonucleotide Reductase R2

| | | | | MDA- | MIA | | | | | | |
|---|---|---|---|---|---|---|---|---|---|---|---|
| Name (Re) | T24 | HCT116 | A549 | MB-231 | PaCa-2 | PC-3 HepG2 | | Hela S3 | T-47D | 11596 | Co1o320 |
| AS-II-6-20 | 73.55 | ND | ND | 85.4 | 95.15 | 59.21 | 97.89 | ND | ND | ND | ND |
| AS-II-13-20 * | 18.99 | 6.95 | 32.3 | 45.45 | ND | 52.38 | 24.11 | 19.85 | 15.33 | 19.63 | ND |
| AS-II-14-20 | 77.59 | ND | ND | 91.24 | 47.93 | 92.76 | 88.4 | ND | ND | ND | ND |
| AS-II-16-18 | 25.74 | 78.57 | 81.1 | 62.59 | ND | 89.48 | 75.89 | 68.7 | 7.13 | 34.5 | ND |
| AS-II-75-20 * | 73.42 | 44.4 | 60.08 | 49.3 | 97.38 | 68.25 | 35.4 | 93.01 | 32.95 | ND | ND |
| AS-II-75-20 | 95.83 | ND | ND | 95.14 | 52.07 | 83.46 | 97.89 | ND | ND | ND | ND |
| AS-II-79-14 | 38.4 | 45.56 | 79.17 | 48.6 | 38.89 | 85.32 | 70.81 | 28.64 | 70.81 | ND | ND |
| AS-II-109-20 * | 24.89 | 6.76 | 15.14 | 22.38 | 54.24 | 61.51 | 18.08 | 46.83 | 20.63 | 7.28 | ND |
| AS-II-110-20 | 87.78 | 71.69 | 89.38 | 90.92 | 47.51 | 92.06 | 97.14 | 53.98 | ND | ND | ND |
| AS-II-114-20 | 87.45 | 86.1 | 83.51 | 76.22 | 90.05 | 92.66 | 78.72 | 79.25 | 90.83 | 46.3 | ND |
| AS-II-127-12 | 50.63 | 54.34 | 69.33 | 38.46 | 53.34 | 79.56 | 71.75 | 86.45 | 37.54 | ND | ND |
| AS-II-130-20 | 5194 | 5798 | 86.48 | ND | 82.11 | 74.66 | 94.28 | ND | ND | ND | ND |
| AS-II-134-20 | ND | ND | ND | ND | ND | 77.51 | ND | ND | ND | ND | ND |
| AS-II-151-20 | ND | 78.09 | 84.28 | 41.64 | 75.38 | 35.68 | 89.53 | 66.75 | 95.39 | 69.12 | 90.12 |
| AS-II-163-20 * | 5.49 | 29.05 | 37.13 | 22.73 | 9.88 | 7.14 | 18.64 | 45.8 | 9.81 | 32.08 | ND |
| AS-II-166-20 | 68.99 | 73.84 | 81.1 | 29.02 | 91.36 | 74.11 | 78.72 | 80.1 | 91.4 | 61.99 | ND |
| AS-II-185-20 | 21.94 | ND | 71.51 | 17.4 | 29.32 | 4.37 | 53.44 | 19.38 | 94.52 | 24.53 | ND |
| AS-II-189-20 | 18.57 | 86.78 | 76.57 | 39.86 | 70.52 | 73.12 | 57.86 | 76.67 | 96.63 | 26.15 | ND |
| AS-II-201-20 | 96.2 | 45.56 | 90.55 | 25.17 | 7U.22 | 65.08 | 59.32 | 90.87 | 98.53 | 49.6 | ND |
| AS-II-217-20 | 65.02 | 61.85 | ND | 52.7 | 87.38 | 87.41 | 99.55 | ND | ND | ND | ND |
| AS-II-225-20 | 73.23 | 59.5 | 929 | ND | 95.44 | 30.06 | ND | 9699 | ND | ND | ND |
| AS-II-253-14 | 19.41 | 53.28 | 61.62 | 4537 | ND | 67.26 | 42 | 65.18 | 27.09 | 0.81 | ND |
| AS-II-280-20 | 90.56 | 69.42 | 61.81 | 79.14 | 53.94 | 77.51 | 97.14 | 41.79 | ND | ND | ND |
| AS-II-288-12 | 30.38 | 67.57 | 70.49 | 52.1 | 30.09 | 74.01 | 65.89 | 57.63 | ND | 12.67 | ND |
| AS-II-323-20 | ND | 55.8 | 91.24 | ND | 97.55 | 79.76 | 96.39 | ND | ND | ND | ND |
| AS-II-344-20 | ND | ND | ND | ND | ND | 80.06 | ND | ND | ND | ND | ND |
| AS-II-362-20 | 89.63 | 62.81 | 61.81 | 85.83 | 34.2 | 75.78 | 95.78 | 45.69 | ND | ND | ND |
| AS-II-391-17 | ND | ND | ND | ND | 26.35 | 93.25 | 60.64 | ND | ND | ND | ND |
| AS-II-404-20 | 84.26 | ND | 52.17 | 85.83 | 17.84 | 77.08 | 84.79 | 58.37 | ND | ND | ND |
| AS-II-412-20 | 22.2 | 27.98 | 43.78 | ND | 55.25 | 73.96 | 26.23 | ND | ND | ND | ND |
| AS-II-414-20 | 11.67 | 19.1 | 12.44 | ND | 36.11 | 60.94 | 30.89 | ND | ND | ND | ND |
| AS-II-425-20 | 90.37 | ND | 57.38 | 89.75 | 65.2 | 75.65 | 97.89 | 63.09 | ND | ND | ND |
| AS-II-439-20 | 67.84 | 64.7 | 76.46 | ND | 92.69 | 77.66 | 73.04 | ND | ND | ND | ND |
| AS-II-472-20 | 69.26 | 67.23 | 96.99 | ND | 97.13 | 90.7 | ND | ND | ND | ND | ND |
| AS-II-494-20 | 54.23 | 50.28 | 33.85 | 54.78 | 25.31 | 30.6 | 93.37 | 43.62 | ND | ND | ND |
| AS-II-496-16 | 78.48 | 70.85 | 74.45 | 45.8 | ND | 88.84 | 54.8 | 52.21 | 10.79 | ND | ND |
| AS-II-549-20 | 45.46 | 47.83 | 30.57 | 40.13 | 17.01 | ND | 84.04 | 27.8 | ND | ND | ND |
| AS-II-579-20 | 76.68 | 69.08 | 95.49 | 66.89 | 97.55 | 88.16 | 94.28 | ND | ND | ND | ND |
| AS-II-619-20 | 86.3 | ND | 65.67 | 91.08 | 39.83 | 88.01 | 92.02 | 31.22 | ND | ND | ND |
| AS-II-626-20 | 76.79 | 70.46 | 95.14 | 90.21 | 75.62 | 83.23 | 75.89 | 67.92 | 66.12 | ND | ND |
| AS-II-634-20 | 83.52 | ND | 57.76 | 92.44 | ND | 77.86 | 95.78 | 48.94 | ND | ND | ND |
| AS-II-667-20 | 70.48 | 763 | 70.3 | ND | 85.26 | 91.8 | 88.23 | ND | ND | ND | ND |
| AS-II-784-20 | 87.23 | 78.09 | 83.8 | 33.92 | 62.04 | 88.99 | 80.89 | 81.48 | 85.39 | ND | ND |
| AS-II-798-20 | 84.72 | 64.46 | 70.49 | 83.92 | 34.65 | 83.21 | 89.46 | 56.42 | NO | ND | ND |
| AS-II-816-20 | 73.91 | 88.22 | 78.4 | ND | 93.21 | 94.08 | 93.08 | ND | ND | ND | ND |
| AS-II-861-20 | 73.5 | 74.2 | 95.78 | 89.98 | 97.3 | 87.33 | 96.08 | ND | ND | ND | ND |
| AS-II-890-20 | 82.07 | ND | 81.6 | 88.2 | 66.02 | 87.93 | ND | ND | ND | ND | ND |
| AS-II-909-20 | 78.57 | ND | 78.68 | 45.96 | 46.13 | 84.86 | ND | ND | ND | ND | ND |
| AS-II-933-20 | 64.84 | 67.24 | 53.52 | 64.39 | 35.68 | 36.91 | 7997 | 26.86 | ND | ND | ND |
| AS-II-981-20 | 86.3 | 66.84 | 74.25 | 91.48 | ND | 85.16 | 95.03 | 69.43 | ND | ND | ND |
| AS-II-1001-20 | 86.11 | 55.58 | 71.36 | 82.17 | 64.21 | 85.94 | 90.36 | ND | ND | ND | ND |
| AS-II-1006-20 | 61.49 | 45.56 | 61.62 | ND | 47.93 | 92.58 | 89.31 | 41.79 | ND | ND | ND |
| AS-II-1023-20 | 58.26 | ND | 34.52 | ND | 42.82 | 87.63 | ND | ND | ND | ND | ND |
| AS-II-1040-20 | 59.49 | 70.08 | 85.82 | ND | 43.52 | 40.08 | 77.78 | 71.87 | 64.76 | ND | ND |
| AS-II-1048-20 | 40.32 | 42.63 | 65.67 | 66.88 | 33.4 | 84.38 | 77.56 | 39.19 | ND | ND | ND |
| AS-II-114420 | 62.9 | 54.25 | 61.81 | ND | 46.39 | 30.21 | 92.17 | 50.57 | ND | ND | ND |
| AS-II-1182-20 | 94.51 | 88.13 | 80.06 | ND | 84.72 | 92.76 | 92.23 | 90.61 | 92.41 | ND | ND |
| AS-II-1197-20 | 90.3 | 84.85 | 89.15 | 50.35 | 70.68 | 74.4 | 76.32 | 82.68 | 81.95 | ND | ND |
| AS-II-1217-20 | 66.36 | 68.68 | 91.49 | ND | 34.85 | 81.03 | ND | ND | ND | ND | ND |
| AS-II-1224-20 | 38.31 | 41.78 | 55.06 | ND | 17.22 | 80.66 | 76.05 | 14.8 | ND | ND | ND |
| AS-II-1254-20 | 41.53 | 28.54 | 36.74 | ND | 3.32 | 73.31 | 83.28 | 7.64 | ND | ND | ND |
| AS-II-1278-20 | 65.42 | ND | ND | 90.68 | 57.05 | 85.31 | ND | ND | ND | ND | ND |
| AS-II-1283-20 | 56.75 | 66.43 | 61.04 | ND | 80.71 | 93.55 | 30.41 | ND | ND | ND | ND |
| AS-II-1302-20 | 70.56 | 71.98 | 93.17 | 92.2 | 23.86 | 79.01 | ND | ND | ND | ND | ND |
| AS-II-1335-20 | 59.95 | 67.87 | 78.59 | ND | 78.78 | 90.04 | 72.98 | ND | ND | ND | ND |
| AS-II-1338-20 | 63.16 | 74.73 | 63.93 | ND | 79.17 | 93.75 | 80.41 | ND | ND | ND | ND |
| AS-II-134220 | 59.76 | 73.74 | 65.67 | ND | 73.77 | 89.84 | 82.2 | ND | ND | ND | ND |
| AS-II-1345-20 | 51.26 | 65.7 | 73.1 | 94.11 | 77.39 | 89.58 | 75.42 | ND | ND | ND | ND |
| AS-II-1362-20 | ND | 78.47 | 83.9 | 70.22 | 44.14 | 77.38 | 80.41 | ND | ND | ND | ND |
| AS-II-1364-20 | 66.59 | 77.29 | 95.59 | 93.87 | 59.34 | 79.01 | ND | ND | ND | ND | ND |
| AS-II-1381-20 | 71.37 | 89.48 | 86.02 | 44.41 | 73.77 | 75 | 62.34 | 80.53 | 93.62 | 4528 | ND |
| AS-II-1390-20 | 61.13 | 62.18 | 88.31 | 66.89 | 82.77 | 76.76 | 90.21 | ND | ND | ND | ND |
| AS-II-1438-20 | 43.7 | ND | 51.27 | 69.06 | 42.13 | 83.96 | ND | ND | ND | ND | ND |
| AS-II-1499-20 | 82.81 | 83.01 | 87.8 | 41.26 | 81.17 | 77.28 | 77.5 | 87.56 | 96.67 | 78.3 | ND |
| AS-II-1517-20 | ND | ND | ND | ND | ND | 91.75 | ND | ND | ND | ND | ND |
| AS-II-1538-20 | 67.29 | 51.28 | 90.34 | ND | 50.62 | 84.71 | 96.84 | ND | ND | ND | ND |
| AS-II-1560.20 | 32.49 | 85.81 | 84.19 | 46.15 | 33.3 | 78.37 | 73.63 | 82.16 | 86.6 | 71.16 | ND |
| AS-II-1581-20 | 68.22 | 66.85 | 90.55 | ND | 24.07 | 85.83 | 93.07 | ND | ND | ND | ND |
| AS-II-1659-20 | 74.09 | ND | 54.7 | 42.86 | 42.54 | 81.56 | ND | ND | ND | ND | ND |
| AS-II-1666-20 | 71.71 | ND | 54.82 | 26.71 | 49.72 | 86.06 | ND | ND | ND | ND | ND |
| AS-II-1700-20 | 70.94 | ND | 77.28 | 30.75 | 34.52 | 90.63 | ND | ND | ND | ND | ND |
| AS-II-1768-20 | 74.56 | ND | 86.8 | 91.56 | 60.36 | 86.36 | ND | ND | ND | ND | ND |
| AS-II-1773-20 | 15.19 | 75.58 | 70.11 | 44.76 | 45.68 | 70.04 | 58.19 | 80.27 | 84.38 | 66.04 | ND |
| AS-II-1775-12 | 85.54 | 5444 | 63.55 | 48.6 | 27.78 | 78.17 | 43.97 | 68.61 | ND | 18.6 | ND |
| AS-II-1790-2U | ND | ND | ND | ND | ND | 87.86 | ND | ND | ND | ND | ND |
| AS-II-1819-20 | 53.74 | ND | ND | 90.68 | 20.02 | 85.46 | 83.89 | ND | ND | ND | ND |
| AS-II-1976-20 | ND | ND | ND | 89.6 | ND | 88.16 | ND | ND | ND | ND | ND |
| AS-II-1989-20 | 77.43 | 78.47 | 83.9 | 54.9 | 70.22 | 77.38 | 80.7 | 61.41 | 90.83 | 56.33 | ND |
| AS-II-2009-20 | 61.84 | 69.92 | 93.32 | 96.25 | 93.74 | 83.36 | 96.99 | ND | ND | ND | ND |
| AS-II-2026-20 | 95.46 | 81.47 | 88.81 | 77.1 | 87.65 | 95.29 | 94.54 | 83.79 | 93.41 | 84.16 | ND |
| AS-II-2044-20 | 53.63 | 49.34 | 25.55 | 19.11 | 24.48 | 74.48 | 62.35 | 24.55 | ND | ND | ND |
| AS-II-2067-20 | 49.6 | 47.16 | 64.71 | 49.68 | 41.08 | 8594 | 90.36 | 24.88 | ND | ND | ND |
| AS-II-2083-20 | 82.43 | 87.46 | 90.65 | 68.88 | 71 | ND | 93.64 | 84.58 | 89.32 | 82.98 | 87.28 |
| AS-II-2083-20 * | 9.52 | 41.16 | 31.73 | ND | ND | 32.03 | 46.14 | 696 | 43.61 | 49.37 | 52.54 |
| AS-II-2128-20 | 83.74 | ND | 87.31 | 91.3 | 39.23 | 88.89 | ND | ND | ND | ND | ND |
| AS-II-2151-20 | 79.83 | ND | 79.19 | 95.14 | 62.15 | 84.86 | ND | ND | ND | ND | ND |
| AS-II-2164-20 | 61.84 | 50.08 | 91.15 | 69.03 | 89.36 | 83.36 | 93.07 | ND | ND | ND | ND |
| AS-II-218220 | 67.76 | 77.66 | 90.97 | 84.95 | 56.43 | 85.91 | 95.48 | ND | ND | ND | ND |
| AS-II-2229A-20 | 50.34 | 93.01 | 69.72 | ND | 33.61 | 89.58 | 73.26 | 63.15 | 58.82 | ND | ND |
| AS-II-2372-20 | 61.13 | 64.7 | 96.41 | 90.09 | 94.36 | 86.06 | ND | ND | ND | ND | ND |

Legend to Table 13
The antisense oligonucleotides were fully thioated unless indicated *, as described in Table 1
The values for relative colony-forming efficencies are averages obtained from 2-8 determinations.
ND = not determined.
The various cell lines were obtained from the American Type Culture Collection, Rockville Maryland.

### Example 13

### Sensitization of Human Tumour Cells to the Effects of Chemotherapeutics by Antisense Oligonucleotides Targeted to Ribonucleotide Reductase R2

Treatment of human tumour cells with very low concentrations of short antisense sequences was tested to determine if these constructs could sensitize the tumour cells to inhibitory effects of other chemotherapeutic drugs. The concentration used was not cytotoxic in itself (as demonstrated by the results shown in Table 11). The treatment of Hela S3 and Hela 1 mM cells with 0.02 μM of the AS-II-2229B-20 antisense oligonucleotide increased the sensitivity of these cells to N-(phosphonacetyl)-L-aspartate (PALA) and to methotrexate (MTX) as shown in Table 14.

TABLE 14
Sensitization Effect of AS-II-2229B-20 as Antisense Construct

| | | Drug | AS-II-2229B-20 | Relative colony |
|---|---|---|---|---|
| Cells | Drug | Conc. | 0.02 μM | Forming Efficiency* |
| HeLaS3 | PALA | 20 μM | − | 350 ± 50 |
| | PALA | 20 μM | + | 90 ± 10 |
| HeLa S3 | MTX | 40 μM | − | 118 ± 32 |
| | MTX | 60 μM | − | 116 ± 13 |
| | MTX | 40 μM | + | 25 ± 5 |
| | MTX | 60 μM | + | 0 |
| HeLa | PALA | 20 μM | − | 377 ± 21 |
| 1 mM | PALA | 30 μM | − | 311 ± 9.5 |
| | PALA | 20 μM | + | 108 ± 7.5 |
| | PALA | 30 μM | + | 101 ± 2.0 |
| HeLa | MTX | 40 μM | − | 28 ± 10 |
| 1 mM | MTX | 60 μM | − | 12 ± 0.5 |
| | MTX | 40 μM | + | 6.5 ± 5.5 |
| | MTX | 60 μM | + | 3.5 ± 0.5 |

− = no treatment
+ = treatment provided
*The values are the average of two experiments

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

The embodiments of the invention in which an exclusive property or privilege is claimed are defined as follows:

1. A combination product for use in the treatment of cancer in a mammal, said combination product comprising: an antisense oligonucleotide of between 7 and 100 nucleotides in length comprising at least 7 consecutive nucleotides complementary to a mammalian ribonucleotide reductase R2 subunit mRNA and one or more immunotherapeutic agents.

2. The combination product according to claim 1, wherein said mammalian ribonucleotide reductase R2 subunit mRNA is a human ribonucleotide reductase R2 subunit mRNA.

3. The combination product according to claim 2, wherein said human ribonucleotide reductase R2 subunit mRNA has a sequence as set forth in SEQ ID NO:105.

4. The combination product according to claim 2, wherein said antisense oligonucleotide comprises at least 7 consecutive nucleotides of the sequence as set forth in any one of SEQ ID NOs:1 and 4-104.

5. The combination product according to according to claim 2, wherein said antisense oligonucleotide comprises at least 7 consecutive nucleotides of the sequence as set forth in SEQ ID NO:1.

6. The combination product according to any one of claims 1 to 5, wherein said antisense oligonucleotide comprises one or more phosphorothioate internucleotide linkages.

7. The combination product according to any one of claims 1 to 6, wherein said cancer is an advanced cancer.

8. The combination product according to any one of claims 1 to 7, wherein said cancer is a metastatic cancer.

9. The combination product according to any one of claims 1 to 8, wherein said treatment is a first-line systemic therapy.

10. The combination product according to any one of claims 1 to 9, wherein said one or more immunotherapeutic agents are non-specific immunotherapeutic agents.

11. The combination product according to any one of claims 1 to 9, wherein said one or more immunotherapeutic agents are specific immunotherapeutic agents.

12. The combination product according to any one of claims 1 to 10, wherein said one or more immunotherapeutic agents are selected from the group of: a cytokine, a non-cytokine adjuvant, a monoclonal antibody and a cancer vaccine.

13. The combination product according to any one of claims 1 to 10, wherein said one or more immunotherapeutic agents are selected from the group of: a cytokine and a non-cytokine adjuvant.

14. The combination product according to any one of claims 1 to 10, wherein said one or more immunotherapeutic agents are one or more cytokines.

15. The combination product according to any one of claims 1 to 14, wherein said combination product further comprises one or more chemotherapeutic agents.

16. The combination product according to any one of claims 1 to 15, wherein said cancer is a solid cancer.

17. The combination product according to any one of claims 1 to 16, wherein said mammal is a human.

18. A method of treating cancer in a mammal comprising administering to said mammal a combination product comprising:
(a) an antisense oligonucleotide of between 7 and 100 nucleotides in length comprising at least 7 consecutive nucleotides complementary to a mammalian ribonucleotide reductase R2 subunit mRNA, and
(b) one or more immunotherapeutic agents.

19. The method according to claim 18, wherein said mammalian ribonucleotide reductase R2 subunit mRNA is a human ribonucleotide reductase R2 subunit mRNA.

20. The combination product according to claim 19, wherein said human ribonucleotide reductase R2 subunit mRNA has a sequence as set forth in SEQ ID NO:105.

21. The method according to claim 19, wherein said antisense oligonucleotide comprises at least 7 consecutive nucleotides of the sequence as set forth in any one of SEQ ID NOs:1 and 4-104.

22. The method according to according to claim 19, wherein said antisense oligonucleotide comprises at least 7 consecutive nucleotides of the sequence as set forth in SEQ ID NO:1.

23. The method according to any one of claims 18 to 22, wherein said antisense oligonucleotide comprises one or more phosphorothioate internucleotide linkages.

24. The method according to any one of claims 18 to 23, wherein said cancer is an advanced cancer.

25. The method according to any one of claims 18 to 24, wherein said cancer is a metastatic cancer.

26. The method according to any one of claims 18 to 25, wherein said combination product is administered to said mammal as first-line systemic therapy.

27. The method according to any one of claims 18 to 26, wherein said one or more immunotherapeutic agents are non-specific immunotherapeutic agents.

28. The method according to any one of claims 18 to 26, wherein said one or more immunotherapeutic agents are specific immunotherapeutic agents.

29. The method according to any one of claims 18 to 27, wherein said one or more immunotherapeutic agents are selected from the group of: a cytokine, a non-cytokine adjuvant, a monoclonal antibody and a cancer vaccine.

30. The method according to any one of claims 18 to 27, wherein said one or more immunotherapeutic agents are selected from the group of: a cytokine and a non-cytokine adjuvant

31. The method according to any one of claims 18 to 27, wherein said one or more immunotherapeutic agents are one or more cytokines.

32. The method according to any one of claims 18 to 31, wherein said combination product further comprises one or more chemotherapeutic agents.

33. The method according to any one of claims 18 to 32, wherein said cancer is a solid cancer.

34. The method according to any one of claims 18 to 33, wherein said mammal is a human.

35. Use of an antisense oligonucleotide of between 7 and 100 nucleotides in length comprising at least 7 consecutive nucleotides complementary to a mammalian ribonucleotide reductase R2 subunit mRNA and one or more immunotherapeutic agents in the manufacture of a medicament for the treatment of cancer in a mammal.

36. The use according to claim 35, wherein said mammalian ribonucleotide reductase R2 subunit mRNA is a human ribonucleotide reductase R2 subunit mRNA.

37. The use according to claim 36, wherein said human ribonucleotide reductase R2 subunit mRNA has a sequence as set forth in SEQ ID NO:105.

38. The use according to claim 36, wherein said antisense oligonucleotide comprises at least 7 consecutive nucleotides of the sequence as set forth in any one of SEQ ID NOs:1 and 4-104.

39. The use according to claim 36, wherein said antisense oligonucleotide comprises at least 7 consecutive nucleotides of the sequence as set forth in SEQ ID NO:1.

40. The use according to any one of claims 35 to 39, wherein said antisense oligonucleotide comprises one or more phosphorothioate internucleotide linkages.

41. The use according to any one of claims 35 to 40, wherein said cancer is an advanced cancer.

42. The use according to any one of claims 35 to 41, wherein said cancer is a metastatic cancer.

43. The use according to any one of claims 35 to 42, wherein said treatment is a first-line systemic therapy.

44. The use according to any one of claims 35 to 43, wherein said one or more immunotherapeutic agents are non-specific immunotherapeutic agents.

45. The use according to any one of claims 35 to 43, wherein said one or more immunotherapeutic agents are specific immunotherapeutic agents.

46. The use according to any one of claims 35 to 44, wherein said one or more immunotherapeutic agents are selected from the group of: a cytoidne, a non-cytokine adjuvant, a monoclonal antibody and a cancer vaccine.

47. The use according to any one of claims 35 to 44, wherein said one or more immunotherapeutic agents are selected from the group of: a cytokine and a non-cytokine adjuvant.

48. The use according to any one of claims 35 to 44, wherein said one or more immunotherapeutic agents are one or more cytokines.

49. The use according to any one of claims 35 to 48, wherein said combination product further comprises one or more chemotherapeutic agents.

50. The use according to any one of claims 35 to 49, wherein said cancer is a solid cancer.

51. The use according to any one of claims 35 to 50, wherein said mammal is a human.

52. A pharmaceutical kit comprising a combination product for the treatment of cancer, said combination product comprising:
(a) an antisense oligonucleotide of between 7 and 100 nucleotides in length comprising at least 7 consecutive nucleotides complementary to a mammalian ribonucleotide reductase R2 subunit mRNA, and
(b) one or more immunotherapeutic agents.

53. A combination product for use in the treatment, of renal cancer in a subject, said combination product comprising: an antisense oligonucleotide of between 7 and 100 nucleotides in length comprising at least 7 consecutive nucleotides complementary to SEQ ID NO:1 and one or more cytokines.

54. The combination product according to claim 53, wherein said one or more cytokines are selected from: interferon alpha and interleukin-2.

55. The combination product according to claim 53 or 54, wherein said treatment is a first-line systemic therapy.


SEQUENCE LISTING (105 sequences)

SEQ ID NO: 1 - DNA, 20 bp, Artificial Sequence
  Antisense oligonucleotide complementary to human ribonucleotide reductase R2 mRNA
ggctaaatcgctccaccaag

SEQ ID NO: 2 - DNA, 20 bp, Artificial Sequence
  Control antisense oligonucleotide
ggctaaactcgtccaccaag

SEQ ID NO: 3 - DNA, 20 bp, Artificial Sequence
  control oligonucleotide
acgcactcagctagtgacac

SEQ ID NO: 4 - DNA, 20 bp, Artificial Sequence
  AS-II-6-20 antisense oligonucleotide complementary to human ribonucleotide reductase R2 mRNA
acccttcccattggctgcgc

SEQ ID NO: 5 - DNA, 20 bp, Artificial Sequence
  AS-II-13-20 antisense oligonucleotides complementary to human ribonucleotide reductase R2 mRNA
gcctccgacccttcccattg

SEQ ID NO: 6 - DNA, 20 bp, Artificial Sequence
  AS-II-14-20 antisense oligonucleotides complementary to human ribonucleotide reductase R2 mRNA
tgcctccgacccttcccatt

SEQ ID NO: 7 - DNA, 18 bp, Artificial Sequence
  AS-II-16-18 antisense oligonucleotides complementary to human ribonucleotide reductase R2 mRNA
tgcctccgacccttccca

SEQ ID NO: 8 - DNA, 20 bp, Artificial Sequence
  Partially Phosphorothiolated AS-II-75-20 antisenseoligonucleotides complementary to human ribonucleotide reductaseR2 mRNA
cgcgcgctcccggcccttcc

SEQ ID NO: 9 - DNA, 20 bp, Artificial Sequence
  AS-II-75-20 antisense oligonucleotides complementary to human ribonucleotide reductase R2 mRNA
cgcgcgctcccggcccttcc

SEQ ID NO: 10 - DNA, 14 bp, Artificial Sequence
  AS-II-79-14 antisense oligonucleotides complementary to human ribonucleotide reductase R2 mRNA
cgcgctcccggccc

SEQ ID NO: 11 - DNA, 20 bp, Artificial Sequence
  AS-II-109-20 antisense oligonucleotides complementary to human ribonucleotide reductase R2 mRNA
cccctcactccagcagcctt

SEQ ID NO: 12 - DNA, 20 bp, Artificial Sequence
  AS-II-110-20 antisense oligonucleotides complementary to human ribonucleotide reductase R2 mRNA
acccctcactccagcagcct

SEQ ID NO: 13 - DNA, 20 bp, Artificial Sequence
  AS-II-114-20 antisense oligonucleotides complementary to human ribonucleotide reductase R2 mRNA
ggcgacccctcactccagca

SEQ ID NO: 14 - DNA, 12 bp, Artificial Sequence
  AS-II-127-12 antisense oligonucleotides complementary to human ribonucleotide reductase R2 mRNA
gcacgggcgacc

SEQ ID NO: 15 - DNA, 20 bp, Artificial Sequence
  AS-II-130-20 antisense oligonucleotides complementary to human ribonucleotide reductase R2 mRNA
tgggacagggtgcacgggcg

SEQ ID NO: 16 - DNA, 20 bp, Artificial Sequence
  AS-II-134-20 antisense oligonucleotides complementary to human ribonucleotide reductase R2 mRNA
gacggctgggacagggtgca

SEQ ID NO: 17 - DNA, 20 bp, Artificial Sequence
  AS-II-151-20 antisense oligonucleotides complementary to human ribonucleotide reductase R2 mRNA
gagcagccaggacaggacgg

SEQ ID NO: 18 - DNA, 20 bp, Artificial Sequence
  AS-II-163-20 antisense oligonucleotides complementary to human ribonucleotide reductase R2 mRNA
gcgaagcagagcgagcagcc

SEQ ID NO: 19 - DNA, 20 bp, Artificial Sequence
  AS-II-166-20 antisense oligonucleotides complementary to human ribonucleotide reductase R2 mRNA
gcagcgaagcagagcgagca

SEQ ID NO: 20 - DNA, 20 bp, Artificial Sequence
  AS-II-185-20 antisense oligonucleotides complementary to human ribonucleotide reductase R2 mRNA
gggagagcatagtggaggcg

SEQ ID NO: 21 - DNA, 20 bp, Artificial Sequence
  AS-II-189-20 antisense oligonucleotides complementary to human ribonucleotide reductase R2 mRNA
cggagggagagcatagtgga

SEQ ID NO: 22 - DNA, 20 bp, Artificial Sequence
  AS-II-201-20 antisense oligonucleotides complementary to human ribonucleotide reductase R2 mRNA
gcgagcgggacacggaggga

SEQ ID NO: 23 - DNA, 20 bp, Artificial Sequence
  AS-II-217-20 antisense oligonucleotides complementary to human ribonucleotide reductase R2 mRNA
cgggtccgtgatgggcgcga

SEQ ID NO: 24 - DNA, 20 bp, Artificial Sequence
  AS-II-225-20 antisense oligonucleotides complementary to human ribonucleotide reductase R2 mRNA
agctgctgcgggtccgtgat

SEQ ID NO: 25 - DNA, 14 bp, Artificial Sequence
  AS-II-253-14 antisense oligonucleotides complementary to human ribonucleotide reductase R2 mRNA
ccccttcagcggcg

SEQ ID NO: 26 - DNA, 20 bp, Artificial Sequence
  AS-II-280-20 antisense oligonucleotides complementary to human ribonucleotide reductase R2 mRNA
cggcggcgtgttctccttgt

SEQ ID NO: 27 - DNA, 12 bp, Artificial Sequence
  AS-II-288-12 antisense oligonucleotides complementary to human ribonucleotide reductase R2 mRNA
cggcggcgtgtt

SEQ ID NO: 28 - DNA, 20 bp, Artificial Sequence
  AS-II-323-20 antisense oligonucleotides complementary to human ribonucleotide reductase R2 mRNA
tcctcgcggtcttgctggcc

SEQ ID NO: 29 - DNA, 20 bp, Artificial Sequence
  AS-II-344-20 antisense oligonucleotides complementary to human ribonucleotide reductase R2 mRNA
ccgtgggctcctggaagatc

SEQ ID NO: 30 - DNA, 20 bp, Artificial Sequence
  AS-II-362-20 antisense oligonucleotides complementary to human ribonucleotide reductase R2 mRNA
ctgctttagttttcggctcc

SEQ ID NO: 31 - DNA, 17 bp, Artificial Sequence
  AS-II-391-17 antisense oligonucleotides complementary to human ribonucleotide reductase R2 mRNA
cggctcatcctccacgc

SEQ ID NO: 32 - DNA, 20 bp, Artificial Sequence
  AS-II-404-20 antisense oligonucleotides complementary to human ribonucleotide reductase R2 mRNA
ggttttctctcagcagcggc

SEQ ID NO: 33 - DNA, 20 bp, Artificial Sequence
  AS-II-412-20 antisense oligonucleotides complementary to human ribonucleotide reductase R2 mRNA
gcggcgggggttttctctca

SEQ ID NO: 34 - DNA, 20 bp, Artificial Sequence
  AS-II-414-20 antisense oligonucleotides complementary to human ribonucleotide reductase R2 mRNA
aagcggcgggggttttctct

SEQ ID NO: 35 - DNA, 20 bp, Artificial Sequence
  AS-II-425-20 antisense oligonucleotides complementary to human ribonucleotide reductase R2 mRNA
ggaagatgacaaagcggcgg

SEQ ID NO: 36 - DNA, 20 bp, Artificial Sequence
  AS-II-439-20 antisense oligonucleotides complementary to human ribonucleotide reductase R2 mRNA
atggtactcgatggggaaga

SEQ ID NO: 37 - DNA, 20 bp, Artificial Sequence
  AS-II-472-20 antisense oligonucleotides complementary to human ribonucleotide reductase R2 mRNA
agcctctgccttcttataca

SEQ ID NO: 38 - DNA, 20 bp, Artificial Sequence
  AS-II-494-20 antisense oligonucleotides complementary to human ribonucleotide reductase R2 mRNA
cctcctcggcggtccaaaag

SEQ ID NO: 39 - DNA, 16 bp, Artificial Sequence
  AS-II-496-16 antisense oligonucleotides complementary to human ribonucleotide reductase R2 mRNA
tcctcggcggtccaaa

SEQ ID NO: 40 - DNA, 20 bp, Artificial Sequence
  AS-II-549-20 antisense oligonucleotides complementary to human ribonucleotide reductase R2 mRNA
tatctctcctcgggtttcag

SEQ ID NO: 41 - DNA, 20 bp, Artificial Sequence
  AS-II-579-20 antisense oligonucleotides complementary to human ribonucleotide reductase R2 mRNA
gcaaagaaagccagaacatg

SEQ ID NO: 42 - DNA, 20 bp, Artificial Sequence
  AS-II-619-20 antisense oligonucleotides complementary to human ribonucleotide reductase R2 mRNA
tcgctccaccaagttttcat

SEQ ID NO: 43 - DNA, 20 bp, Artificial Sequence
  AS-II-634-20 antisense oligonucleotides complementary to human ribonucleotide reductase R2 mRNA
aacttcttggctaaatcgct

SEQ ID NO: 44 - DNA, 20 bp, Artificial Sequence
  AS-II-667-20 antisense oligonucleotides complementary to human ribonucleotide reductase R2 mRNA
gaagccatagaaacagcggg

SEQ ID NO: 45 - DNA, 20 bp, Artificial Sequence
  AS-II-784-20 antisense oligonucleotides complementary to human ribonucleotide reductase R2 mRNA
gacacaaggcatcgtttcaa

SEQ ID NO: 46 - DNA, 20 bp, Artificial Sequence
  AS-II-798-20 antisense oligonucleotides complementary to human ribonucleotide reductase R2 mRNA
tctgccttcttcttgacaca

SEQ ID NO: 47 - DNA, 20 bp, Artificial Sequence
  AS-II-816-20 antisense oligonucleotides complementary to human ribonucleotide reductase R2 mRNA
atccagcgcaaggcccagtc

SEQ ID NO: 48 - DNA, 20 bp, Artificial Sequence
  AS-II-861-20 antisense oligonucleotides complementary to human ribonucleotide reductase R2 mRNA
gcaaaggctacaacacgttc

SEQ ID NO: 49 - DNA, 20 bp, Artificial Sequence
  AS-II-890-20 antisense oligonucleotides complementary to human ribonucleotide reductase R2 mRNA
aaccggaaaagaaaatgcct

SEQ ID NO: 50 - DNA, 20 bp, Artificial Sequence
  AS-II-909-20 antisense oligonucleotides complementary to human ribonucleotide reductase R2 mRNA
ggcatcagtcctcgtttctt

SEQ ID NO: 51 - DNA, 20 bp, Artificial Sequence
  AS-II-933-20 antisense oligonucleotides complementary to human ribonucleotide reductase R2 mRNA
ggcatcagtcctcgtttctt

SEQ ID NO: 52 - DNA, 20 bp, Artificial Sequence
  AS-II-981-20 antisense oligonucleotides complementary to human ribonucleotide reductase R2 mRNA
tgtaaaccctcatctctgct

SEQ ID NO: 53 - DNA, 20 bp, Artificial Sequence
  AS-II-1001-20 antisense oligonucleotides complementary to human ribonucleotide reductase R2 mRNA
tcaggcaagcaaaatcacag

SEQ ID NO: 54 - DNA, 20 bp, Artificial Sequence
  AS-II-1006-20 antisense oligonucleotides complementary to human ribonucleotide reductase R2 mRNA
gaacatcaggcaagcaaaat

SEQ ID NO: 55 - DNA, 20 bp, Artificial Sequence
  AS-II-1023-20 antisense oligonucleotides complementary to human ribonucleotide reductase R2 mRNA
ttgtgtaccaggtgtttgaa

SEQ ID NO: 56 - DNA, 20 bp, Artificial Sequence
  AS-II-1040-20 antisense oligonucleotides complementary to human ribonucleotide reductase R2 mRNA
ctctctcctccgatggtttg

SEQ ID NO: 57 - DNA, 20 bp, Artificial Sequence
  AS-II-1048-20 antisense oligonucleotides complementary to human ribonucleotide reductase R2 mRNA
ttctcttactctctcctccg

SEQ ID NO: 58 - DNA, 20 bp, Artificial Sequence
  AS-II-1144-20 antisense oligonucleotides complementary to human ribonucleotide reductase R2 mRNA
gtattgcttcattagagtgc

SEQ ID NO: 59 - DNA, 20 bp, Artificial Sequence
  AS-II-1182-20 antisense oligonucleotides complementary to human ribonucleotide reductase R2 mRNA
cccagttccagcataagtct

SEQ ID NO: 60 - DNA, 20 bp, Artificial Sequence
  AS-II-1197-20 antisense oligonucleotides complementary to human ribonucleotide reductase R2 mRNA
aaaaccttgctaaaacccag

SEQ ID NO: 61 - DNA, 20 bp, Artificial Sequence
  AS-II-1217-20 antisense oligonucleotides complementary to human ribonucleotide reductase R2 mRNA
caaatgggttctctactctg

SEQ ID NO: 62 - DNA, 20 bp, Artificial Sequence
  AS-II-1224-20 antisense oligonucleotides complementary to human ribonucleotide reductase R2 mRNA
ataaagtcaaatgggttctc

SEQ ID NO: 63 - DNA, 20 bp, Artificial Sequence
  AS-II-1254-20 antisense oligonucleotides complementary to human ribonucleotide reductase R2 mRNA
ttagtctttccttccagtga

SEQ ID NO: 64 - DNA, 20 bp, Artificial Sequence
  AS-II-1278-20 antisense oligonucleotides complementary to human ribonucleotide reductase R2 mRNA
tcgcctactctcttctcaaa

SEQ ID NO: 65 - DNA, 20 bp, Artificial Sequence
  AS-II-1288-20 antisense oligonucleotides complementary to human ribonucleotide reductase R2 mRNA
cctctgatactcgcctactc

SEQ ID NO: 66 - DNA, 20 bp, Artificial Sequence
  AS-II-1302-20 antisense oligonucleotides complementary to human ribonucleotide reductase R2 mRNA
gacatcactcccatcctctg

SEQ ID NO: 67 - DNA, 20 bp, Artificial Sequence
  AS-II-1335-20 antisense oligonucleotides complementary to human ribonucleotide reductase R2 mRNA
gcatccaaggtaaaagaatt

SEQ ID NO: 68 - DNA, 20 bp, Artificial Sequence
  AS-II-1338-20 antisense oligonucleotides complementary to human ribonucleotide reductase R2 mRNA
tcagcatccaaggtaaaaga

SEQ ID NO: 69 - DNA, 20 bp, Artificial Sequence
  AS-II-1342-20 antisense oligonucleotides complementary to human ribonucleotide reductase R2 mRNA
gaagtcagcatccaaggtaa

SEQ ID NO: 70 - DNA, 20 bp, Artificial Sequence
  AS-II-1345-20 antisense oligonucleotides complementary to human ribonucleotide reductase R2 mRNA
ttagaagtcagcatccaagg

SEQ ID NO: 71 - DNA, 20 bp, Artificial Sequence
  AS-II-1362-20 antisense oligonucleotides complementary to human ribonucleotide reductase R2 mRNA
gcacatcttcagttcattta

SEQ ID NO: 72 - DNA, 20 bp, Artificial Sequence
  AS-II-1364-20 antisense oligonucleotides complementary to human ribonucleotide reductase R2 mRNA
gggcacatcttcagttcatt

SEQ ID NO: 73 - DNA, 20 bp, Artificial Sequence
  AS-II-1381-20 antisense oligonucleotides complementary to human ribonucleotide reductase R2 mRNA
aaaaatcagccaagtaaggg

SEQ ID NO: 74 - DNA, 20 bp, Artificial Sequence
  AS-II-1390-20 antisense oligonucleotides complementary to human ribonucleotide reductase R2 mRNA
atggaaaaaaaaaatcagcc

SEQ ID NO: 75 - DNA, 20 bp, Artificial Sequence
  AS-II-1438-20 antisense oligonucleotides complementary to human ribonucleotide reductase R2 mRNA
ttcatggtgtggctagttgg

SEQ ID NO: 76 - DNA, 20 bp, Artificial Sequence
  AS-II-1499-20 antisense oligonucleotides complementary to human ribonucleotide reductase R2 mRNA
aggactggttgtgaggtagc

SEQ ID NO: 77 - DNA, 20 bp, Artificial Sequence
  AS-II-1517-20 antisense oligonucleotides complementary to human ribonucleotide reductase R2 mRNA
ccagcactataaacagacag

SEQ ID NO: 78 - DNA, 20 bp, Artificial Sequence
  AS-II-1538-20 antisense oligonucleotides complementary to human ribonucleotide reductase R2 mRNA
ttctggcaaaaggtgatact

SEQ ID NO: 79 - DNA, 20 bp, Artificial Sequence
  AS-II-1560-20 antisense oligonucleotides complementary to human ribonucleotide reductase R2 mRNA
gtaagtcacagccagccagg

SEQ ID NO: 80 - DNA, 20 bp, Artificial Sequence
  AS-II-1581-20 antisense oligonucleotides complementary to human ribonucleotide reductase R2 mRNA
actgccattgtcactgctat

SEQ ID NO: 81 - DNA, 20 bp, Artificial Sequence
  AS-II-1659-20 antisense oligonucleotides complementary to human ribonucleotide reductase R2 mRNA
tggctgtgctggttaaagga

SEQ ID NO: 82 - DNA, 20 bp, Artificial Sequence
  AS-II-1666-20 antisense oligonucleotides complementary to human ribonucleotide reductase R2 mRNA
ttttaactggctgtgctggt

SEQ ID NO: 83 - DNA, 20 bp, Artificial Sequence
  AS-II-1700-20 antisense oligonucleotides complementary to human ribonucleotide reductase R2 mRNA
attaaaatctgcgttgaagc

SEQ ID NO: 84 - DNA, 20 bp, Artificial Sequence
  AS-II-1768-20 antisense oligonucleotides complementary to human ribonucleotide reductase R2 mRNA
tatcgccgccgtgagtacaa

SEQ ID NO: 85 - DNA, 20 bp, Artificial Sequence
  AS-II-1773-20 antisense oligonucleotides complementary to human ribonucleotide reductase R2 mRNA
gctattatcgccgccgtgag

SEQ ID NO: 86 - DNA, 12 bp, Artificial Sequence
  AS-II-1775-20 antisense oligonucleotides complementary to human ribonucleotide reductase R2 mRNA
atcgccgccgtg

SEQ ID NO: 87 - DNA, 20 bp, Artificial Sequence
  AS-II-1790-20 antisense oligonucleotides complementary to human ribonucleotide reductase R2 mRNA
gaaaccaaataaatcaagct

SEQ ID NO: 88 - DNA, 20 bp, Artificial Sequence
  AS-II-1819-20 antisense oligonucleotides complementary to human ribonucleotide reductase R2 mRNA
ttagtggtcaggagaatgta

SEQ ID NO: 89 - DNA, 20 bp, Artificial Sequence
  AS-II-1976-20 antisense oligonucleotides complementary to human ribonucleotide reductase R2 mRNA
tggcaccaactgactaatat

SEQ ID NO: 90 - DNA, 20 bp, Artificial Sequence
  AS-II-1989-20 antisense oligonucleotides complementary to human ribonucleotide reductase R2 mRNA
cctgtcttctatctggcacc

SEQ ID NO: 91 - DNA, 20 bp, Artificial Sequence
  AS-II-2009-20 antisense oligonucleotides complementary to human ribonucleotide reductase R2 mRNA
gccacaggataaaaacacaa

SEQ ID NO: 92 - DNA, 20 bp, Artificial Sequence
  AS-II-2026-20 antisense oligonucleotides complementary to human ribonucleotide reductase R2 mRNA
cccaggacactacacaagcc

SEQ ID NO: 93 - DNA, 20 bp, Artificial Sequence
  AS-II-2044-20 antisense oligonucleotides complementary to human ribonucleotide reductase R2 mRNA
tcagagggggcagagaatcc

SEQ ID NO: 94 - DNA, 20 bp, Artificial Sequence
  AS-II-2067-20 antisense oligonucleotides complementary to human ribonucleotide reductase R2 mRNA
tcctttatcccacaacactc

SEQ ID NO: 95 - DNA, 20 bp, Artificial Sequence
  AS-II-2083-20 antisense oligonucleotides complementary to human ribonucleotide reductase R2 mRNA
ccttgccctgagagattcct

SEQ ID NO: 96 - DNA, 20 bp, Artificial Sequence
  Partially Phosphorothioated AS-II-2083-20 antisenseoligonucleotides complementary to human ribonucleotide reductaseR2 mRNA
ccttgccctgagagattcct

SEQ ID NO: 97 - DNA, 20 bp, Artificial Sequence
  AS-II-2128-20 antisense oligonucleotides complementary to human ribonucleotide reductase R2 mRNA
ggcccagatcacccctaaat

SEQ ID NO: 98 - DNA, 20 bp, Artificial Sequence
  AS-II-2151-20 antisense oligonucleotides complementary to human ribonucleotide reductase R2 mRNA
aaacggcttctcacacatat

SEQ ID NO: 99 - DNA, 20 bp, Artificial Sequence
  AS-II-2164-20 antisense oligonucleotides complementary to human ribonucleotide reductase R2 mRNA
gagaaataaaatgaaacggc

SEQ ID NO: 100 - DNA, 20 bp, Artificial Sequence
  AS-II-2182-20 antisense oligonucleotides complementary to human ribonucleotide reductase R2 mRNA
cgttgaggaaaatacagtga

SEQ ID NO: 101 - DNA, 20 bp, Artificial Sequence
  AS-II-2229A-20 antisense oligonucleotides complementary to human ribonucleotide reductase R2 mRNA
gctcccacatatgaaaactc

SEQ ID NO: 102 - DNA, 20 bp, Artificial Sequence
  AS-II-2372-20 antisense oligonucleotides complementary to human ribonucleotide reductase R2 mRNA
cacacaacctacttacacca

SEQ ID NO: 103 - DNA, 20 bp, Artificial Sequence
  Antisense oligonucleotides complementary to human ribonucleotide reductase R2 mRNA
tcctggaagatcctcctcgc

SEQ ID NO: 104 - DNA, 20 bp, Artificial Sequence
  Antisense oligonucleotides complementary to human ribonucleotide reductase R2 mRNA
tcccacatatgagaaaactc

SEQ ID NO: 105 - DNA, 2500 bp, Homo sapiens
  mRNA (1)..(2500): ribonucleotide reductase R2 mRNA
cccaggcgcagccaatgggaagggtcggaggcatggcacagccaatgggaagggccggggcaccaaagccaatgggaagggccgggagcgcgcggcgcgggagatttaaaggctgctggagtgaggggtcgcccgtgcaccctgtcccagccgtcctgtcctggctgctcgctctgcttcgctgcgcctccactatgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggttgacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaaatgaactgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagattttaatgtttacttaaatataaacctggcactttacaaacaaataaacattgttttgtactcacggcggcgataatagcttgatttatttggtttctacaccaaatacattctcctgaccactaatgggagccaattcacaattcactaagtgactaaagtaagttaaacttgtgtagactaagcatgtaatttttaagttttattttaatgaattaaaatatttgttaaccaactttaaagtcagtcctgtgtatacctagatattagtcagttggtgccagatagaagacaggttgtgtttttatcctgtggcttgtgtagtgtcctgggattctctgccccctctgagtagagtgttgtgggataaaggaatctctcagggcaaggagcttcttaagttaaatcactagaaatttaggggtgatctgggccttcatatgtgtgagaagccgtttcattttatttctcactgtattttcctcaacgtctggttgatgagaaaaaattcttgaagagttttcatatgtgggagctaaggtagtattgtaaaatttcaagtcatccttaaacaaaatgatccacctaagatcttgcccctgttaagtggtgaaatcaactagaggtggttcctacaagttgttcattctagttttgtttggtgtaagtaggttgtgtgagttaattcatttatatttactatgtctgttaaatcagaaattttttattatctatgttcttctagattttacctgtagttcataaaaaaaaaaaaaaaaaaaaaaaaa